(19) United States
(12) Patent Application Publication
(10) Pub. No.: US 2023/0044917 A1
(43) Pub. Date: Feb. 9, 2023

(54) TRANSFER LEARNING-BASED USE OF PROTEIN CONTACT MAPS FOR VARIANT PATHOGENICITY PREDICTION

(71) Applicant: ILLUMINA, INC., San Diego, CA (US)

(72) Inventors: Chen CHEN, Mountain View, CA (US); Hong GAO, Palo Alto, CA (US); Laksshman S. SUNDARAM, Fremont, CA (US); Kai-How FARH, Hillsborough, CA (US)

(73) Assignee: ILLUMINA, INC., San Diego, CA (US)

(21) Appl. No.: 17/876,481

(22) Filed: Jul. 28, 2022

Related U.S. Application Data
(60) Provisional application No. 63/229,897, filed on Aug. 5, 2021.

(51) Int. Cl. G16B 40/00; G16B 30/10

(52) U.S. Cl. CPC G16B 40/00; G16B 30/10

(57) ABSTRACT

The technology disclosed relates to a variant pathogenicity prediction network. The variant pathogenicity classifier includes memory, a variant encoding sub-network, a protein contact map generation sub-network, and a pathogenicity scoring sub-network. The memory stores a reference amino acid sequence of a protein, and an alternative amino acid sequence of the protein that contains a variant amino acid caused by a variant nucleotide. The variant encoding sub-network is configured to process the alternative amino acid sequence, and generate a processed representation of the alternative amino acid sequence. The protein contact map generation sub-network is configured to process the reference amino acid sequence and the processed representation of the alternative amino acid sequence, and generate a protein contact map of the protein. The pathogenicity scoring sub-network is configured to process the protein contact map, and generate a pathogenicity indication of the variant amino acid.

### PRIORITY APPLICATION

This application claims the benefit of U.S. Provisional Patent Application No. 63/229,897, titled “TRANSFER LEARNING-BASED USE OF PROTEIN CONTACT MAPS FOR VARIANT PATHOGENICITY PREDICTION,” filed Aug. 5, 2021 (Attorney Docket No. ILLM 1042-1/IP-2074-PRV). The priority application is hereby incorporated by reference for all purposes.

### INCORPORATIONS

The following are incorporated by reference for all purposes as if fully set forth herein: U.S. patent application Ser. No. ______, titled “DEEP LEARNING-BASED USE OF PROTEIN CONTACT MAPS FOR VARIANT PATHOGENICITY PREDICTION,” filed contemporaneously (Attorney Docket No. ILLM 1049-2/IP-2155-US);

U.S. patent application Ser. No. 17/232,056, titled “DEEP CONVOLUTIONAL NEURAL NETWORKS TO PREDICT VARIANT PATHOGENICITY USING THREE-DIMENSIONAL (3D) PROTEIN STRUCTURES,” filed Apr. 15, 2021 (Attorney Docket No. ILLM 1037-2/IP-2051-US);

Sundaram, L et al. Predicting the clinical impact of human mutation with deep neural networks. Nat. Genet. 50, 1161-1170 (2018);

Jaganathan, K et al. Predicting splicing from primary sequence with deep learning. Cell 176, 535-548 (2019);

U.S. Patent Application No. 62/573,144, titled “TRAINING A DEEP PATHOGENICITY CLASSIFIER USING LARGE-SCALE BENIGN TRAINING DATA,” filed Oct. 16, 2017 (Attorney Docket No. ILLM 1000-1/IP-1611-PRV);

U.S. Patent Application No. 62/573,149, titled “PATHOGENICITY CLASSIFIER BASED ON DEEP CONVOLUTIONAL NEURAL NETWORKS (CNNs),” filed Oct. 16, 2017 (Attorney Docket No. ILLM 1000-2/IP-1612-PRV);

U.S. Patent Application No. 62/573,153, titled “DEEP SEMI-SUPERVISED LEARNING THAT GENERATES LARGE-SCALE PATHOGENIC TRAINING DATA,” filed Oct. 16, 2017 (Attorney Docket No. ILLM 1000-3/IP-1613-PRV);

U.S. Patent Application No. 62/582,898, titled “PATHOGENICITY CLASSIFICATION OF GENOMIC DATA USING DEEP CONVOLUTIONAL NEURAL NETWORKS (CNNs),” filed Nov. 7, 2017 (Attorney Docket No. ILLM 1000-4/IP-1618-PRV);

U.S. patent application Ser. No. 16/160,903, titled “DEEP LEARNING-BASED TECHNIQUES FOR TRAINING DEEP CONVOLUTIONAL NEURAL NETWORKS,” filed on Oct. 15, 2018 (Attorney Docket No. ILLM 1000-5/IP-1611-US);

U.S. patent application Ser. No. 16/160,986, titled “DEEP CONVOLUTIONAL NEURAL NETWORKS FOR VARIANT CLASSIFICATION,” filed on Oct. 15, 2018 (Attorney Docket No. ILLM 1000-6/IP-1612-US);

U.S. patent application Ser. No. 16/160,968, titled “SEMI-SUPERVISED LEARNING FOR TRAINING AN ENSEMBLE OF DEEP CONVOLUTIONAL NEURAL NETWORKS,” filed on Oct. 15, 2018 (Attorney Docket No. ILLM 1000-7/IP-1613-US); and

U.S. patent application Ser. No. 16/407,149, titled “DEEP LEARNING-BASED TECHNIQUES FOR PRE-TRAINING DEEP CONVOLUTIONAL NEURAL NETWORKS,” filed May 8, 2019 (Attorney Docket No. ILLM 1010-1/IP-1734-US).

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using deep convolutional neural networks to analyze tensorized protein data for variant pathogenicity prediction, including protein contact maps.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Genomics, in the broad sense, also referred to as functional genomics, aims to characterize the function of every genomic element of an organism by using genome-scale assays such as genome sequencing, transcriptome profiling and proteomics. Genomics arose as a data-driven science—it operates by discovering novel properties from explorations of genome-scale data rather than by testing preconceived models and hypotheses. Applications of genomics include finding associations between genotype and phenotype, discovering biomarkers for patient stratification, predicting the function of genes, and charting biochemically active genomic regions such as transcriptional enhancers.

Genomics data are too large and too complex to be mined solely by visual investigation of pairwise correlations. Instead, analytical tools are required to support the discovery of unanticipated relationships, to derive novel hypotheses and models and to make predictions. Unlike some algorithms, in which assumptions and domain expertise are hard coded, machine learning algorithms are designed to automatically detect patterns in data. Hence, machine learning algorithms are suited to data-driven sciences and, in particular, to genomics. However, the performance of machine learning algorithms can strongly depend on how the data are represented, that is, on how each variable (also called a feature) is computed. For instance, to classify a tumor as malign or benign from a fluorescent microscopy image, a preprocessing algorithm could detect cells, identify the cell type, and generate a list of cell counts for each cell type.

A machine learning model can take the estimated cell counts, which are examples of handcrafted features, as input features to classify the tumor. A central issue is that classification performance depends heavily on the quality and the relevance of these features. For example, relevant visual features such as cell morphology, distances between cells or localization within an organ are not captured in cell counts, and this incomplete representation of the data may reduce classification accuracy.

Deep learning, a subdiscipline of machine learning, addresses this issue by embedding the computation of features into the machine learning model itself to yield end-to-end models. This outcome has been realized through the development of deep neural networks, machine learning models that comprise successive elementary operations, which compute increasingly more complex features by taking the results of preceding operations as input. Deep neural networks are able to improve prediction accuracy by discovering relevant features of high complexity, such as the cell morphology and spatial organization of cells in the above example. The construction and training of deep neural networks have been enabled by the explosion of data, algorithmic advances, and substantial increases in computational capacity, particularly through the use of graphical processing units (GPUs).

The goal of supervised learning is to obtain a model that takes features as input and returns a prediction for a so-called target variable. An example of a supervised learning problem is one that predicts whether an intron is spliced out or not (the target) given features on the RNA such as the presence or absence of the canonical splice site sequence, the location of the splicing branchpoint or intron length. Training a machine learning model refers to learning its parameters, which commonly involves minimizing a loss function on training data with the aim of making accurate predictions on unseen data.

For many supervised learning problems in computational biology, the input data can be represented as a table with multiple columns, or features, each of which contains numerical or categorical data that are potentially useful for making predictions. Some input data are naturally represented as features in a table (such as temperature or time), whereas other input data need to be first transformed (such as deoxyribonucleic acid (DNA) sequence into k-mer counts) using a process called feature extraction to fit a tabular representation. For the intron-splicing prediction problem, the presence or absence of the canonical splice site sequence, the location of the splicing branchpoint and the intron length can be preprocessed features collected in a tabular format. Tabular data are standard for a wide range of supervised machine learning models, ranging from simple linear models, such as logistic regression, to more flexible nonlinear models, such as neural networks and many others.

Logistic regression is a binary classifier, that is, a supervised learning model that predicts a binary target variable. Specifically, logistic regression predicts the probability of the positive class by computing a weighted sum of the input features mapped to the [0,1] interval using the sigmoid function, a type of activation function. The parameters of logistic regression, or other linear classifiers that use different activation functions, are the weights in the weighted sum. Linear classifiers fail when the classes, for instance, that of an intron spliced out or not, cannot be well discriminated with a weighted sum of input features. To improve predictive performance, new input features can be manually added by transforming or combining existing features in new ways, for example, by taking powers or pairwise products.

Neural networks use hidden layers to learn these nonlinear feature transformations automatically. Each hidden layer can be thought of as multiple linear models with their output transformed by a nonlinear activation function, such as the sigmoid function or the more popular rectified-linear unit (ReLU). Together, these layers compose the input features into relevant complex patterns, which facilitates the task of distinguishing two classes.

Deep neural networks use many hidden layers, and a layer is said to be fully-connected when each neuron receives inputs from all neurons of the preceding layer. Neural networks are commonly trained using stochastic gradient descent, an algorithm suited to training models on very large data sets. Implementation of neural networks using modern deep learning frameworks enables rapid prototyping with different architectures and data sets. Fully-connected neural networks can be used for a number of genomics applications, which include predicting the percentage of exons spliced in for a given sequence from sequence features such as the presence of binding motifs of splice factors or sequence conservation; prioritizing potential disease-causing genetic variants; and predicting cis-regulatory elements in a given genomic region using features such as chromatin marks, gene expression and evolutionary conservation.

Local dependencies in spatial and longitudinal data must be considered for effective predictions. For example, shuffling a DNA sequence or the pixels of an image severely disrupts informative patterns. These local dependencies set spatial or longitudinal data apart from tabular data, for which the ordering of the features is arbitrary. Consider the problem of classifying genomic regions as bound versus unbound by a particular transcription factor, in which bound regions are defined as high-confidence binding events in chromatin immunoprecipitation following by sequencing (ChIP-seq) data. Transcription factors bind to DNA by recognizing sequence motifs. A fully-connected layer based on sequence-derived features, such as the number of k-mer instances or the position weight matrix (PWM) matches in the sequence, can be used for this task. As k-mer or PWM instance frequencies are robust to shifting motifs within the sequence, such models could generalize well to sequences with the same motifs located at different positions. However, they would fail to recognize patterns in which transcription factor binding depends on a combination of multiple motifs with well-defined spacing. Furthermore, the number of possible k-mers increases exponentially with k-mer length, which poses both storage and overfitting challenges.

A convolutional layer is a special form of fully-connected layer in which the same fully-connected layer is applied locally, for example, in a 6 bp window, to all sequence positions. This approach can also be viewed as scanning the sequence using multiple PWMs, for example, for transcription factors GATA1 and TAL1. By using the same model parameters across positions, the total number of parameters is drastically reduced, and the network is able to detect a motif at positions not seen during training. Each convolutional layer scans the sequence with several filters by producing a scalar value at every position, which quantifies the match between the filter and the sequence. As in fully-connected neural networks, a nonlinear activation function (commonly ReLU) is applied at each layer. Next, a pooling operation is applied, which aggregates the activations in contiguous bins across the positional axis, commonly taking the maximal or average activation for each channel. Pooling reduces the effective sequence length and coarsens the signal. The subsequent convolutional layer composes the output of the previous layer and is able to detect whether a GATA1 motif and TAL1 motif were present at some distance range. Finally, the output of the convolutional layers can be used as input to a fully-connected neural network to perform the final prediction task. Hence, different types of neural network layers (e.g., fully-connected layers and convolutional layers) can be combined within a single neural network.

Convolutional neural networks (CNNs) can predict various molecular phenotypes on the basis of DNA sequence alone. Applications include classifying transcription factor binding sites and predicting molecular phenotypes such as chromatin features, DNA contact maps, DNA methylation, gene expression, translation efficiency, RBP binding, and microRNA (miRNA) targets. In addition to predicting molecular phenotypes from the sequence, convolutional neural networks can be applied to more technical tasks traditionally addressed by handcrafted bioinformatics pipelines. For example, convolutional neural networks can predict the specificity of guide RNA, denoise ChIP-seq, enhance Hi-C data resolution, predict the laboratory of origin from DNA sequences and call genetic variants. Convolutional neural networks have also been employed to model long-range dependencies in the genome. Although interacting regulatory elements may be distantly located on the unfolded linear DNA sequence, these elements are often proximal in the actual 3D chromatin conformation. Hence, modelling molecular phenotypes from the linear DNA sequence, albeit a crude approximation of the chromatin, can be improved by allowing for long-range dependencies and allowing the model to implicitly learn aspects of the 3D organization, such as promoter-enhancer looping. This is achieved by using dilated convolutions, which have a receptive field of up to 32 kb. Dilated convolutions also allow splice sites to be predicted from sequence using a receptive field of 10 kb, thereby enabling the integration of genetic sequence across distances as long as typical human introns (See Jaganathan, K. et al. Predicting splicing from primary sequence with deep learning. Cell 176, 535-548 (2019)).

Different types of neural network can be characterized by their parameter-sharing schemes. For example, fully-connected layers have no parameter sharing, whereas convolutional layers impose translational invariance by applying the same filters at every position of their input. Recurrent neural networks (RNNs) are an alternative to convolutional neural networks for processing sequential data, such as DNA sequences or time series, that implement a different parameter-sharing scheme. Recurrent neural networks apply the same operation to each sequence element. The operation takes as input the memory of the previous sequence element and the new input. It updates the memory and optionally emits an output, which is either passed on to subsequent layers or is directly used as model predictions. By applying the same model at each sequence element, recurrent neural networks are invariant to the position index in the processed sequence. For example, a recurrent neural network can detect an open reading frame in a DNA sequence regardless of the position in the sequence. This task requires the recognition of a certain series of inputs, such as the start codon followed by an in-frame stop codon.

The main advantage of recurrent neural networks over convolutional neural networks is that they are, in theory, able to carry over information through infinitely long sequences via memory. Furthermore, recurrent neural networks can naturally process sequences of widely varying length, such as mRNA sequences. However, convolutional neural networks combined with various tricks (such as dilated convolutions) can reach comparable or even better performances than recurrent neural networks on sequence-modelling tasks, such as audio synthesis and machine translation. Recurrent neural networks can aggregate the outputs of convolutional neural networks for predicting single-cell DNA methylation states, RBP binding, transcription factor binding, and DNA accessibility. Moreover, because recurrent neural networks apply a sequential operation, they cannot be easily parallelized and are hence much slower to compute than convolutional neural networks.

Each human has a unique genetic code, though a large portion of the human genetic code is common for all humans In some cases, a human genetic code may include an outlier, called a genetic variant, that may be common among individuals of a relatively small group of the human population. For example, a particular human protein may comprise a specific sequence of amino acids, whereas a variant of that protein may differ by one amino acid in the otherwise same specific sequence.

Genetic variants may be pathogenetic, leading to diseases. Though most of such genetic variants have been depleted from genomes by natural selection, an ability to identify which genetic variants are likely to be pathogenic can help researchers focus on these genetic variants to gain an understanding of the corresponding diseases and their diagnostics, treatments, or cures. The clinical interpretation of millions of human genetic variants remains unclear. Some of the most frequent pathogenic variants are single nucleotide missense mutations that change the amino acid of a protein. However, not all missense mutations are pathogenic.

Models that can predict molecular phenotypes directly from biological sequences can be used as in silico perturbation tools to probe the associations between genetic variation and phenotypic variation and have emerged as new methods for quantitative trait loci identification and variant prioritization. These approaches are of major importance given that the majority of variants identified by genome-wide association studies of complex phenotypes are non-coding, which makes it challenging to estimate their effects and contribution to phenotypes. Moreover, linkage disequilibrium results in blocks of variants being co-inherited, which creates difficulties in pinpointing individual causal variants. Thus, sequence-based deep learning models that can be used as interrogation tools for assessing the impact of such variants offer a promising approach to find potential drivers of complex phenotypes. One example includes predicting the effect of non-coding single-nucleotide variants and short insertions or deletions (indels) indirectly from the difference between two variants in terms of transcription factor binding, chromatin accessibility or gene expression predictions. Another example includes predicting novel splice site creation from sequence or quantitative effects of genetic variants on splicing.

End-to-end deep learning approaches for variant effect predictions are applied to predict the pathogenicity of missense variants from protein sequence and sequence conservation data (See Sundaram, L et al. Predicting the clinical impact of human mutation with deep neural networks. Nat. Genet. 50, 1161-1170 (2018), referred to herein as “PrimateAI”). PrimateAI uses deep neural networks trained on variants of known pathogenicity with data augmentation using cross-species information. In particular, PrimateAI uses sequences of wild-type and mutant proteins to compare the difference and decide the pathogenicity of mutations using the trained deep neural networks. Such an approach which utilizes the protein sequences for pathogenicity prediction is promising because it can avoid the circularity problem and overfitting to previous knowledge. However, compared to the adequate number of data to train the deep neural networks effectively, the number of clinical data available in ClinVar is relatively small. To overcome this data scarcity, PrimateAI uses common human variants and variants from primates as benign data while simulated variants based on trinucleotide context were used as unlabeled data.

PrimateAI outperforms prior methods when trained directly upon sequence alignments. PrimateAI learns important protein domains, conserved amino acid positions, and sequence dependencies directly from the training data consisting of about 120,000 human samples. PrimateAI substantially exceeds the performance of other variant pathogenicity prediction tools in differentiating benign and pathogenic de-novo mutations in candidate developmental disorder genes, and in reproducing prior knowledge in ClinVar. These results suggest that PrimateAI is an important step forward for variant classification tools that may lessen the reliance of clinical reporting on prior knowledge.

Central to protein biology is the understanding of how structural elements give rise to observed function. The surfeit of protein structural data enables development of computational methods to systematically derive rules governing structural-functional relationships. However, performance of these methods depends critically on the choice of protein structural representation.

Protein sites are microenvironments within a protein structure, distinguished by their structural or functional role. A site can be defined by a location and a local neighborhood around this location in which the structure or function exists. Central to rational protein engineering is the understanding of how the structural arrangement of amino acids creates functional characteristics within protein sites. Determination of the structural and functional roles of individual amino acids within a protein provides information to help engineer and alter protein functions. Identifying functionally or structurally important amino acids allows focused engineering efforts such as site-directed mutagenesis for altering targeted protein functional properties. Alternatively, this knowledge can help avoid engineering designs that would abolish a desired function.

Since it has been established that structure is far more conserved than sequence, the increase in protein structural data provides an opportunity to systematically study the underlying pattern governing the structural-functional relationships using data-driven approaches. A fundamental aspect of any computational protein analysis is how protein structural information is represented. The performance of machine learning methods often depends more on the choice of data representation than the machine learning algorithm employed. Good representations efficiently capture the most critical information while poor representations create a noisy distribution with no underlying patterns.

Proteins in 3D space can be considered complex systems that emerged through the interactions of their constituent amino acids. This representation provides a powerful framework to uncover the general organized principle of protein contact network. Protein residue-residue contact prediction is the problem of predicting whether any two residues in a protein sequence are spatially close to each other in the folded 3D protein structure. By analyzing whether or not a residue pair in a protein sequence is in contact (i.e., close in 3D space), we are able to form protein contact maps.

The surfeit of protein structures and the recent success of deep learning algorithms provide an opportunity to develop tools for automatically extracting task specific representations of protein structures. Therefore, an opportunity arises to predict variant pathogenicity using tensorized protein data, including protein contact maps, as input to deep neural networks.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The color drawings also may be available in PAIR via the Supplemental Content tab. In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which.

FIG. 1A depicts one implementation of training a protein contact map generation sub-network on the task of protein contact map generation to produce a so-called “trained” protein contact map generation sub-network.

FIG. 1B illustrates one implementation of using transfer learning to further train the trained protein contact map generation sub-network on the task of variant pathogenicity prediction to produce a so-called “cross-trained” protein contact map generation sub-network for use in training a variant pathogenicity prediction network.

FIG. 1C shows one implementation of applying the trained variant pathogenicity prediction network at inference.

FIG. 1D shows two globular proteins with some contacts in them shown in black dotted lines along with the contact distance in Angstrom (Å).

FIG. 2A depicts an example architecture of the protein contact map generation sub-network, in accordance with one implementation of the technology disclosed.

FIG. 2B illustrates an example residual block, in accordance with one implementation of the technology disclosed.

FIG. 3 depicts an example architecture of the variant pathogenicity prediction network, in accordance with one implementation of the technology disclosed.

FIG. 4 shows an example of reference amino acid sequence of a protein and an example of an alternative amino acid sequence of the protein, in accordance with one implementation of the technology disclosed.

FIG. 5 illustrates respective one-hot encodings of a reference amino acid sequence and an alternative amino acid sequence processed as input by the variant pathogenicity prediction network, in accordance with one implementation of the technology disclosed.

FIG. 6 depicts an example 3-state secondary structure profile processed as input by the variant pathogenicity prediction network, in accordance with one implementation of the technology disclosed.

FIG. 7 shows an example 3-state solvent accessibility profile processed as input by the variant pathogenicity prediction network, in accordance with one implementation of the technology disclosed.

FIG. 8 illustrates an example position-specific frequency matrix (PSFM) processed as input by the variant pathogenicity prediction network, in accordance with one implementation of the technology disclosed.

FIG. 9 depicts an example position-specific scoring matrix (PSSM) processed as input by the variant pathogenicity prediction network, in accordance with one implementation of the technology disclosed.

FIG. 10 shows one implementation of generating the PSFM and the PSSM.

FIG. 11 illustrates an example PSFM encoding processed as input by the variant pathogenicity prediction network, in accordance with one implementation of the technology disclosed.

FIG. 12 depicts an example PSSM encoding processed as input by the variant pathogenicity prediction network, in accordance with one implementation of the technology disclosed.

FIG. 13 shows an example CCMpred encoding processed as input by the variant pathogenicity prediction network, in accordance with one implementation of the technology disclosed.

FIG. 14 illustrates an example of tensorized protein data processed as input by the variant pathogenicity prediction network, in accordance with one implementation of the technology disclosed.

FIG. 15 depicts an example ground truth protein contact map used to train the protein contact map generation sub-network, in accordance with one implementation of the technology disclosed.

FIG. 16 shows an example predicted protein contact map generated by the protein contact map generation sub-network, in accordance with one implementation of the technology disclosed.

FIG. 17 is one implementation of the so-called “outer concatenation” operation used by the protein contact map generation sub-network for converting sequential features to pairwise features.

FIGS. 18(a)-(d) represent the steps in constructing the protein contact maps.

FIGS. 19(a)-(d) represent the relationship between a 2D protein contact map (FIG. 19(b)) and the corresponding 3D protein structure (FIG. 19(a)).

FIGS. 20, 21, 22, 23, 24, 25, and 26 illustrate different examples of 2D protein contact maps representing corresponding 3D protein structures.

FIG. 27 graphically elucidates the notion that pathogenic variants, though distributed in a spatially distance manner along a linear/sequential amino acid sequence, tend to cluster in certain regions of the 3D protein structure, making protein contact maps contributive to the task of variant pathogenicity prediction.

FIG. 28 depicts a pathogenicity classifier that makes variant pathogenicity classifications at least in part based on protein contact maps generated by the trained protein contact map generation sub-network.

FIG. 29 depicts an example network architecture of the pathogenicity classifier, in accordance with one implementation of the technology disclosed.

FIG. 30 is a flowchart that executes one implementation of a computer-implemented method of variant pathogenicity prediction.

FIG. 31 is a flowchart that executes one implementation of a computer-implemented method of variant pathogenicity classification.

FIG. 32 shows performance results achieved by different implementations of the variant pathogenicity prediction network on the task of variant pathogenicity prediction, as applied on different test data sets.

FIG. 33 shows performance results achieved by different implementations of the pathogenicity classifier on the task of variant pathogenicity classification, as applied on different test sets.

FIG. 34 is an example computer system that can be used to implement the technology disclosed.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The detailed description of various implementations will be better understood when read in conjunction with the appended drawings. To the extent that the figures illustrate diagrams of the functional blocks of the various implementations, the functional blocks are not necessarily indicative of the division between hardware circuitry. Thus, for example, one or more of the functional blocks (e.g., modules, processors, or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various implementations are not limited to the arrangements and instrumentality shown in the drawings.

The processing engines and databases of the figures, designated as modules, can be implemented in hardware or software, and need not be divided up in precisely the same blocks as shown in the figures. Some of the modules can also be implemented on different processors, computers, or servers, or spread among a number of different processors, computers, or servers. In addition, it will be appreciated that some of the modules can be combined, operated in parallel or in a different sequence than that shown in the figures without affecting the functions achieved. The modules in the figures can also be thought of as flowchart steps in a method. A module also need not necessarily have all its code disposed contiguously in memory; some parts of the code can be separated from other parts of the code with code from other modules or other functions disposed in between.

This section is organized as follows. We first provide a brief overview of some implementations of the technology disclosed. We then provide a detailed discussion of protein contact maps. This is followed by some transfer learning implementations and details of some example architectures of different sub-networks that work in tandem to make variant pathogenicity predictions. This is followed by example encodings of different inputs like PSSMs, PSFMs, CCMPred, and so on that are processed as inputs by the different sub-networks. What follows is a discussion of how 2D protein contact maps are proxies of 3D protein structures and therefore contribute to solving the problem of variant pathogenicity determination. Finally, we disclose a pathogenicity classifier that is trained without the disclosed transfer learning implementation and processes protein contact maps generated by another network. Some test results are also disclosed as indicia of inventiveness and non-obviousness.

### Introduction

Two-dimensional (2D) protein contact maps are proxies of three-dimensional (3D) protein structures because they capture 3D spatial proximity of those residue pairs that are sequentially distant in protein sequences, along with capturing other forms of short-range, medium-range, and long-range contacts. In some proteins, certain pathogenic amino acid variants that are sequentially distant in the amino acid sequences have been observed to spatially cluster in the corresponding 3D protein structures. Accordingly, we propose that 2D protein contact maps contribute to variant pathogenicity prediction. Specifically, we present deep neural networks that are trained to generate variant pathogenicity predictions as outputs in response to processing 2D protein contact maps as inputs. In one implementation, our variant pathogenicity prediction network is configured with one-dimensional (1D) residual blocks that generate residue-wise features, and with 2D residual blocks that generate residue pair-wise features. We also generate a so-called “cross-trained” protein contact map generator using transfer learning. This cross-trained protein contact map generator is first trained on the task of protein contact map generation, and then on the task of variant pathogenicity prediction.

### Protein Contact Map Prediction

Proteins are represented by a collection of atoms and their coordinates in three-dimensional (3D) space. An amino acid can have a variety of atoms, such as carbon atoms, oxygen (O) atoms, nitrogen (N) atoms, and hydrogen (H) atoms. The atoms can be further classified as side chain atoms and backbone atoms. The backbone carbon atoms can include alpha-carbon (C_{α}) atoms and beta-carbon (C_{β}) atoms.

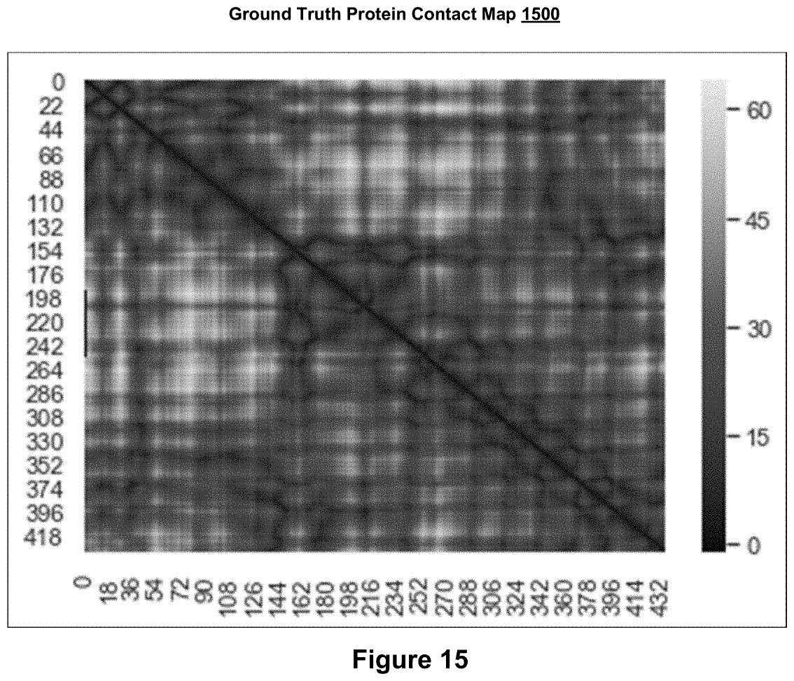

A “protein contact map” (or simply “contact map”) represents the distance between all possible amino acid residue pairs of a 3D protein structure using a binary two-dimensional matrix. For two residues i and j, the ij^{th} element of the matrix is 1 if the two residues are closer than a predetermined threshold, and 0 otherwise. Various contact definitions have been proposed—the distance between the Cα-Cα atom with threshold 6-12 Å; distance between Cβ-Cβ atoms with threshold 6-12 Å (Cα is used for Glycine); and distance between the side-chain centers of mass. FIGS. 15, 16, 18, 19, 20, 21, 22, 23, and 24 show different examples of protein contact maps.

Protein contact maps provide a more reduced representation of a protein structure than its full 3D atomic coordinates. The advantage is that protein contact maps are invariant to rotations and translations, which makes them more easily predictable by machine learning methods. It has also been shown that under certain circumstances (e.g., low content of erroneously predicted contacts) it is possible to reconstruct the 3D coordinates of a protein using its protein contact map. Protein contact maps are also used for protein superimposition and to describe similarity between protein structures. They are either predicted from protein sequence or calculated from a given structure.

A protein contact map describes the pairwise spatial and functional relationship of amino acids (residues) in a protein and contains key information for protein 3D structure prediction. Two residues of a protein are in contact if their Euclidean distance is <8 Å, in some implementations. The distance of two residues can be calculated using Cα or Cβ atoms, corresponding to Cα- or Cβ-based contacts. A protein contact map can also be considered a binary L×L matrix, where L is the protein length. In this matrix, an element with value 1 indicates the corresponding two residues are in contact; otherwise, they are not in contact.

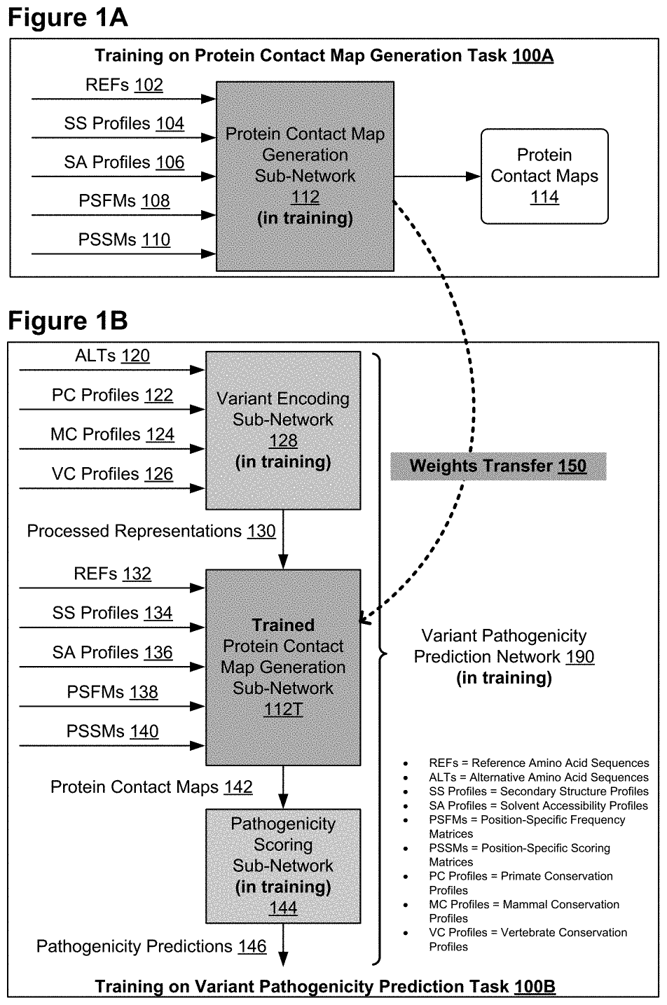

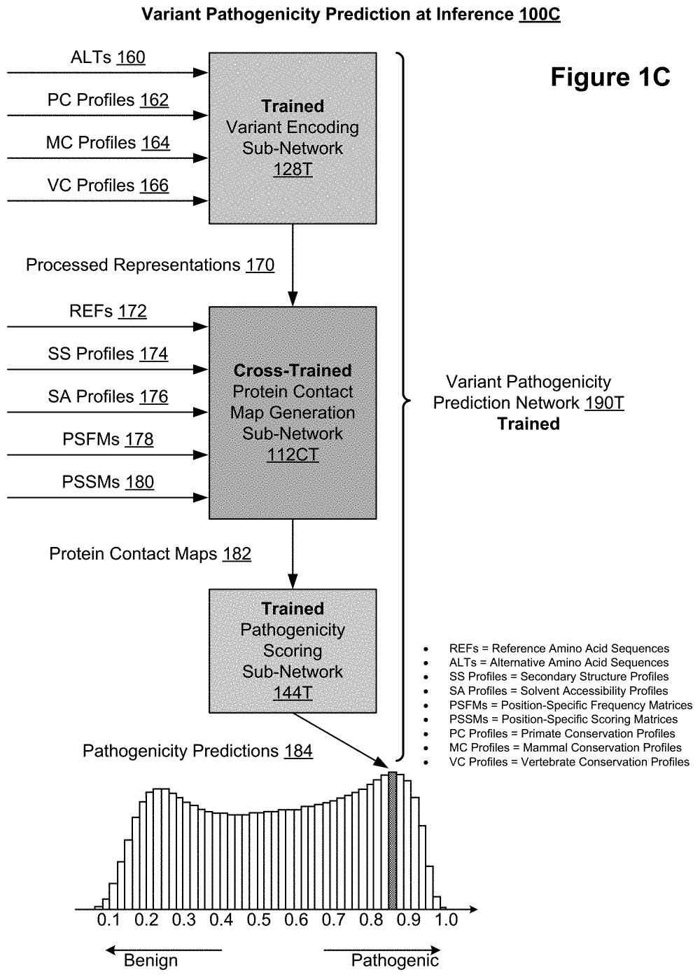

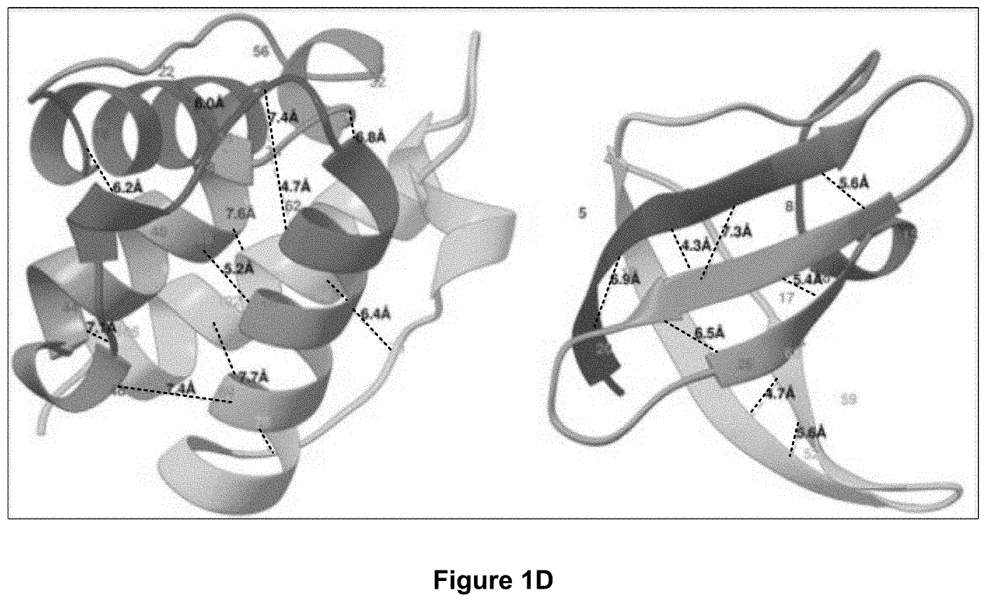

A 3D structure of a protein is expressed as x, y, and z coordinates of the amino acids' atoms, and hence, contacts can be defined using a distance threshold. FIG. 1D shows two globular proteins with some contacts in them shown in black dotted lines along with the contact distance in Angstrom (Å). The alpha helical protein 1bkr (left) has many long-range contacts and the beta sheet protein 1c9o (right) has more short- and medium-range contacts. Contacts occurring between sequentially distant residues, i.e., the long-range contacts, impose strong constraints on the 3D structure of a protein and are particularly important for structural analyses, understanding the folding process, and predicting the 3D structure.

In some implementations, a minimum sequence separation in the corresponding protein sequence can also be defined so that sequentially close residues, which are spatially close as well, are excluded. Although proteins can be better reconstructed with Cβ atoms, Cα atoms, being backbone atoms, are widely used. The choice of distance threshold and sequence separation threshold also defines the number of contacts in a protein. At lower distance thresholds, a protein has fewer number of contacts and at a smaller sequence separation threshold, the protein has many local contacts. In the Critical Assessment of Techniques for Protein Structure Prediction (CASP) competition, a pair of residues are defined as a contact if the distance between their Cβ atoms is less than or equal to 8 Å, provided they are separated by at least five residues in the sequence. In other instances, a pair of residues are said to be in contact if their Cα atoms are separated by at least 7 Å with no minimum sequence separation distance defined.

Realizing that the contacting residues which are far apart in the protein sequence but close together in the 3D space are important for protein folding, contacts are widely categorized as short-range, medium-range, and long-range. Short-range contacts are those separated by 6-11 residues in the sequence; medium-range contacts are those separated by 12-23 residues, and long-range contacts are those separated by at least 24 residues. Long-range contacts are often evaluated separately as they are the most important of the three and also the hardest to predict. Depending upon the 3D shape (fold), some proteins have a lot of short-range contacts while others have more long-range contacts, as shown in FIG. 1D.

Besides the three categories of contacts, the total number of contacts in a protein is also important for reconstructing 3D models for the protein. Certain proteins, such as those having long tail-like structures, have fewer contacts and are difficult to reconstruct even using true contacts while others, for example compact globular proteins, have a lot of contacts, and can be reconstructed with high accuracy. Another important element of predicted contacts is the coverage of contacts, i.e., how well the contacts are distributed over the structure of a protein. A set of contacts having low coverage will have most of the contacts clustered in a specific region of the structure, which means that even if all predicted contacts are correct, we may still need additional information to reconstruct the protein with high accuracy.

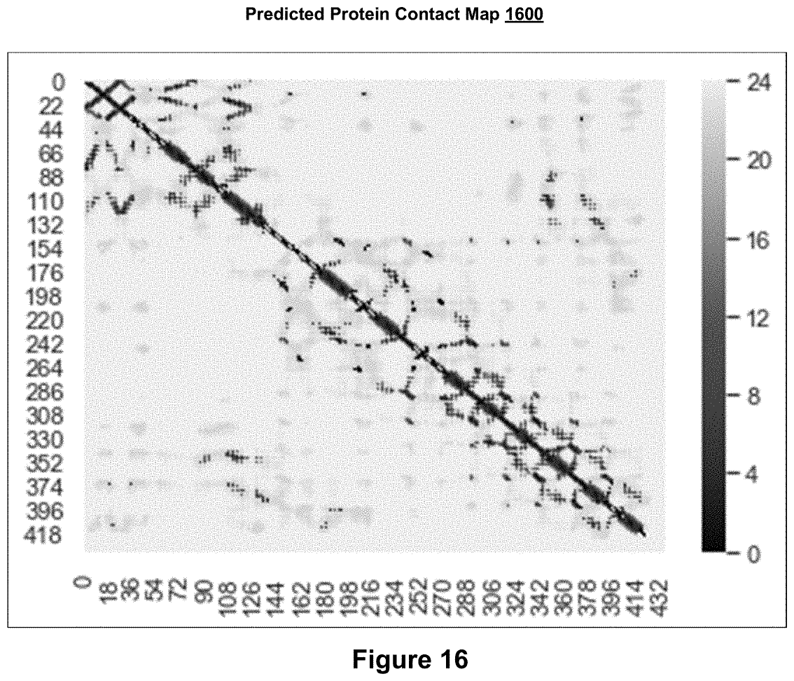

FIG. 1A depicts one implementation of training a protein contact map generation sub-network 112 on the task of protein contact map generation 100A to produce a so-called “trained” protein contact map generation sub-network 112T. In one implementation, the protein contact map generation sub-network 112 is trained to process, as input, at least one of: (i) reference amino acid sequences (REFs) 102 of proteins, (ii) secondary structure (SS) profiles 104 of the proteins, (iii) solvent accessibility (SA) profiles 106 of the proteins, (iv) position-specific frequency matrices (PSFMs) 108 of the proteins, and (v) position-specific scoring matrices (PSSMs) 110 of the proteins, and generate, as output, protein contact maps 114. FIG. 16 shows an example predicted protein contact map 1600 generated by the protein contact map generation sub-network, in accordance with one implementation of the technology disclosed. Position-specific scoring matrices (PSSMs) are sometimes also referred to as position-specific weight matrices (PSWMs) or position weight matrices (PWMs).

In one implementation, the protein contact map generation sub-network 112 is trained on reference amino acid sequences of bacteria proteins (e.g., 30000 bacteria proteins) with known protein contact maps that can be used as ground truth during the training. FIG. 15 depicts an example ground truth protein contact map 1500 used to train the protein contact map generation sub-network 112, in accordance with one implementation of the technology disclosed.

In some implementations, the protein contact map generation sub-network 112 is trained using a mean squared error loss function that minimizes error between known protein contact maps and protein contact maps predicted by the protein contact map generation sub-network 112 during the training. In other implementations, the protein contact map generation sub-network 112 is trained using a mean absolute error loss function that minimizes error between the known protein contact maps and protein contact maps predicted by the protein contact map generation sub-network during the training.

In one implementation, the protein contact map generation sub-network 112 is a neural network. In another implementation, the protein contact map generation sub-network 112 uses convolutional neural networks (CNNs) with a plurality of convolution layers. In another implementation, the protein contact map generation sub-network 112 uses recurrent neural networks (RNNs) such as a long short-term memory networks (LSTMs), bi-directional LSTMs (Bi-LSTMs), and gated recurrent units (GRU)s. In yet another implementation, the protein contact map generation sub-network 112 uses both the CNNs and the RNNs. In yet another implementation, the protein contact map generation sub-network 112 uses graph-convolutional neural networks that model dependencies in graph-structured data. In yet another implementation, the protein contact map generation sub-network 112 uses variational autoencoders (VAEs). In yet another implementation, the protein contact map generation sub-network 112 uses generative adversarial networks (GANs). In yet another implementation, the protein contact map generation sub-network 112 can also be a language model based, for example, on self-attention such as the one implemented by Transformers and BERTs. In yet another implementation, the protein contact map generation sub-network 112 uses a fully connected neural network (FCNN).

In yet other implementations, the protein contact map generation sub-network 112 can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The protein contact map generation sub-network 112 can use one or more loss functions such as logistic regression/log loss, multi-class cross-entropy/softmax loss, binary cross-entropy loss, L1 loss, L2 loss, smooth L1 loss, and Huber loss. It can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The protein contact map generation sub-network 112 can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, attention mechanisms, and gaussian error linear unit.

The protein contact map generation sub-network 112 can be trained using backpropagation-based gradient update techniques, in some implementations. Example gradient descent techniques that can be used for training the protein contact map generation sub-network 112 include stochastic gradient descent (SGD), batch gradient descent, and mini-batch gradient descent. Some examples of gradient descent optimization algorithms that can be used to train the protein contact map generation sub-network 112 are Momentum, Nesterov accelerated gradient, Adagrad, Adadelta, RMSprop, Adam, AdaMax, Nadam, and AMSGrad. In other implementations, the protein contact map generation sub-network 112 can be trained by unsupervised learning, semi-supervised learning, self-learning, reinforcement learning, multitask learning, multimodal learning, transfer learning, knowledge distillation, and so on.

### Transfer Learning

The process of reusing or transferring weights learnt from one task into another task is called transfer learning. Transfer learning thus refers to extracting the learnt weights from a trained base network (pretrained model) and transferring them to another untrained target network instead of training the target network from scratch. Transfer learning can be used either by (a) using the pretrained model as a fixed feature extractor, or by (b) fine-tuning the whole model. In the former scenario, for example, the last fully connected layer (the classifier layer) of the pretrained model is replaced with a new classifier layer that is then trained on a new dataset In this way, the feature extraction layers of the pretrained model remain fixed and only the new classifier layer gets fine-tuned. In the latter scenario, the whole network, i.e., the feature extraction layers of the pretrained model and the new classifier layer, are retrained on the new dataset by continuing backpropagation up to the feature extraction layers of the pretrained model. In this way, all weights of the whole network are fine-tubed for the new task.

The technology disclosed first trains the protein contact map generation sub-network 112 on the task of protein contact map generation 100A (FIG. 1A), and then retrains the trained protein contact map generation sub-network 112T on the task of variant pathogenicity prediction 100B (FIG. 1B). The retraining includes incorporating the trained protein contact map generation sub-network 112T into a larger variant pathogenicity prediction network 190 that includes additional subnetworks (e.g., a variant encoding sub-network 128, a pathogenicity scoring sub-network 144), and joint training the sub-networks 128, 112T, and 144 end-to-end on the task of variant pathogenicity prediction 100B to produce a so-called “trained” variant pathogenicity prediction network 190T.

This way, FIG. 1A can be considered a “pre-training” stage of the protein contact map generation sub-network 112 in which weights (coefficients) of the protein contact map generation sub-network 112 are learnt on the task of protein contact map generation 100A, and FIG. 1B can be considered a “transfer learning” stage of the trained protein contact map generation sub-network 112T in which learnt weights of the trained protein contact map generation sub-network 112T are further trained (or transferred) 150 on the task of variant pathogenicity prediction 100B.

A person skilled in the art will appreciate that the sub-networks 128, 112T, and 144 can be arranged in any order in the variant pathogenicity prediction network 190. A person skilled in the art will also appreciate that the variant pathogenicity prediction network 190 can include additional layers or sub-networks.

The following discussion focuses on one implementation of training the variant pathogenicity prediction network 190 in which—(i) the variant encoding sub-network 128 is trained to process a first input, and generate a processed presentation of the first input, (ii) the trained protein contact map generation sub-network 112T is further trained to process a second input and the processed presentation of the first input, and generate a protein contact map, and (iii) the pathogenicity scoring sub-network 144 is trained to process the protein contact map, and generate a pathogenicity prediction.

In one implementation, the first input processed by the variant encoding sub-network 128 can include at least one of: (i) alternative amino acid sequences 120 of proteins in training data that contain variant amino acids caused by variant nucleotides, (ii) amino acid-wise primate conservation profiles 122 of the proteins, (iii) amino acid-wise mammal conservation profiles 124 of the proteins, and (iv) amino acid-wise vertebrate conservation profiles 126 of the proteins. The resulting output produced by the variant encoding sub-network 128 in response to processing the first input are processed representations 130 of the first input. The processed representations 130 can be convolved features (or activations), in some implementations.

In one implementation, the second input processed by the trained protein contact map generation sub-network 112T can include at least one of: (i) reference amino acid sequences (REFs) 132 of the proteins, (ii) secondary structure (SS) profiles 134 of the proteins, (iii) solvent accessibility (SA) profiles 136 of the proteins, (iv) position-specific frequency matrices (PSFMs) 138 of the proteins, and (v) position-specific scoring matrices (PSSMs) 140 of the proteins. The resulting output produced by the trained protein contact map generation sub-network 112T in response to processing the second input and the processed representations 130 of the first input are protein contact maps 142.

In one implementation, the pathogenicity scoring sub-network 144 is trained to process the protein contact maps 142, and generate pathogenicity predictions 146 as output. The pathogenicity predictions 146 indicate a degree of pathogenicity (or benignness) of the variant amino acids in the training data.

FIG. 1C shows one implementation of applying the trained variant pathogenicity prediction network 190T at inference 100C. The following discussion focuses on one implementation of the trained variant pathogenicity prediction network 190T in which—(i) the trained variant encoding sub-network 128T is configured to process a first input, and generate a processed presentation of the first input, (ii) the “cross-trained” protein contact map generation sub-network 112CT is configured to process a second input and the processed presentation of the first input, and generate a protein contact map, and (iii) the trained pathogenicity scoring sub-network 144T is configured to process the protein contact map, and generate a pathogenicity prediction. The term “cross-trained” refers to the notion that the protein contact map generation sub-network 112 is trained on both: (a) the task of protein contact map generation 100A, and (b) the task of variant pathogenicity prediction 100B.

In one implementation, the first input processed by the trained variant encoding sub-network 128T can include at least one of: (i) alternative amino acid sequences 160 of proteins in inference data (e.g., unknown protein contact maps of human proteins) that contain variant amino acids caused by variant nucleotides, (ii) amino acid-wise primate conservation profiles 162 of the proteins (e.g., PSFMs determined from alignment to only homologous primate sequences), (iii) amino acid-wise mammal conservation profiles 164 of the proteins (e.g., PSFMs determined from alignment to only homologous mammal sequences), and (iv) amino acid-wise vertebrate conservation profiles 166 of the proteins (e.g., PSFMs determined from alignment to only homologous vertebrate sequences). The resulting output produced by the trained variant encoding sub-network 128T in response to processing the first input are processed representations 170 of the first input. The processed representations 170 can be convolved features (or activations), in some implementations.

In one implementation, the second input processed by the cross-trained protein contact map generation sub-network 112CT can include at least one of: (i) reference amino acid sequences (REFs) 172 of the proteins, (ii) secondary structure (SS) profiles 174 of the proteins, (iii) solvent accessibility (SA) profiles 176 of the proteins, (iv) position-specific frequency matrices (PSFMs) 178 of the proteins, and (v) position-specific scoring matrices (PSSMs) 180 of the proteins. The resulting output produced by the cross-trained protein contact map generation sub-network 112CT in response to processing the second input and the processed representations 170 of the first input are protein contact maps 182.

In one implementation, the trained pathogenicity scoring sub-network 144T is configured to process the protein contact maps 182, and generate pathogenicity predictions 184 as output. The pathogenicity predictions 184 indicate a degree of pathogenicity (or benignness) of the variant amino acids in the inference data.

In one implementation, the variant encoding sub-network 128 is a neural network. In another implementation, the variant encoding sub-network 128 uses convolutional neural networks (CNNs) with a plurality of convolution layers. In another implementation, the variant encoding sub-network 128 uses recurrent neural networks (RNNs) such as a long short-term memory networks (LSTMs), bi-directional LSTMs (Bi-LSTMs), and gated recurrent units (GRU)s. In yet another implementation, the variant encoding sub-network 128 uses both the CNNs and the RNNs. In yet another implementation, the variant encoding sub-network 128 uses graph-convolutional neural networks that model dependencies in graph-structured data. In yet another implementation, the variant encoding sub-network 128 uses variational autoencoders (VAEs). In yet another implementation, the variant encoding sub-network 128 uses generative adversarial networks (GANs). In yet another implementation, the variant encoding sub-network 128 can also be a language model based, for example, on self-attention such as the one implemented by Transformers and BERTs. In yet another implementation, the variant encoding sub-network 128 uses a fully connected neural network (FCNN).

In yet other implementations, the variant encoding sub-network 128 can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The variant encoding sub-network 128 can use one or more loss functions such as logistic regression/log loss, multi-class cross-entropy/softmax loss, binary cross-entropy loss, L1 loss, L2 loss, smooth L1 loss, and Huber loss. It can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The variant encoding sub-network 128 can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, attention mechanisms, and gaussian error linear unit.

The variant encoding sub-network 128 can be trained using backpropagation-based gradient update techniques, in some implementations. Example gradient descent techniques that can be used for training the variant encoding sub-network 128 include stochastic gradient descent (SGD), batch gradient descent, and mini-batch gradient descent. Some examples of gradient descent optimization algorithms that can be used to train the variant encoding sub-network 128 are Momentum, Nesterov accelerated gradient, Adagrad, Adadelta, RMSprop, Adam, AdaMax, Nadam, and AMSGrad. In other implementations, the variant encoding sub-network 128 can be trained by unsupervised learning, semi-supervised learning, self-learning, reinforcement learning, multitask learning, multimodal learning, transfer learning, knowledge distillation, and so on.

In one implementation, the pathogenicity scoring sub-network 144 is a neural network. In another implementation, the pathogenicity scoring sub-network 144 uses convolutional neural networks (CNNs) with a plurality of convolution layers. In another implementation, the pathogenicity scoring sub-network 144 uses recurrent neural networks (RNNs) such as a long short-term memory networks (LSTMs), bi-directional LSTMs (Bi-LSTMs), and gated recurrent units (GRU)s. In yet another implementation, the pathogenicity scoring sub-network 144 uses both the CNNs and the RNNs. In yet another implementation, the pathogenicity scoring sub-network 144 uses graph-convolutional neural networks that model dependencies in graph-structured data. In yet another implementation, the pathogenicity scoring sub-network 144 uses variational autoencoders (VAEs). In yet another implementation, the pathogenicity scoring sub-network 144 uses generative adversarial networks (GANs). In yet another implementation, the pathogenicity scoring sub-network 144 can also be a language model based, for example, on self-attention such as the one implemented by Transformers and BERTs. In yet another implementation, the pathogenicity scoring sub-network 144 uses a fully connected neural network (FCNN).

In yet other implementations, the pathogenicity scoring sub-network 144 can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The pathogenicity scoring sub-network 144 can use one or more loss functions such as logistic regression/log loss, multi-class cross-entropy/softmax loss, binary cross-entropy loss, L1 loss, L2 loss, smooth L1 loss, and Huber loss. It can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The pathogenicity scoring sub-network 144 can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, attention mechanisms, and gaussian error linear unit.

The pathogenicity scoring sub-network 144 can be trained using backpropagation-based gradient update techniques, in some implementations. Example gradient descent techniques that can be used for training the pathogenicity scoring sub-network 144 include stochastic gradient descent (SGD), batch gradient descent, and mini-batch gradient descent. Some examples of gradient descent optimization algorithms that can be used to train the pathogenicity scoring sub-network 144 are Momentum, Nesterov accelerated gradient, Adagrad, Adadelta, RMSprop, Adam, AdaMax, Nadam, and AMSGrad. In other implementations, the pathogenicity scoring sub-network 144 can be trained by unsupervised learning, semi-supervised learning, self-learning, reinforcement learning, multitask learning, multimodal learning, transfer learning, knowledge distillation, and so on.

In one implementation, the variant pathogenicity prediction network 190 is a neural network. In another implementation, the variant pathogenicity prediction network 190 uses convolutional neural networks (CNNs) with a plurality of convolution layers. In another implementation, the variant pathogenicity prediction network 190 uses recurrent neural networks (RNNs) such as a long short-term memory networks (LSTMs), bi-directional LSTMs (Bi-LSTMs), and gated recurrent units (GRU)s. In yet another implementation, the variant pathogenicity prediction network 190 uses both the CNNs and the RNNs. In yet another implementation, the variant pathogenicity prediction network 190 uses graph-convolutional neural networks that model dependencies in graph-structured data. In yet another implementation, the variant pathogenicity prediction network 190 uses variational autoencoders (VAEs). In yet another implementation, the variant pathogenicity prediction network 190 uses generative adversarial networks (GANs). In yet another implementation, the variant pathogenicity prediction network 190 can also be a language model based, for example, on self-attention such as the one implemented by Transformers and BERTs. In yet another implementation, the variant pathogenicity prediction network 190 uses a fully connected neural network (FCNN).

In yet other implementations, the variant pathogenicity prediction network 190 can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The variant pathogenicity prediction network 190 can use one or more loss functions such as logistic regression/log loss, multi-class cross-entropy/softmax loss, binary cross-entropy loss, L1 loss, L2 loss, smooth L1 loss, and Huber loss. It can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The variant pathogenicity prediction network 190 can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, attention mechanisms, and gaussian error linear unit.

The variant pathogenicity prediction network 190 can be trained using backpropagation-based gradient update techniques, in some implementations. Example gradient descent techniques that can be used for training the variant pathogenicity prediction network 190 include stochastic gradient descent (SGD), batch gradient descent, and mini-batch gradient descent. Some examples of gradient descent optimization algorithms that can be used to train the variant pathogenicity prediction network 190 are Momentum, Nesterov accelerated gradient, Adagrad, Adadelta, RMSprop, Adam, AdaMax, Nadam, and AMSGrad. In other implementations, the variant pathogenicity prediction network 190 can be trained by unsupervised learning, semi-supervised learning, self-learning, reinforcement learning, multitask learning, multimodal learning, transfer learning, knowledge distillation, and so on.

### Example Architecture of Protein Contact Map Generation Sub-Network

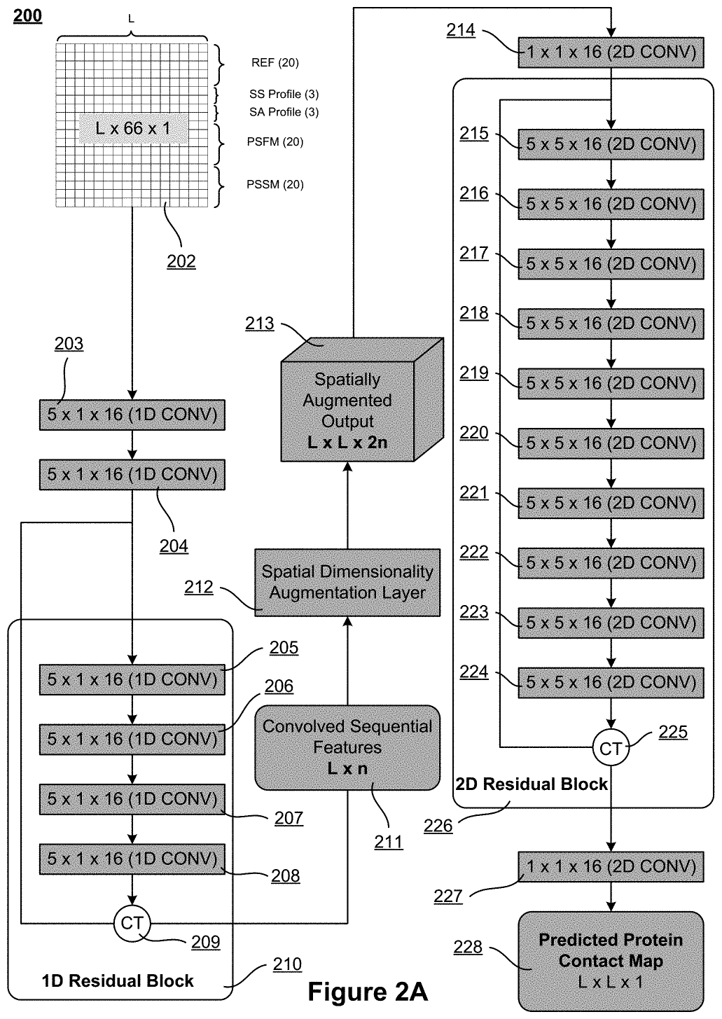

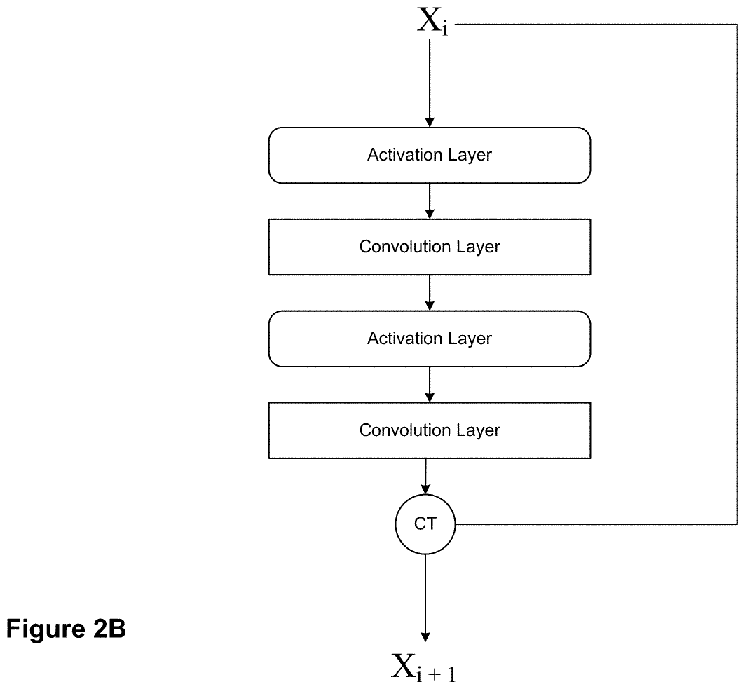

FIG. 2A depicts an example architecture 200 of the protein contact map generation sub-network 112, in accordance with one implementation of the technology disclosed. In one implementation, an input 202 to the protein contact map generation sub-network 112 comprises a reference amino acid sequence of a protein-under-analysis, a 3-state secondary structure profile of the protein-under-analysis, a 3-state solvent accessibility profile of the protein-under-analysis, a position-specific frequency matrix (PSFM) of the protein-under-analysis, and a position-specific scoring matrix (PSSM) of the protein-under-analysis. In one implementation, the input 202 is a tensor that concatenates: (i) a L×20×1 matrix of a one-hot encoding of the reference amino acid sequence (where L is the number of amino acids in the reference amino acid sequence and 20 denotes the twenty amino acid categories), (ii) L×3×1 matrix of a 3-state encoding of the 3-state secondary structure profile (where the 3 states are helix, beta sheet, and coil), (iii) L×3×1 matrix of a 3-state encoding of the 3-state solvent accessibility profile (where the 3 states are buried, intermediate, and exposed), (iv) L×20×1 matrix of the PSFM, and (v) L×20×1 matrix of the PSFM. The resulting concatenated tensor 202 is of size L×66×1, in accordance with some implementations.

The tensor 202 is processed by one or more initial 1D convolution layers (e.g., 1D convolution layers 203 and 204). In the illustrated example, each of the 1D convolution layers 203 and 204 has 16 convolution filters that each operate on a window of size 5×1.

The output of the second 1D convolution layer 204 is fed as input to a 1D residual block 210. The 1D residual block 210 conducts a series of 1D convolutions (e.g., four 1D convolutions 205, 206, 207, and 208) of sequential features in the output of the second 1D convolution layer 204, along with intermediate concatenations (CTs) 209. As used herein, concatenation operations can include combining by concatenation (stitching), summing, or multiplication.

FIG. 2B shows an example of a residual block comprising two convolution layers and two activation layers. In FIG. 2B, Xₗ and Xₗ₊₁ are the input and output of the residual block, respectively. An activation layer conducts a nonlinear transformation of its input without using any parameters. One example of the nonlinear transformation is rectified linear (ReLU) activation function. Let f(Xₗ) denote the result of Xₗ going through the two activation layers and the two convolution layers. Then, Xₗ₊₁ is equal to Xₗ+f(Xₗ). That is, Xₗ₊₁ is a combination of Xₗ and its nonlinear transformation. Since f(Xₗ) is equal to the difference between Xₗ₊₁ and Xₗ, f is called a residual function and this logic is called a residual block (or a residual network or a residual sub-network).

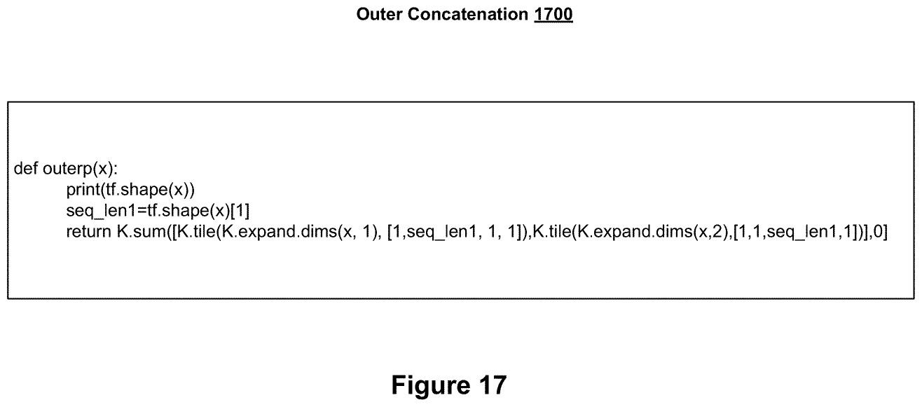

The output of the 1D residual block 210 is illustrated herein as so-called “convolved sequential features” 211, which have a dimensionality of L×n. The convolved sequential features 211 are converted to 2D matrix by a so-called “outer concatenation”—an operation similar to outer product. The outer concatenation is implemented by a spatial dimensionality augmentation layer 212. The outer concatenation converts sequential features to pairwise features. Let v={v₁, v₂, . . . , vᵢ, . . . , v_{L}} be the final output of the 1D residual network, i.e., the convolved sequential features 211, where L is the protein sequence length and vᵢ is a feature vector storing the output information for amino acid i. For a pair of amino acids i and j, the outer concatenation concatenates vᵢ, v_{(i+j)/2} and vⱼ to a single vector and use it as one input feature of this amino acid pair. FIG. 17 is one implementation of the outer concatenation 1700 operation used by the protein contact map generation sub-network 112 for converting sequential features to pairwise features. In some implementations, the input features for this amino acid pair also include mutual information, for example, the evolutionary coupling (EC) information calculated, for example, by CCMpred and pair-wise contact potential

The output of the spatial dimensionality augmentation layer 212 is illustrated herein as so-called “spatially augmented output” 213, which has a dimensionality of L×L×2n, with twice as many spatial dimensions as the convolved sequential features 211 with a dimensionality of L×n.

The spatially augmented output 213 is fed as input to a 2D residual block 226, in some implementations, after processing by one or more initial 2D convolution layers (e.g., 2D convolution layer 214). The 2D residual block 226 conducts a series of 2D convolutions (e.g., ten 1D convolutions 215, 216, 217, 218, 219, 220, 221, 222, 223, and 224) of the spatially augmented output 213, along with intermediate concatenations (CTs) 225. As used herein, concatenation operations can include combining by concatenation (stitching), summing, or multiplication. In the illustrated example, each of the 2D convolution layers 215-224 has 16 convolution filters that each operate on a window of size 5×5.

The output of the 2D residual block 226 is fed as input to one or more terminal 2D convolution layers (e.g., 2D convolution layer 227), which produces a predicted protein contact map 228 as output. The predicted protein contact map 228 has a dimensionality of L×L×1.

In some implementations, each convolutional layer in the 1D and 2D residual blocks 210 and 226 is preceded by a nonlinear transformation like ReLU. Mathematically, the output of the 1D residual block 210 is a 2D matrix with dimensions L×n, where n is the number of new features (or hidden neurons/filters) generated by the last 1D convolutional layer of the 1D residual block 210. Biologically, the 1D residual block 210 learns the sequential context of an amino acid. By stacking multiple 1D convolution layers, the 1D residual block 210 learns information in a very large sequential context.

In the 2D residual block 226, the output of a 2D convolution layer has dimensions L×L×n, where n is the number of new features (or hidden neurons/filters) generated by the 2D convolution layer for one amino acid pair. The 2D residual block 226 learns contact occurrence patterns with high-order correlation (e.g., 2D context of an amino acid pair).

In the 1D residual block 210, Xₗ and Xₗ₊₁ represent sequential features and have dimensions L×nₗ and L×nₗ₊₁, respectively, where L is the protein sequence length and nₗ(nₗ₊₁) can be interpreted as the number of features or hidden neurons at each position (i.e., amino acid).

In the 2D residual block 226, Xₗ and Xₗ₊₁ represent pairwise features and have dimensions L×L×nₗ and L×L×nₗ₊₁, respectively, where nₗ(nₗ₊₁) can be interpreted as the number of features or hidden neurons at each position (i.e., amino acid pair). In some implementations, the condition nₗ≤(nₗ₊₁) is enforced, since one position at a higher level is supposed to carry more information. When nₗ<(nₗ₊₁), in calculating Xₗ+f(Xₗ), Xₗ is padded with zeros so that it has the same dimensions as Xₗ₊₁. In some implementations, to speed up training, a batch normalization layer is added before each activation layer, which normalizes the input to an activation layer to have zero mean and one standard deviation.

The number of hidden neurons/filters can vary at each convolution layer, both in the 1D and 2D residual blocks 210 and 226. In some implementations, each of the 1D and 2D residual blocks 210 and 226 can in turn comprise one or more residual blocks concatenated together.

The 1D and 2D convolution operations are matrix-vector multiplications. Let X and Y (with dimensions L×m and L×n, respectively) be the input and output of a 1D convolution layer, respectively. Let the window size be 2w+1 and s=(2w+1)m. The convolution operator that transforms X to Y can be represented as a 2D matrix with dimensions n×s, denoted as C. C is protein length-independent and each convolution layer can have a different C. Let Xᵢ be a submatrix of X centered at amino acid i (1≤i≤L) with dimensions (2w+1)×m, and Yᵢ be the i-th row of Y. The Yᵢ can be calculated by first flattening Xᵢ to a vector of length s and then multiplying C and the flattened Xᵢ.

### Example Architecture of Variant Pathogenicity Prediction Network

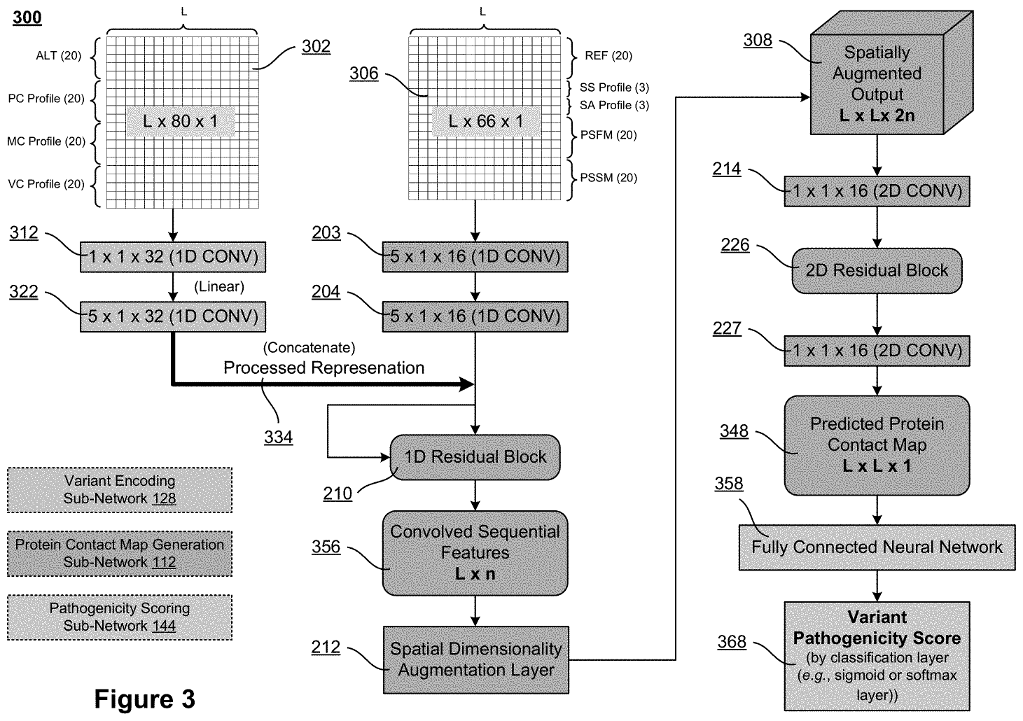

FIG. 3 depicts an example architecture 300 of the variant pathogenicity prediction network 190, in accordance with one implementation of the technology disclosed. In the illustrated example, 1D convolutions 312 and 322 form the variant encoding sub-network 128. Also, in the illustrated example, fully connected neural network 358 forms the pathogenicity scoring sub-network 144. Also, in the illustrated example, the 1D convolution layers 203 and 204, the 1D residual block 210, the spatial dimensionality augmentation layer 212, the 2D convolution layers 214 and 227, and the 2D residual block 226 form the protein contact map generation sub-network 112.

In FIG. 3, input 306 to the protein contact map generation sub-network 112 is tensorized in a similar fashion as the input 202, as discussed above.

In FIG. 3, input 302 to the variant encoding sub-network 128 comprises an alternative amino acid sequence of a protein-under-analysis that contains a variant amino acid caused by a variant nucleotide, an amino acid-wise primate conservation profile of the protein-under-analysis, an amino acid-wise mammal conservation profile of the protein-under-analysis, and an amino acid-wise vertebrate conservation profile of the protein-under-analysis. In one implementation, the input 302 is a tensor that concatenates: (i) a L×20×1 matrix of a one-hot encoding of the alternative amino acid sequence (where L is the number of amino acids in the reference amino acid sequence and 20 denotes the twenty amino acid categories), (ii) L×20×1 matrix of a PSFM determined from alignment to only homologous primate sequences, (iii) L×20×1 matrix of a PSFM determined from alignment to only homologous mammal sequences, and (iv) L×20×1 matrix of a PSFM determined from alignment to only homologous vertebrate sequences. The resulting concatenated tensor 302 is of size L×80×1, in accordance with some implementations.

The tensor 302 is processed by one or more 1D convolution layers (e.g., 1D convolutions 312 and 322) of the variant encoding sub-network 128. In the illustrated example, each of the 1D convolution layers 312 and 322 has 32 convolution filters that each operate on a window of size 5×1.

The output of the second 1D convolution layer 322 is illustrated herein as a so-called “processed representation” 334, which is fed as input to the 1D residual block 210 of the protein contact map generation sub-network 112. In some implementations, the output of the second 1D convolution layer 204 of the of the protein contact map generation sub-network 112 is concatenated with the processed representation 334, and the resulting concatenated output is fed as input to the 1D residual block 210. As used herein, concatenation operations can include combining by concatenation (stitching), summing, or multiplication.

As discussed above, the 1D residual block 210 generates convolved sequential features 356. Also, as discussed above, the spatial dimensionality augmentation layer 212 generates a spatially augmented output 308. The spatially augmented output 308 is processed through the initial 2D convolution layer 214, followed by the 2D residual block 226, and followed by the terminal 2D convolution layer 227 to generate a predicted protein contact map 348.

The predicted protein contact map 348 is processed through the fully connected neural network 358 (and a classification layer (e.g., softmax layer, sigmoid layer, or hyperbolic tangent (tanh) layer) (not shown)) of the pathogenicity scoring sub-network 144 to generate a variant pathogenicity score 368.

### One-Hot Encodings

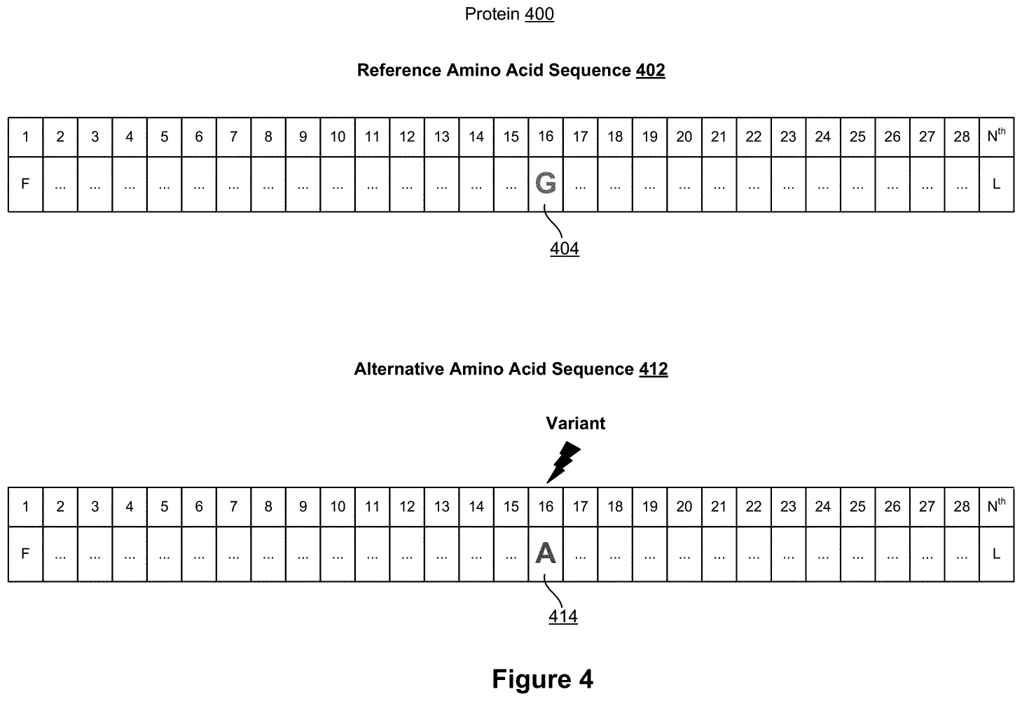

FIG. 4 shows an example of a reference amino acid sequence 402 of a protein 400 and an example of an alternative amino acid sequence 412 of the protein 400, in accordance with one implementation of the technology disclosed. The protein 400 comprises N amino acids. Positions of the amino acids in the protein 400 are labelled 1, 2, 3 . . . N. In the illustrated example, position 16 is the location that experiences an amino acid variant 414 (mutation) caused by an underlying nucleotide variant. For example, for the reference amino acid sequence 402, position 1 has reference amino acid Phenylalanine (F), position 16 has reference amino acid Glycine (G) 404, and position N (e.g., the last amino acid of the reference amino acid sequence 402) has reference amino acid Leucine (L). Though not illustrated for clarity, remaining positions in the reference amino acid sequence 402 contain various amino acids in an order that is specific to the protein 400. The alternative amino acid sequence 412 is the same as the reference amino acid sequence 402 except for the variant amino acid 414 at position 16, which contains the alternative amino acid Alanine (A) 414 instead of the reference amino acid Glycine (G) 404.

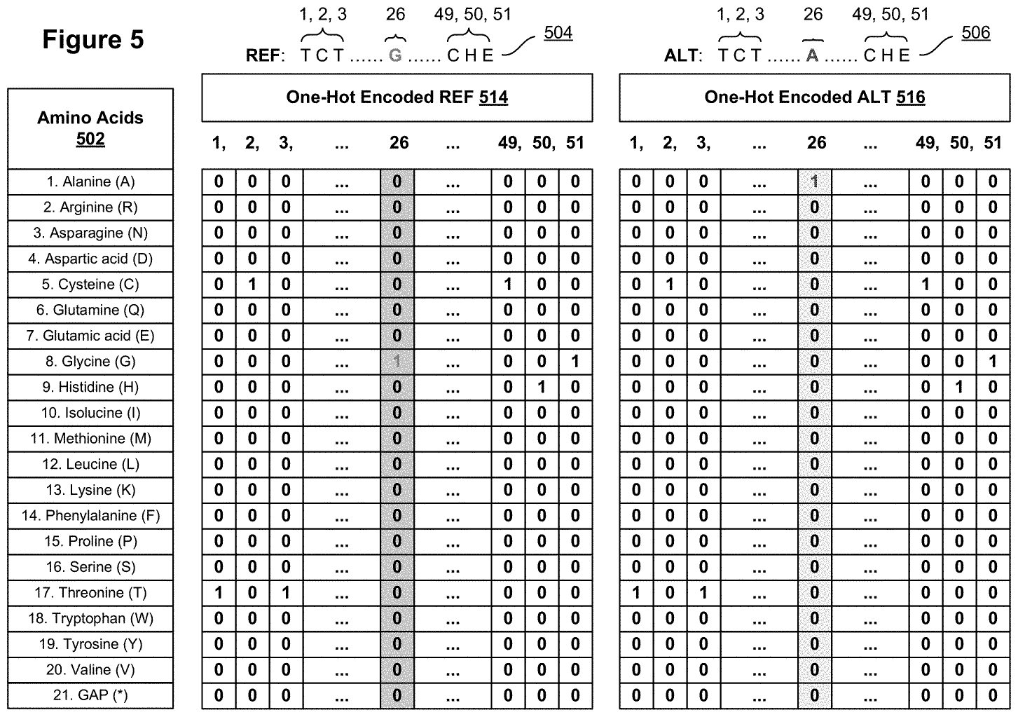

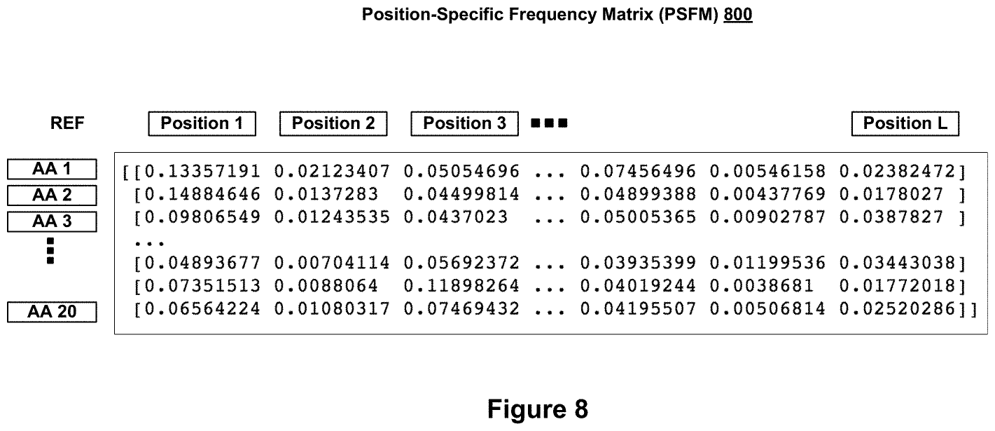

FIG. 5 illustrates respective one-hot encodings 514 and 516 of a reference amino acid sequence 504 and an alternative amino acid sequence 506 processed as input by the variant pathogenicity prediction network 190, in accordance with one implementation of the technology disclosed. In FIG. 8, the left-most column 502 lists the twenty amino acid categories corresponding to the twenty naturally occurring amino acids appearing in the genetic code, along with a twenty-first gap amino acid marker for undetermined amino acids.

In one-hot encoding, each amino acid in an amino acid sequence of size L (e.g., L=51 in FIG. 5) is encoded with a binary vector of twenty bits (or twenty-one bits including the gap amino acid), with one of the bits being hot (i.e., 1) while others being 0. The hot bit indicates that a given amino acid position in the L-length amino acid sequence belongs to a corresponding amino acid category in the twenty amino acid categories. Also note that the one-hot encoding REF 514 and the one-hot encoding ALT 516 differ only in the 26^{th} vector corresponding to the 26^{th} positions in the reference amino acid sequence 504 and the alternative amino acid sequence 506 that experience an amino acid variant, i.e., Glycine (G)→Alanine (A).

### Secondary Structure Profiles

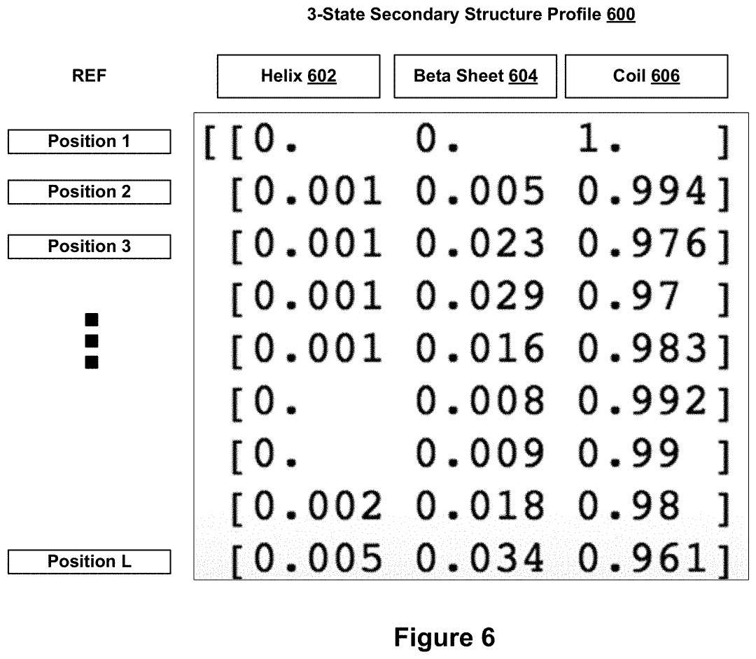

Protein secondary structure (SS) refers to the local conformation of the polypeptide backbone of proteins. There are two regular SS states: alpha helix (H) and beta sheet (B), and one irregular SS state: coils (C). FIG. 6 depicts an example 3-state secondary structure profile 600 processed as input by the variant pathogenicity prediction network 190, in accordance with one implementation of the technology disclosed. In the illustrated example, each amino acid position in an L-length reference amino acid sequence of a protein is assigned three probabilities respectively corresponding the three SS states H, B, and C. The three probabilities for each amino acid position sum to one, in some implementations.

### Solvent Accessibility Profiles

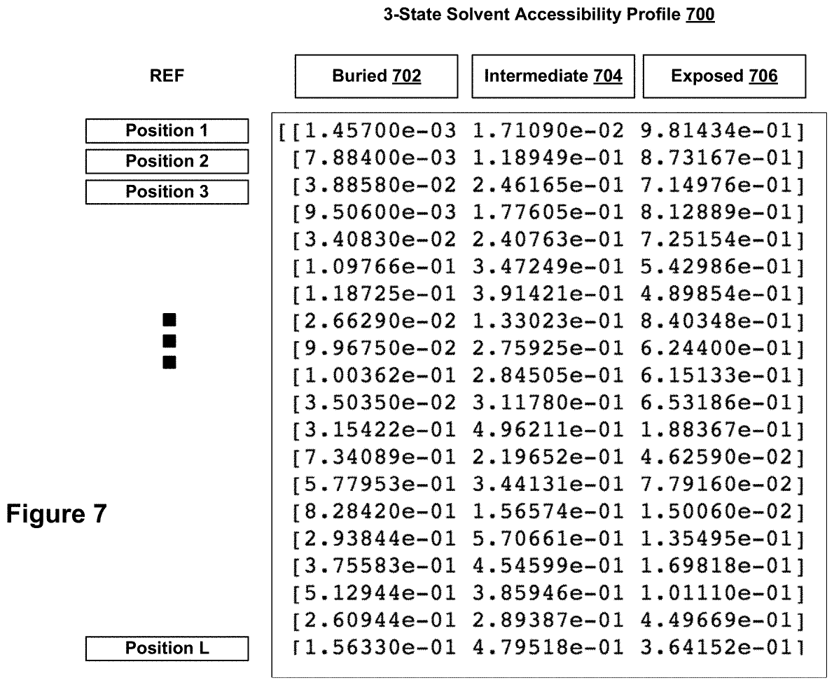

The solvent accessibility (SA) is defined as the surface region of a residue (amino acid) that is accessible to a rounded solvent while probing the surface of that residue. There are three SA states: buried (B), intermediate (I), and exposed (E). FIG. 7 shows an example 3-state solvent accessibility profile 700 processed as input by the variant pathogenicity prediction network 190, in accordance with one implementation of the technology disclosed. In the illustrated example, each amino acid position in an L-length reference amino acid sequence of a protein is assigned three probabilities respectively corresponding the three SA states B, I, and E. The three probabilities for each amino acid position sum to one, in some implementations.

### PSFMs and PSSMs

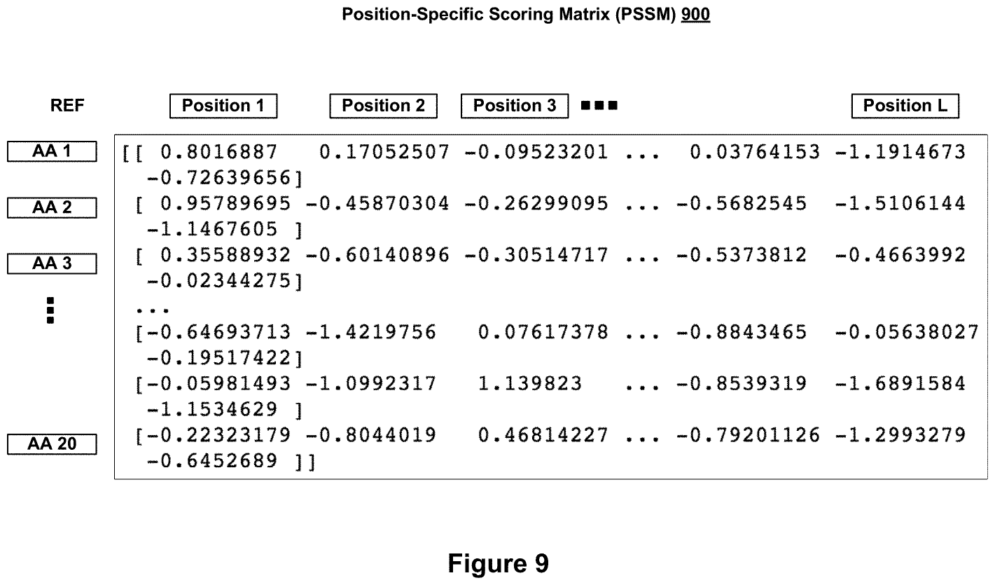

FIG. 8 illustrates an example position-specific frequency matrix (PSFM) 800 processed as input by the variant pathogenicity prediction network 190, in accordance with one implementation of the technology disclosed. FIG. 9 depicts an example position-specific scoring matrix (PSSM) 900 processed as input by the variant pathogenicity prediction network 190, in accordance with one implementation of the technology disclosed.

Multiple sequence alignment (MSA) is a sequence alignment of multiple homologous protein sequences to a target protein. MSA is an important step in comparative analyses and property prediction of biological sequences since a lot of information, for example, evolution and coevolution clusters, are generated from the MSA and can be mapped to the target sequence of choice or on the protein structure.

Sequence profiles of a protein sequence X of length L are a L×20 matrix, either in the form of a PSSM or a PSFM. The columns of a PSSM and a PSFM are indexed by the alphabet of amino acids and each row corresponds to a position in the protein sequence. PSSMs and PSFMs contain the substitution scores and the frequencies, respectively, of the amino acids at different positions in the protein sequence. Each row of a PSFM is normalized to sum to 1. The sequence profiles of the protein sequence X are computed by aligning X with multiple sequences in a protein database that have statistically significant sequence similarities with X. Therefore, the sequence profiles contain more general evolutionary and structural information of the protein family that protein sequence X belongs to, and thus, provide valuable information for remote homology detection and fold recognition.

A protein sequence (called query sequence, e.g., a reference amino acid sequence of a protein) can be used as a seed to search and align homogenous sequences from a protein database (e.g., SWISSPROT) using, for example, a PSI-BLAST program. The aligned sequences share some homogenous segments and belong to the same protein family. The aligned sequences are further converted into two profiles to express their homogeneous information: PSSM and PSFM. Both PSSM and PSFM are matrices with 20 rows and L columns, where L is the total number of amino acids in the query sequence. Each column of a PSSM represents the log-likelihood of the residue substitutions at the corresponding positions in the query sequence. The (i, j)-th entry of the PSSM matrix represents the chance of the amino acid in the j-th position of the query sequence being mutated to amino acid type i during the evolution process. A PSFM contains the weighted observation frequencies of each position of the aligned sequences. Specifically, the (i, j)-th entry of the PSFM matrix represents the possibility of having amino acid type i in position j of the query sequence.

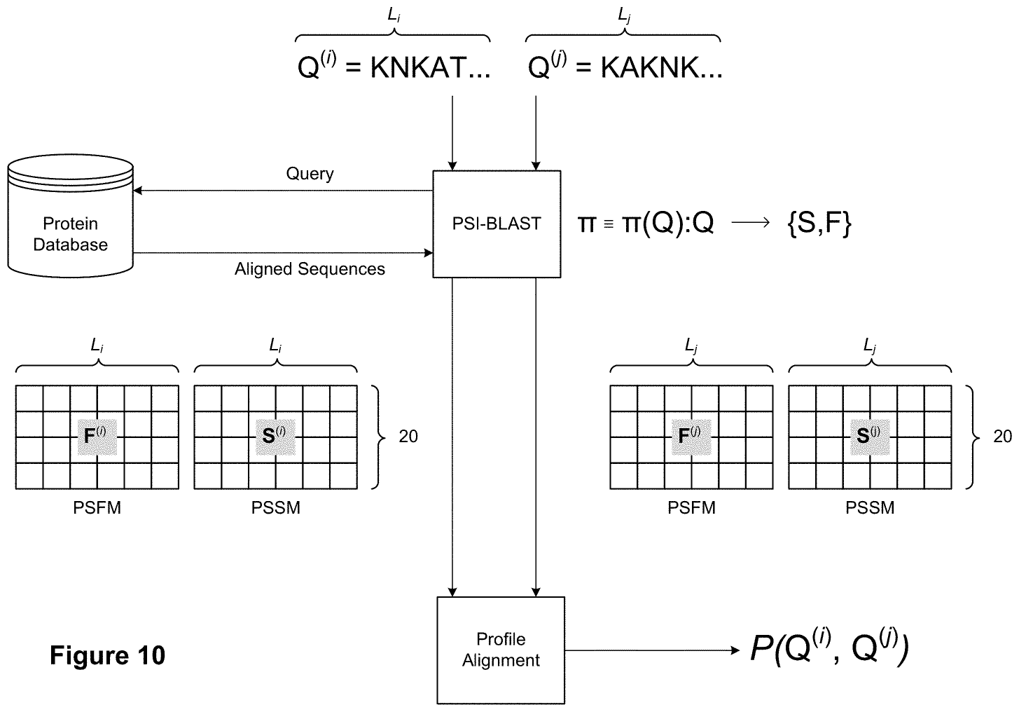

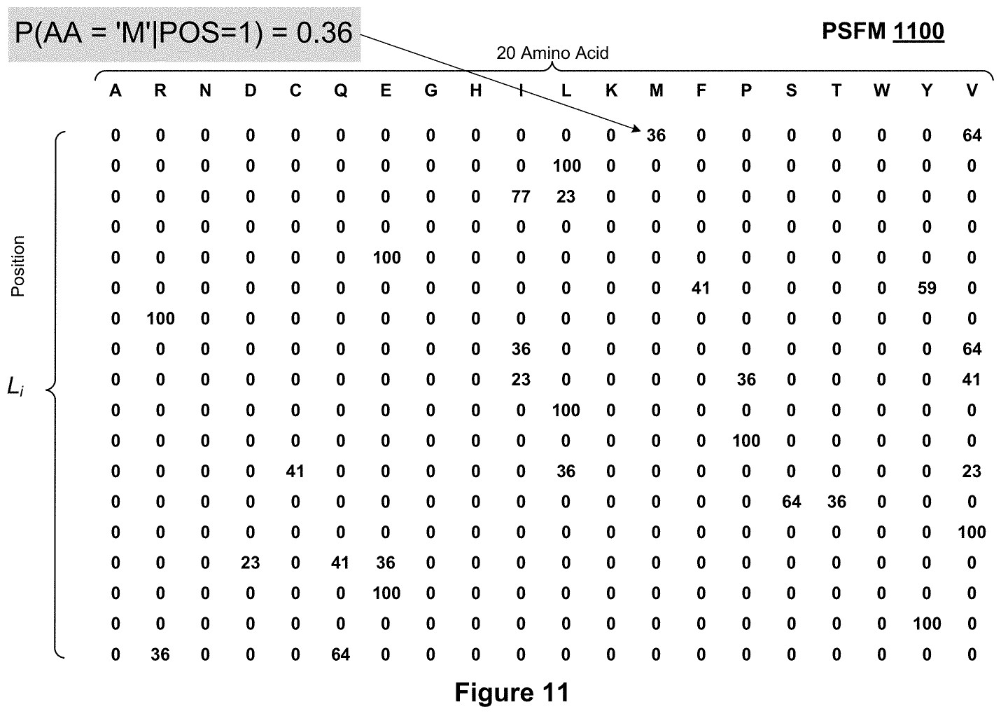

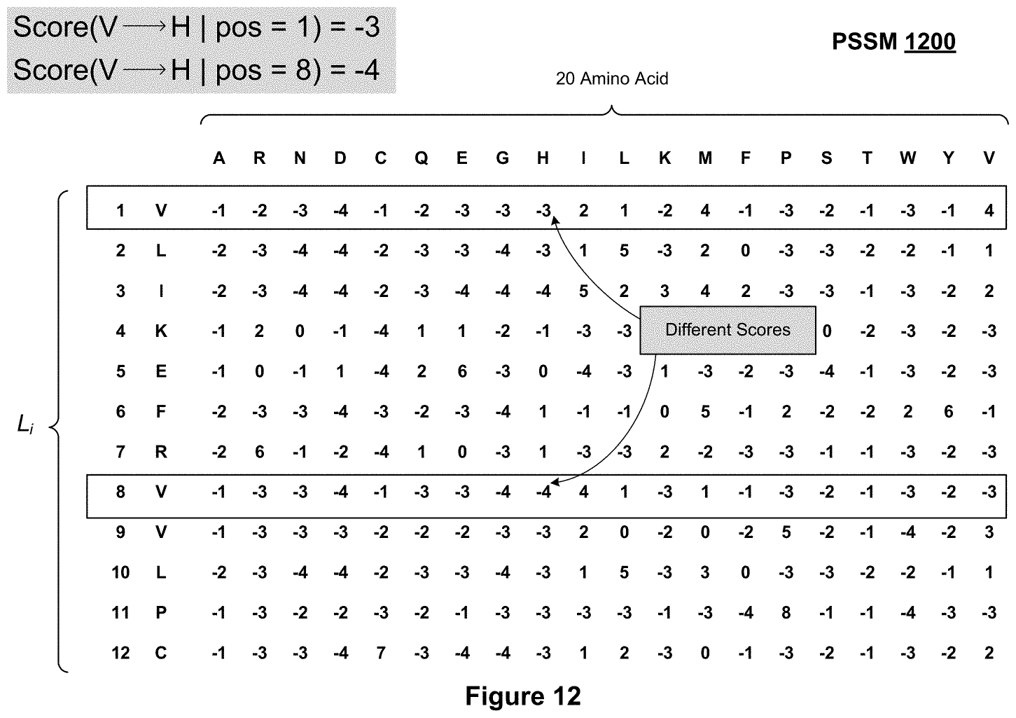

FIG. 10 shows one implementation of generating the PSFM and the PSSM. FIG. 11 illustrates an example PSFM 1100 encoding processed as input by the variant pathogenicity prediction network 190, in accordance with one implementation of the technology disclosed. FIG. 12 depicts an example PSSM 1200 encoding processed as input by the variant pathogenicity prediction network 190, in accordance with one implementation of the technology disclosed.

Given a query sequence, we first obtain its sequence profile by presenting it to PSI-BLAST to search and align homologous protein sequences from a protein database 1002 (e.g., Swiss-Prot Database). FIG. 10 shows the procedures of obtaining the sequence profile by using the PSI-BLAST program. The parameters h and j for PSI-BLAST are usually set to 0.001 and 3, respectively. The sequence profile of a protein encapsulates its homolog information pertaining to a query protein sequence. In PSI-BLAST, the homolog information is represented by two matrices: the PSFM and the PSSM. Examples of the PSFM and the PSSM are shown in FIGS. 11 and 12, respectively.

In FIG. 11, the (l, u)-th element (l ∈ {1, 2, . . . , Lᵢ}, u ∈ {1, 2, . . . , 20}) represents the chance of having the u-th amino acid in the l-th position of the query protein. For example, the chance of having the amino acid M in the 1^{st} position of the query protein is 0.36.

In FIG. 12, the (l, u)-th element (l ∈ {1, 2, . . . , Lᵢ}, u ∈ {1, 2, . . . , 20}) represents the likelihood score of the amino acid in the l-th position of the query protein being mutated to the u-th amino acid during the evolution process. For example, the score for the amino acid V in the 1^{st} position of the query protein being mutated to H during the evolution process is −3, while that in the 8^{th} position is −4.

### Coevolutionary Features Like CCMpred

Evolutionary coupling analysis (ECA) utilizes MSAs to identify correlation in changing (co-evolving) residue pairs, using the belief that residues in close proximity mutate in sync with the evolutionary functional and structural requirements of a protein. Popular ECA methods include: CCMPred, FreeContact, GREMLIN, PlmDCA, and PSICOV. These methods are useful for predicting long-range contacts in proteins with a high number of sequence homologues. In some implementations, the protein contact map generation sub-network 112 (or the variant pathogenicity prediction network 190) can be configured to take, as input, evolutionary coupling features generated from CCMPred, FreeContact, GREMLIN, PlmDCA, and/or PSICOV, and generate, as output, protein contact maps.

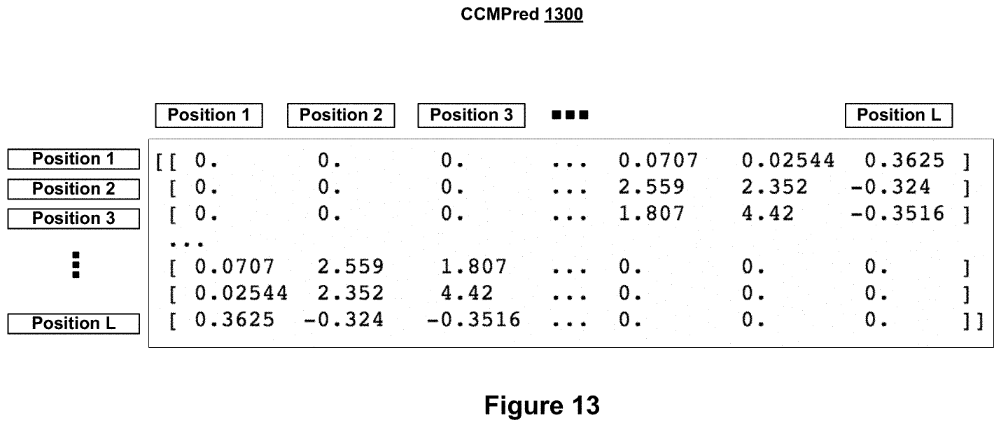

FIG. 13 shows an example CCMpred encoding 1300 processed as input by the variant pathogenicity prediction network 190, in accordance with one implementation of the technology disclosed. The CCMPred encoding 1300 is a predicted contact probability matrix with a dimensionality of sequence length (L)×sequence length (L). The CMMPred encoding 1300 includes coevolutionary contact probabilities/scores predicted using CCMPred. The CMMPred encoding 1300 distinguishes direct couplings between pairs of columns in a multiple sequence alignment from merely correlated pairs using pseudo-likelihood maximization (PLM).

### Tensorized Protein Data

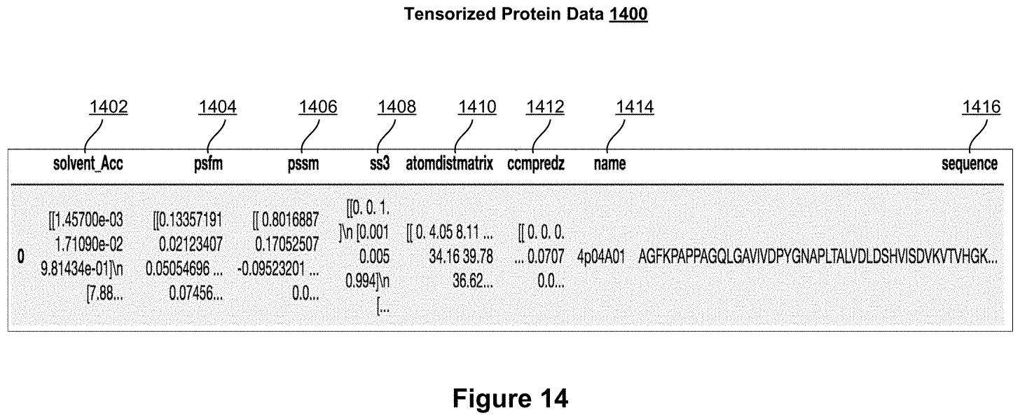

FIG. 14 illustrates an example of tensorized protein data 1400 processed as input by the variant pathogenicity prediction network 190, in accordance with one implementation of the technology disclosed. The tensorized protein data 1400 includes solve accessibility (SA) data 1402, PSFM data 1404, PSSM data 1406, secondary structure (SS) data 1408, atomic distance matrix 1410 (protein contact maps), and CCMPredz data 1412 (normalized CCMpred matrix (L*L)), in one implementation. The name 1414 of the protein and its amino acid sequence 1416 are also identified, in one implementation.

### 2D Protein Contact Maps as “Proxies” of 3D Protein Structures

Protein contact maps are two-dimensional (2D) representations of three-dimensional (3D) protein structures. A protein contact map forms a structural fingerprint of a protein and thus each protein can be identified based on its protein contact map. The protein contact map provides a host of useful information about the protein's 3D structure. For example, clusters of contacts represent certain secondary structures, and also capture non-local interactions, giving clues to the tertiary structure. The secondary structure, fold topology, and side-chain packing patterns can also be visualized conveniently and read from the contact map.

The shape of a protein is typically described using four levels of structural complexity: the primary, secondary, tertiary, and quaternary levels. For some proteins, a single polypeptide chain folded in its proper 3D structure creates the final protein. Protein structures are complex systems with several tens, hundreds or even thousands of residues, interacting with each other to help stabilize the tertiary structures so that specific functions can be realized in vivo. In this sense, the network modelling approach is suitable for characterizing and analyzing protein structures, in which residues correspond to vertices of the networks, and interaction (or any other type of relationship) between residues are represented as an edge linking the corresponding nodes. One way of conceptualizing and modelling protein structures is to consider the contacts between atoms in amino acids as a network of interactions, irrespective of secondary structures and fold type. There is a natural distinction of contacts into two types: long-range and short-range interactions. Long-range interactions occur between residues that are distant from each other in the primary structure but situated at a much closer distance in the tertiary structure. These interactions are important for defining the overall topology. Short-range interactions occur between residues that are local to each other in both the primary, secondary and tertiary structures. For most networks what is termed as a node and a link is fairly straightforward. When looking at protein transition states, the Cα atoms have been considered to be the nodes, and a link between two nodes is established if the atoms were within 8.5 Å of each other.

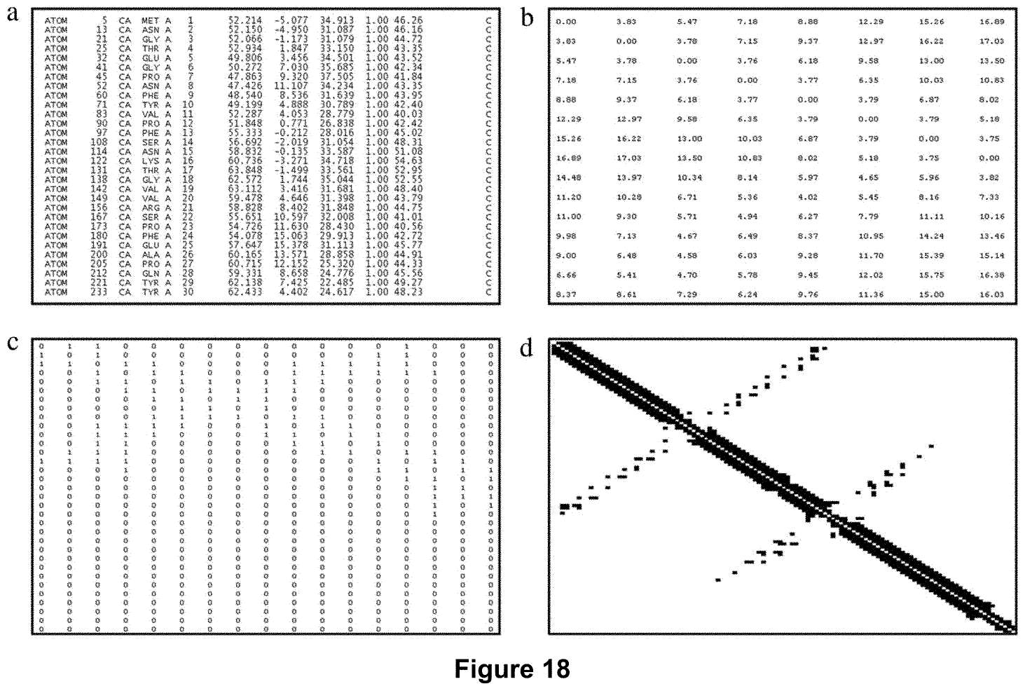

FIGS. 18(a)-(d) represent the steps in constructing the protein contact maps. The Cα atom of each amino acid has been considered as vertices of the corresponding protein contact network, as shown in FIG. 18(a). The distances between each pair of residues are determined using Euclidean distance and a part of the distance matrix is shown in FIG. 18(b). The diagonal line in the distance matrix is always zero since the distance between the same residues is zero. To determine whether any two residues are connected, the distance between the residues should be less than or equal to the cut-off value 7 Å distance, in the illustrated implementation. The choice of the cut-off distance is based on the range at which non-covalent interactions, which are responsible for the polypeptide chain to fold into its native-state. Various cut-offs ranging from 5 Å to 7 Å to 8.5 Å can be used. The protein contact map is derived using the said cut-off value represented in 2-dimensional binary matrix (FIG. 18(c)). If any two residues are connected, then the matrix cell values are set to 1 (black color) or else 0 (white color) if they are not connected (FIG. 18(d)).

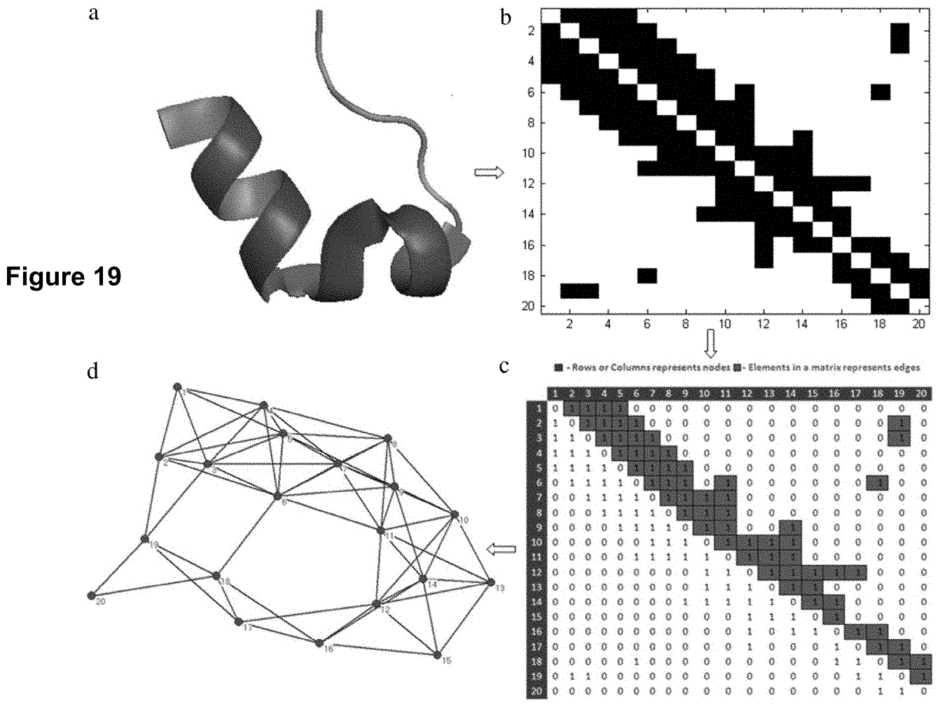

FIGS. 19(a)-(d) represent the relationship between a 2D protein contact map (FIG. 19(b)) and the corresponding 3D protein structure (FIG. 19(a)). For constructing a protein contact network (FIG. 19(d)) of the 3D protein structure (FIG. 19(a)), the Cartesian or xyz co-ordinates are required and these can be obtained from a RCSB protein data bank. The secondary structure of Trp-cage miniprotein (20 amino acids) is visualized using Rasmol, which is an open source molecular graphics visualization tool. The protein contact map is determined with the 7 Å cut-off distance, as shown in FIG. 19(b), and this distance denotes the non-covalent interactions. The protein contact network can be represented by its adjacency matrix (FIG. 19(c), i.e., binary depiction of the protein contact map). The rows or columns of the matrix denote the nodes or vertices and the elements in the matrix represent the links or edges. The elements aij in the matrix are equal to 1 whenever there is an edge connecting the vertices i and j, and equal to 0 otherwise. When the graph is undirected, the adjacency matrix is symmetric, i.e., the elements aij=aji for any i and j. Each element of the adjacency matrix represents a connection between two nodes. For instance, as the node 1 is connected to the nodes 2, 3, 4 and 5, we have a12=a13=a14=a15=1 and for the symmetric elements a21=a31=a41=a51=1. This adjacent matrix can then be visualized as an undirected network, as shown in FIG. 19(d), using Pajek, a program for large network analysis tool.

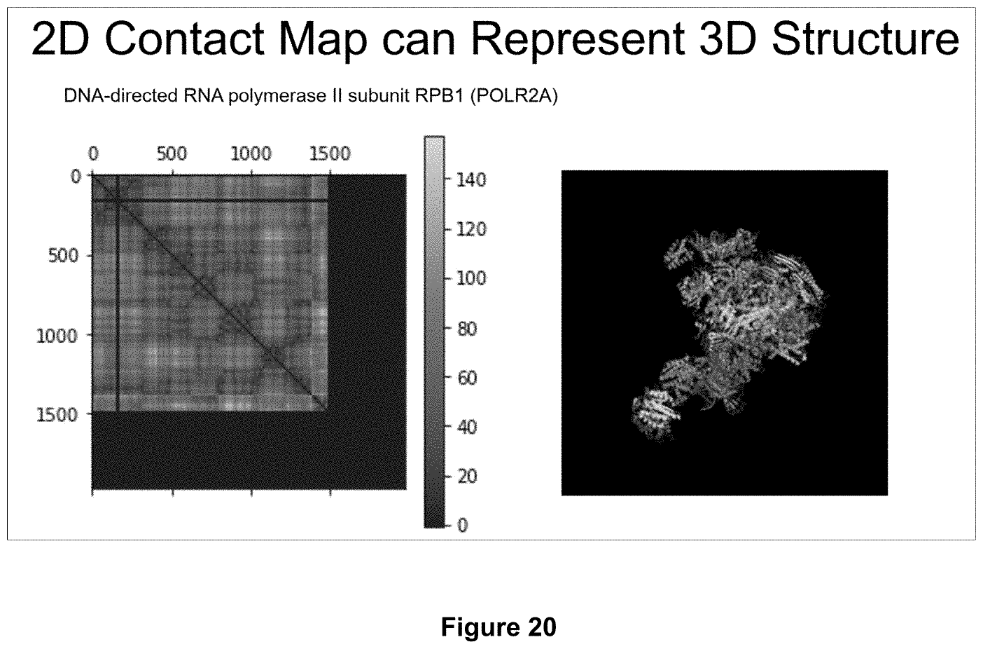

FIGS. 20, 21, 22, 23, 24, 25, and 26 illustrate different examples of 2D protein contact maps representing corresponding 3D protein structures.

In FIG. 20, a 3D protein structure of a protein is shown on the right, and a corresponding 2D protein contact map of the protein is shown on the left. The x and y axes of the 2D protein contact map are residues (amino acids) of the protein, i.e., L×L, where L=1500. The color coding of the 2D protein contact map indicates spatial proximity between pairs of residues. For example, those residue pairs of the protein that have a distance of 0 to 20 Angstroms (Ås) between them in the 3D protein structure are depicted with purple-colored contacts in the 2D protein contact map. Similarly, as another example, those residue pairs of the protein that have a distance of more than 140 Ås between them in the 3D protein structure are depicted with yellow-colored contacts in the 2D protein contact map.

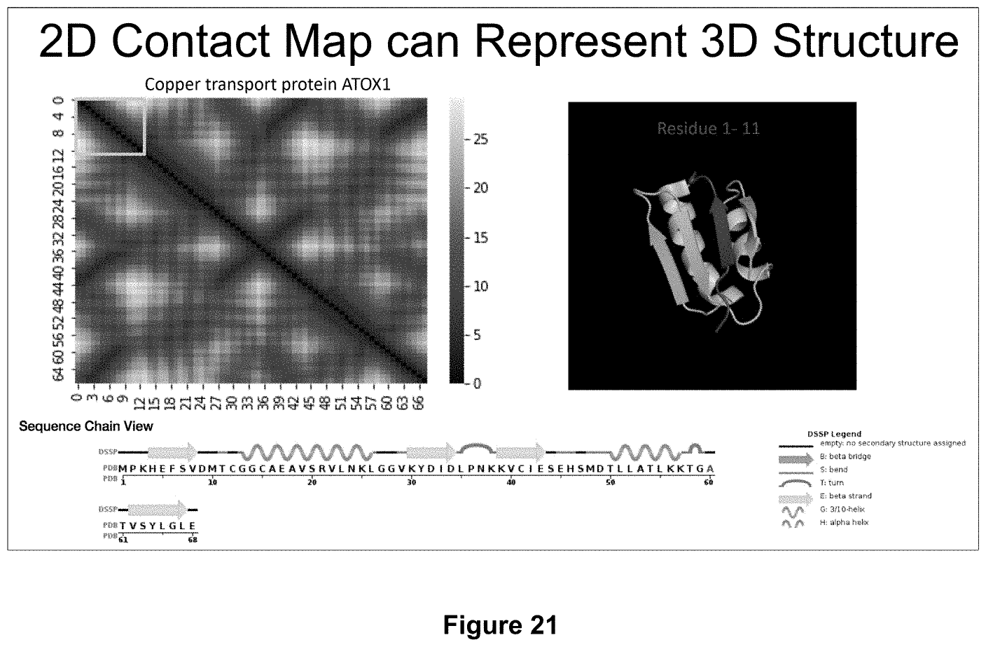

On the right, FIGS. 21 to 26 show a 3D protein structure of a copper transport protein ATOX1. On the left, FIGS. 21 to 26 show a 2D protein contact map corresponding to the 3D protein structure of the ATOX1 protein.

Note that, in FIGS. 21 to 26, contact values and resulting contact patterns are depicted by a color coding scheme. According to the color coding scheme, for example, those residue pairs of the ATOX1 protein that have a distance of 0 to 5 Ås between them in the 3D protein structure are depicted with black-colored contacts in the 2D protein contact map. Similarly, as another example, those residue pairs of the ATOX1 protein that have a distance of more than 25 Ås between them in the 3D protein structure are depicted with light orange-colored contacts in the 2D protein contact map.

In other words, in FIGS. 21 to 26, the 2D protein contact map depicts “spatially proximate” residues pairs in the 3D protein structure with “darker shades,” and depicts “spatially distant” residue pairs in the 3D protein structure with “lighter shades.” Also note that certain residue pairs may be “spatially distant” in the “sequential” amino acid sequence of the protein but may be “spatially proximate” in the 3D protein structure and therefore their “3D spatial proximity” is represented by “darker shades” in the 2D protein contact map.

Also note that the 2D protein contact map in FIGS. 21 to 26 has a dark diagonal. This is the case because the 2D protein contact map is a sequence length by sequence length matrix (i.e., L×L, where L=66), and each “coincident” instance of a residue pair of a same-position/same residue will result in a high contact value and therefore a dark contact pattern. So, for example, the 2D protein contact map will have high contact values and therefore dark contact patterns for residues pairs (1, 1), (2, 2), (3, 3), . . . , (66, 66), all of which fall on and form the dark diagonal in the 2D protein contact map.

FIG. 21 focuses on a region-of-interest that spans residues 1 to 11 of the ATOX1 protein. Residues 1 to 11 are located on a beta sheet/strand arrow of the 3D protein structure of the ATOX1 protein. This beta sheet arrow is depicted in red in FIG. 21, on the right.

On the left, in a cyan box, FIG. 21 highlights those contact values and resulting contact patterns in the 2D protein contact map that encode the spatial distances/interactions in the 3D protein structure of the ATOX1 protein between residue pairs spanning the residues 1 to 11. Inside the cyan box, the color shades of the contact values and the resulting contact patterns create a dark diagonal and lighter flanking regions around the dark diagonal. This indicates there is little to no 3D interaction between sequentially distant residue pairs spanning the residues 1 to 11. One exception though is residue pair (4, 8) or (8, 4). Even though residues 4 and 8 are sequentially distant, they have greater 3D spatial proximity/interaction, which is indicated by lighter shades corresponding to the contact values for the residue pair (4, 8) or (8, 4) in the cyan box in FIG. 21.

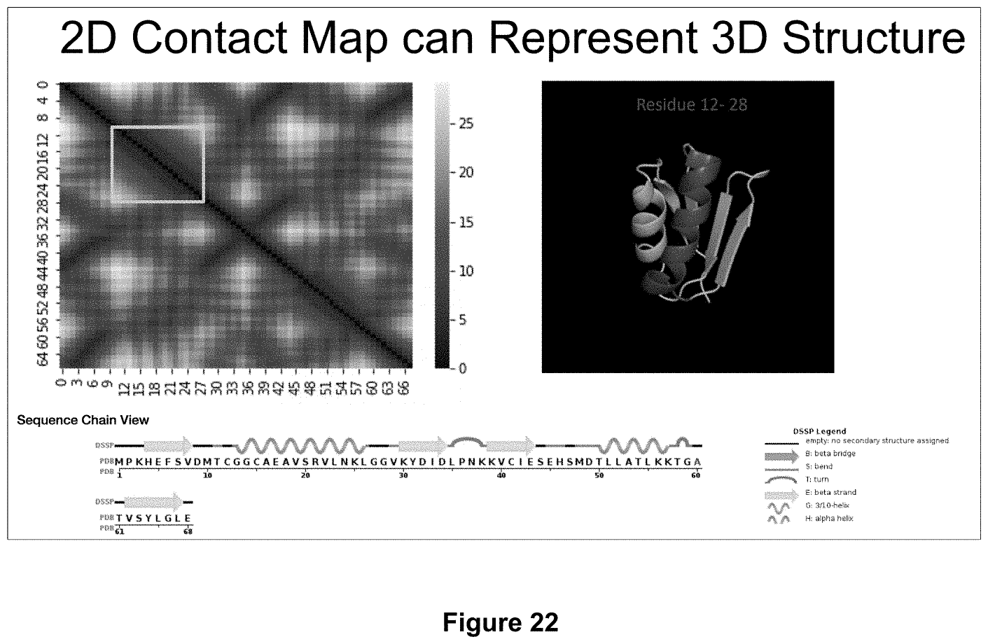

FIG. 22 focuses on a region-of-interest that spans residues 12 to 28 of the ATOX1 protein. Residues 12 to 28 are located on an alpha helix of the 3D protein structure of the ATOX1 protein. This alpha helix is depicted in red in FIG. 22, on the right.

On the left, in a cyan box, FIG. 22 highlights those contact values and resulting contact patterns in the 2D protein contact map that encode the spatial distances/interactions in the 3D protein structure of the ATOX1 protein between residue pairs spanning the residues 12 to 28. Inside the cyan box, the color shades of the contact values and the resulting contact patterns create an “expanded” dark diagonal and “shrunken” lighter flanking regions around the expanded dark diagonal. This indicates there is considerable 3D interaction between sequentially distant residue pairs spanning the residues 12 to 28. In particular, those residue pairs spanning the residues 12 to 28 that are four residue positions apart have greater interactions, for example, residue pairs (12, 16) or (16, 12), (20, 24) or (24, 20), and so on.

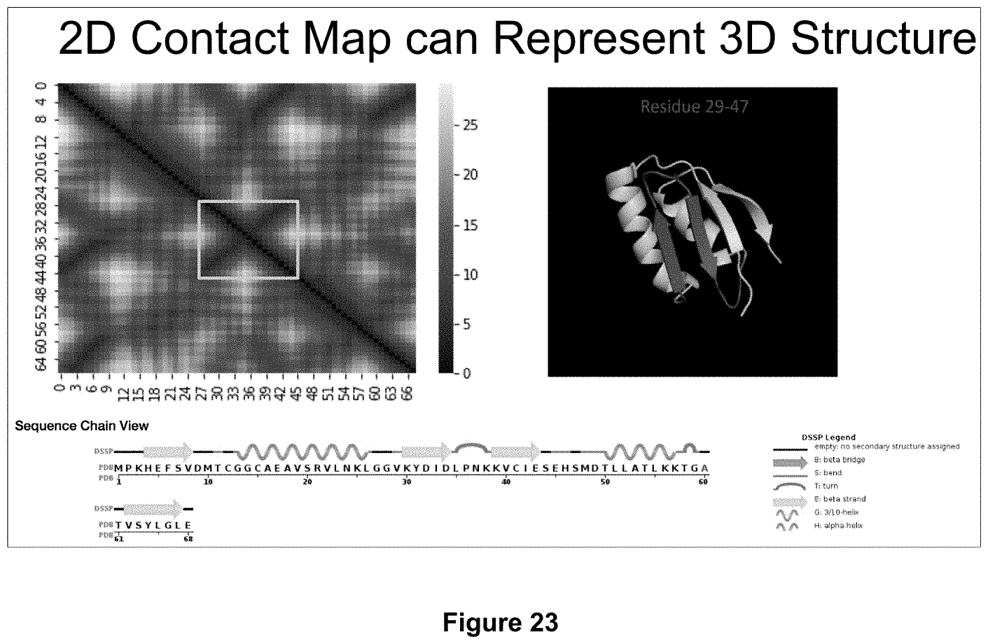

FIG. 23 focuses on a region-of-interest that spans residues 29 to 47 of the ATOX1 protein. Residues 29 to 47 are located on two anti-parallel beta sheet arrows of the 3D protein structure of the ATOX1 protein. These anti-parallel beta sheet arrows run in opposite directions and are depicted in red in FIG. 23, on the right.

On the left, in a cyan box, FIG. 23 highlights those contact values and resulting contact patterns in the 2D protein contact map that encode the spatial distances/interactions in the 3D protein structure of the ATOX1 protein between residue pairs spanning the residues 29 to 47. Inside the cyan box, the color shades of the contact values and the resulting contact patterns create a “cross” dark diagonal and “four triangles” lighter flanking regions around the cross dark diagonal. This indicates there is considerable 3D interaction between sequentially “inverse” residue pairs spanning the residues 29 to 47. For example, the sequentially adjacent residue pairs spanning the residues 29 to 47 are dark (e.g., residue pairs (29, 30) (30, 31)), but so are the sequentially opposite or inverse residue pairs (e.g., residue pairs (29, 47) and (28, 46).

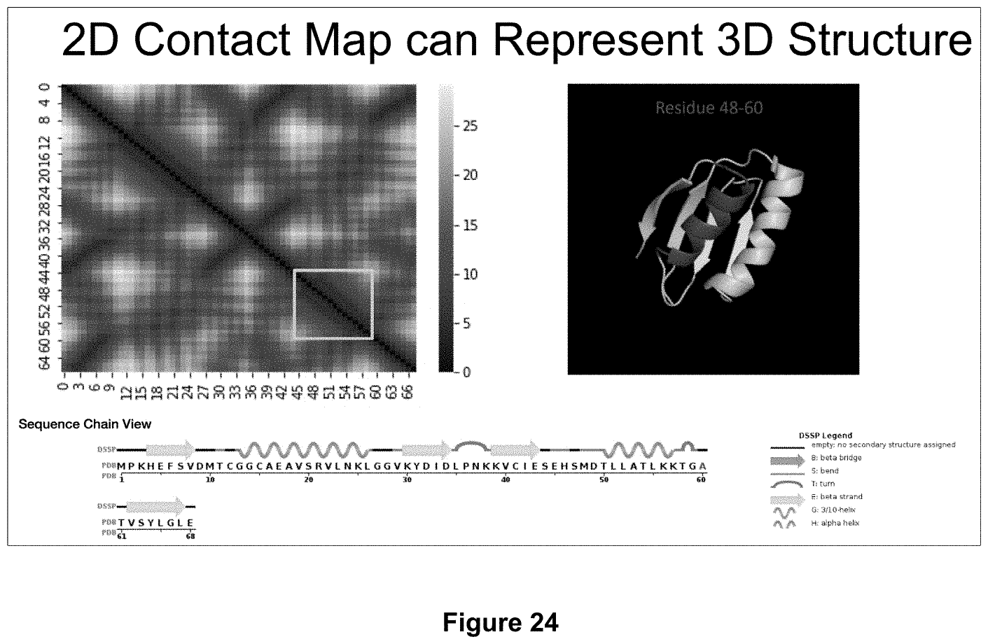

FIG. 24 focuses on a region-of-interest that spans residues 48 to 60 of the ATOX1 protein. Residues 48 to 60 are located on another alpha helix of the 3D protein structure of the ATOX1 protein. This alpha helix is depicted in red in FIG. 24, on the right.

On the left, in a cyan box, FIG. 24 highlights those contact values and resulting contact patterns in the 2D protein contact map that encode the spatial distances/interactions in the 3D protein structure of the ATOX1 protein between residue pairs spanning the residues 48 to 60. Inside the cyan box, the color shades of the contact values and the resulting contact patterns create another “expanded” dark diagonal and “shrunken” lighter flanking regions around the expanded dark diagonal. This indicates there is considerable 3D interaction between sequentially distant residue pairs spanning the residues 48 to 60. In particular, those residue pairs spanning the residues 48 to 60 that are four residue positions apart have greater interactions, for example, residue pairs (48, 52) or (52, 48), (56, 60) or (60, 56), and so on.

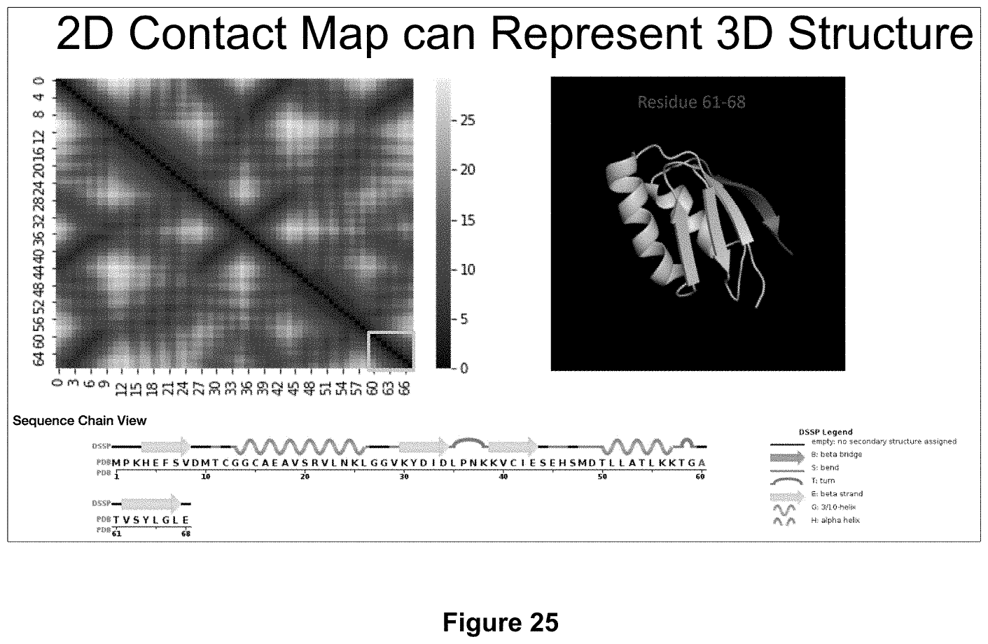

FIG. 25 focuses on a region-of-interest that spans residues 61 to 68 of the ATOX1 protein. Residues 61 to 68 are located on a small beta sheet/strand of the 3D protein structure of the ATOX1 protein. This small beta sheet is depicted in red in FIG. 25, on the right.

On the left, in a cyan box, FIG. 25 highlights those contact values and resulting contact patterns in the 2D protein contact map that encode the spatial distances/interactions in the 3D protein structure of the ATOX1 protein between residue pairs spanning the residues 61 to 68. Inside the cyan box, the color shades of the contact values and the resulting contact patterns create yet another “expanded” dark diagonal and “shrunken” lighter flanking regions around the expanded dark diagonal. This indicates there is considerable 3D interaction between sequentially distant residue pairs spanning the residues 61 to 68.

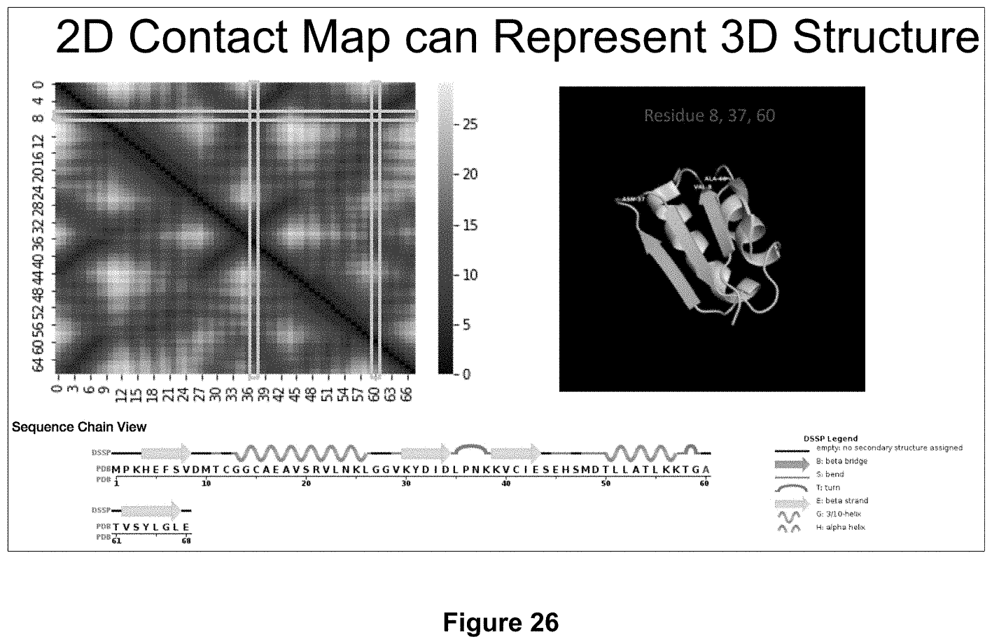

The cyan box in FIG. 26 shows considerable 3D spatial proximity/interaction between sequentially distant residue pairs (8, 37) and (8, 60) in the 2D protein contact map of the ATOX1 protein.

### 3D Protein Structures, and Therefore 2D Protein Contact Maps by Proxy, Contribute to Variant Pathogenicity Determination

The above discussion explained that 2D protein contact maps are proxies of 3D protein structures. Now the discussion turns to how the 3D protein structures, and therefore the 3D protein contact maps by proxy, contribute to variant pathogenicity determination

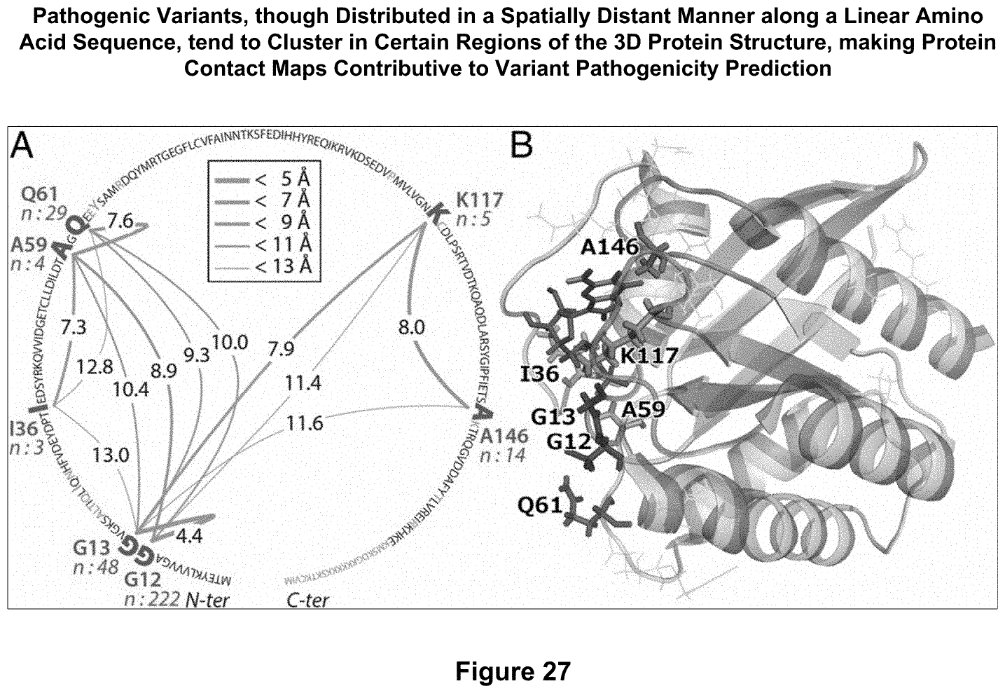

FIG. 27 graphically elucidates the notion that pathogenic variants, though distributed in a spatially distance manner along a linear/sequential amino acid sequence, tend to cluster in certain regions of the 3D protein structure, making protein contact maps contributive to the task of variant pathogenicity prediction. This means that protein contact maps are especially useful for determining pathogenicity of variants because protein contact maps capture 3D spatial proximity of sequentially distant residues that experience mutations in the 3D protein structure. Accordingly, the technology disclosed uses protein contact maps as input signals to generate variant pathogenicity predictions.

### Pathogenicity Classifier

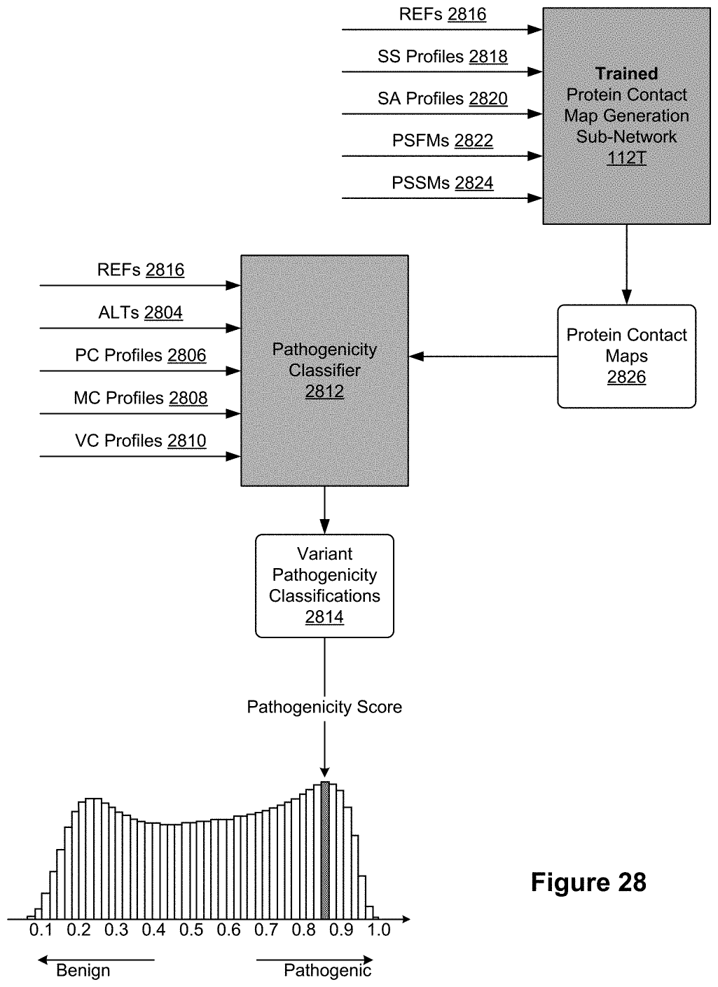

FIG. 28 depicts a pathogenicity classifier 2812 that makes variant pathogenicity classifications 2814 at least in part based on protein contact maps 2826 generated by the trained protein contact map generation sub-network 112T.

In one implementation, the pathogenicity classifier 2812 processes at least one of: (i) reference amino acid sequences (REFs) 2816 of proteins, (ii) alternative amino acid sequences 2804 of the proteins that contain variant amino acids caused by variant nucleotides, (iii) amino acid-wise primate conservation profiles 2806 of the proteins (e.g., PSFMs determined from alignment to only homologous primate sequences), (iv) amino acid-wise mammal conservation profiles 2808 of the proteins (e.g., PSFMs determined from alignment to only homologous mammal sequences), (v) amino acid-wise vertebrate conservation profiles 2816 of the proteins (e.g., PSFMs determined from alignment to only homologous vertebrate sequences), and (vi) the protein contact maps 2826. The resulting output produced by the pathogenicity classifier 2812 is the variant pathogenicity classifications 2814.

In one implementation, the trained protein contact map generation sub-network 112T generates the protein contact maps 2826 in response to processing at least one of: (i) the reference amino acid sequences (REFs) 2816 of the proteins, (ii) secondary structure (SS) profiles 2818 of the proteins, (iii) solvent accessibility (SA) profiles 2820 of the proteins, (iv) position-specific frequency matrices (PSFMs) 2822 of the proteins, and (v) position-specific scoring matrices (PSSMs) 2824 of the proteins.

In one implementation, the pathogenicity classifier 2812 is a neural network. In another implementation, the pathogenicity classifier 2812 uses convolutional neural networks (CNNs) with a plurality of convolution layers. In another implementation, the pathogenicity classifier 2812 uses recurrent neural networks (RNNs) such as a long short-term memory networks (LSTMs), bi-directional LSTMs (Bi-LSTMs), and gated recurrent units (GRU)s. In yet another implementation, the pathogenicity classifier 2812 uses both the CNNs and the RNNs. In yet another implementation, the pathogenicity classifier 2812 uses graph-convolutional neural networks that model dependencies in graph-structured data. In yet another implementation, the pathogenicity classifier 2812 uses variational autoencoders (VAEs). In yet another implementation, the pathogenicity classifier 2812 uses generative adversarial networks (GANs). In yet another implementation, the pathogenicity classifier 2812 can also be a language model based, for example, on self-attention such as the one implemented by Transformers and BERTs. In yet another implementation, the pathogenicity classifier 2812 uses a fully connected neural network (FCNN).

In yet other implementations, the pathogenicity classifier 2812 can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The pathogenicity classifier 2812 can use one or more loss functions such as logistic regression/log loss, multi-class cross-entropy/softmax loss, binary cross-entropy loss, L1 loss, L2 loss, smooth L1 loss, and Huber loss. It can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The pathogenicity classifier 2812 can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, attention mechanisms, and gaussian error linear unit.

The pathogenicity classifier 2812 can be trained using backpropagation-based gradient update techniques, in some implementations. Example gradient descent techniques that can be used for training the pathogenicity classifier 2812 include stochastic gradient descent (SGD), batch gradient descent, and mini-batch gradient descent. Some examples of gradient descent optimization algorithms that can be used to train the pathogenicity classifier 2812 are Momentum, Nesterov accelerated gradient, Adagrad, Adadelta, RMSprop, Adam, AdaMax, Nadam, and AMSGrad. In other implementations, the pathogenicity classifier 2812 can be trained by unsupervised learning, semi-supervised learning, self-learning, reinforcement learning, multitask learning, multimodal learning, transfer learning, knowledge distillation, and so on.

### Example Architecture of Pathogenicity Classifier

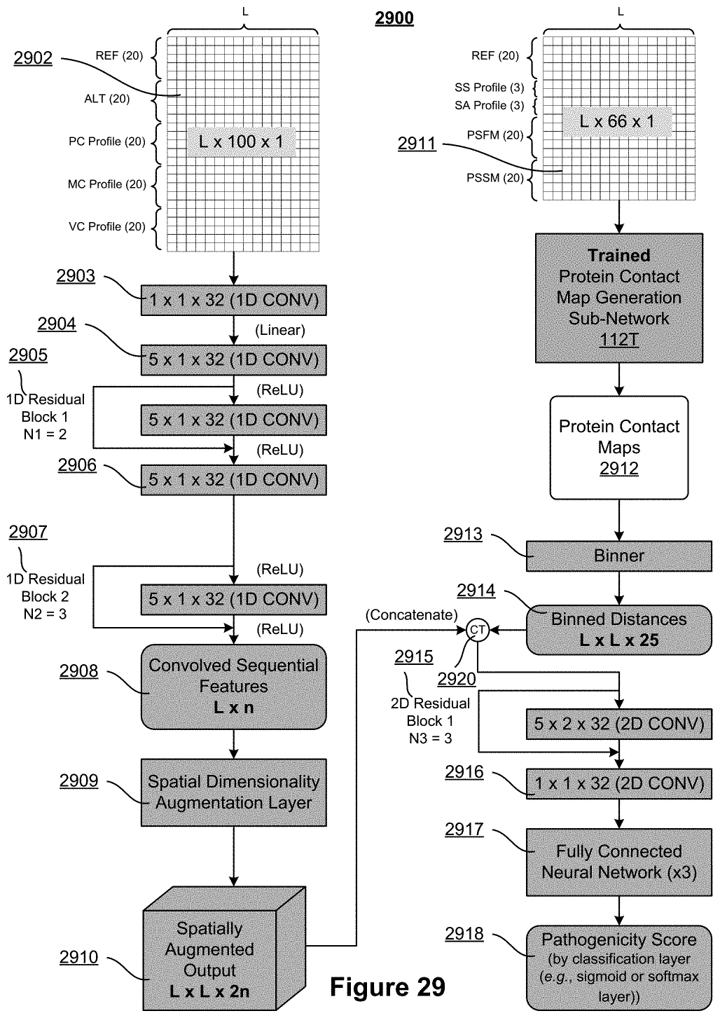

FIG. 29 depicts an example network architecture 2900 of the pathogenicity classifier 2812, in accordance with one implementation of the technology disclosed. In one implementation, the pathogenicity classifier 2812 comprises one or more initial 1D convolution layers 2903 and 2904, followed by a first 1D residual block 2905, followed by one or more intermediate 1D convolution layers (e.g., 1D convolution layer 2906), followed by a second 1D residual block 2907, followed by a spatial dimensionality augmentation layer 2909, followed by a first 2D residual block 2915, followed by one or more terminal 2D convolution layers (e.g., 1D convolution layer 2916), followed by a fully connected neural network 2917, and followed by a classification layer (e.g., sigmoid or softmax).

In FIG. 29, input 2911 to the trained protein contact map generation sub-network 112T is tensorized in a similar fashion as the input 202, as discussed above.

In FIG. 29, input 2902 to the pathogenicity classifier 2812 comprises a reference amino acid sequence of a protein-under-analysis, an alternative amino acid sequence of the protein-under-analysis that contains a variant amino acid caused by a variant nucleotide, an amino acid-wise primate conservation profile of the protein-under-analysis, an amino acid-wise mammal conservation profile of the protein-under-analysis, and an amino acid-wise vertebrate conservation profile of the protein-under-analysis. In one implementation, the input 2902 is a tensor that concatenates: (i) a L×20×1 matrix of a one-hot encoding of the reference amino acid sequence (where L is the number of amino acids in the reference amino acid sequence and 20 denotes the twenty amino acid categories), (ii) a L×20×1 matrix of a one-hot encoding of the alternative amino acid sequence, (iii) L×20×1 matrix of a PSFM determined from alignment to only homologous primate sequences, (iv) L×20×1 matrix of a PSFM determined from alignment to only homologous mammal sequences, and (v) L×20×1 matrix of a PSFM determined from alignment to only homologous vertebrate sequences. The resulting concatenated tensor 2902 is of size L×100×1, in accordance with some implementations.

The tensor 2902 is processed by the initial 1D convolution layers 2903 and 2904, the first 1D residual block 2905, the one or more intermediate 1D convolution layers (e.g., 1D convolution layer 2906), and the second 1D residual block 2907 to generate convolved sequential features 2908 (L×n). The spatial dimensionality augmentation layer 2909 processes the convolved sequential features 2908 and generates a spatially augmented output 2910 (L×L×2n).

The trained protein contact map generation sub-network 112T processes the input 2911 and generates protein contact maps 2912. A binner 2913 bins contact scores/distances in the protein contact maps 2912 into ranges of distances. For example, residue pair contact distances in the protein contact maps 2912 can be binned into 25 bins like [0-1 Å], [1-2 Å], [2-3 Å], [3-4 Å], [4-5 Å], [4-6 Å], [5-6 Å], . . . , [25 Å and above]. The output of the binner 2913 is binned distances 2914 of a dimensionality L×L×25.

The binned distances 2914 are concatenated (CT) 2920 with the spatially augmented output 2910. As used herein, concatenation operations can include combining by concatenation (stitching), summing, or multiplication. The resulting concatenated output is processed by the first 2D residual block 2915, the one or more terminal 2D convolution layers (e.g., 1D convolution layer 2916), the fully connected neural network 2917, and the classification layer (e.g., sigmoid or softmax (not shown)) to generate a pathogenicity score 2918.

Also note that in FIG. 29, “N1=2” denotes two 1D convolution layers inside the first 1D residual block 2905; “N2=3” denotes three 1D convolution layers inside the second 1D residual block 2907; and “N3=3” denotes three 2D convolution layers inside the first 2D residual block 2915. N1, N2, and N3 can be any numbers in different implementations.

### Processes

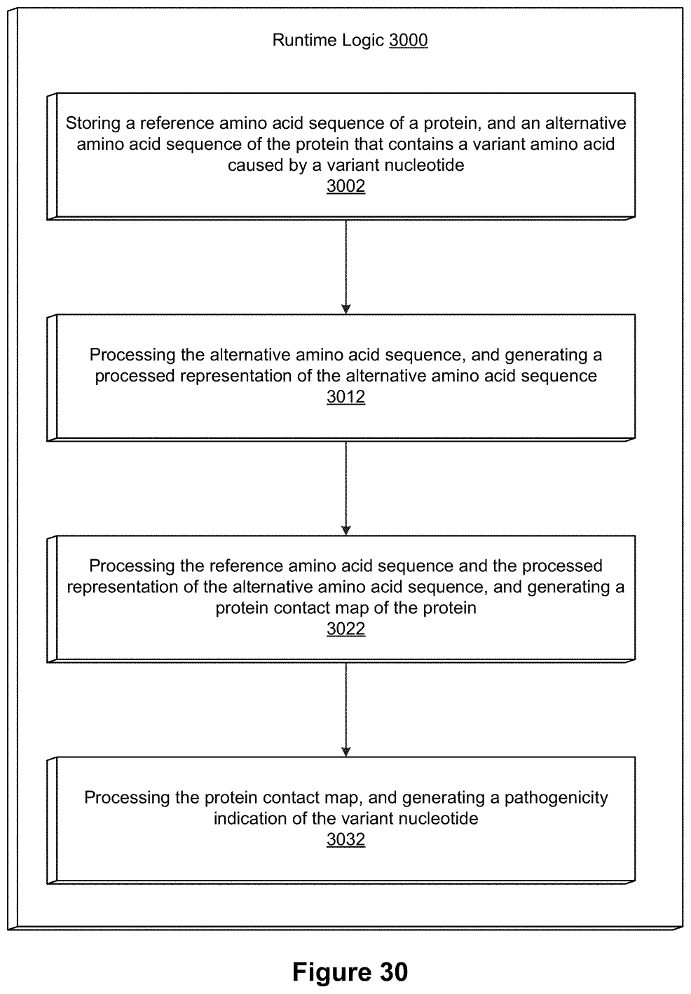

FIG. 30 is a flowchart that executes one implementation of a computer-implemented method of variant pathogenicity prediction. In one implementation, the flow chart of FIG. 30 is executed by runtime logic 3000. At step 3002, the method includes storing a reference amino acid sequence of a protein, and an alternative amino acid sequence of the protein that contains a variant amino acid caused by a variant nucleotide. At step 3012, the method includes processing the alternative amino acid sequence, and generating a processed representation of the alternative amino acid sequence. At step 3012, the method includes processing the reference amino acid sequence and the processed representation of the alternative amino acid sequence, and generating a protein contact map of the protein. At step 3032, the method includes processing the protein contact map, and generating a pathogenicity indication of the variant amino acid.

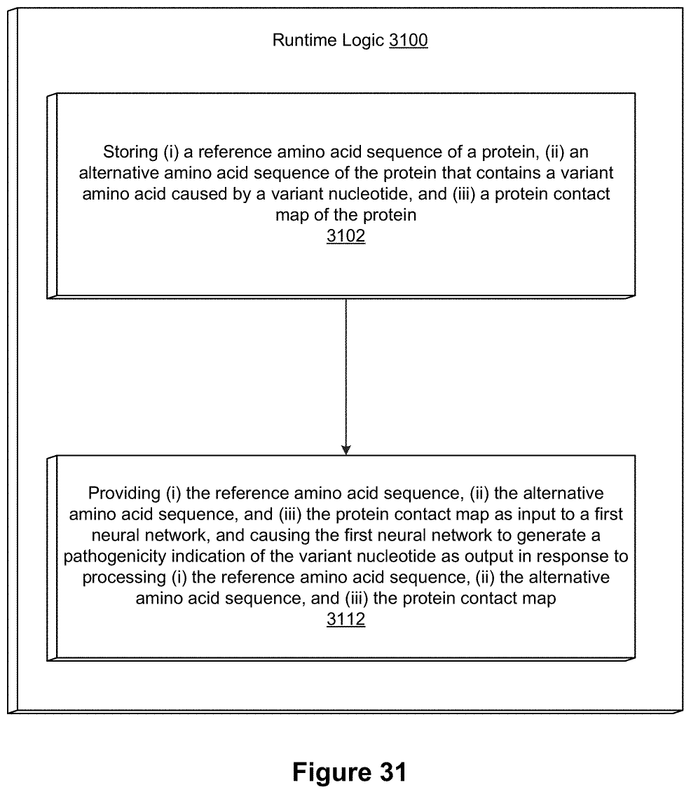

FIG. 31 is a flowchart that executes one implementation of a computer-implemented method of variant pathogenicity classification. In one implementation, the flow chart of FIG. 30 is executed by runtime logic 3100. At step 3102, the method includes storing (i) a reference amino acid sequence of a protein, (ii) an alternative amino acid sequence of the protein that contains a variant amino acid caused by a variant nucleotide, and (iii) a protein contact map of the protein. At step 3112, the method includes providing (i) the reference amino acid sequence, (ii) the alternative amino acid sequence, and (iii) the protein contact map as input to a first neural network, and causing the first neural network to generate a pathogenicity indication of the variant amino acid as output in response to processing (i) the reference amino acid sequence, (ii) the alternative amino acid sequence, and (iii) the protein contact map.

### Performance Results as Objective Indicia of Inventiveness and Non-Obviousness

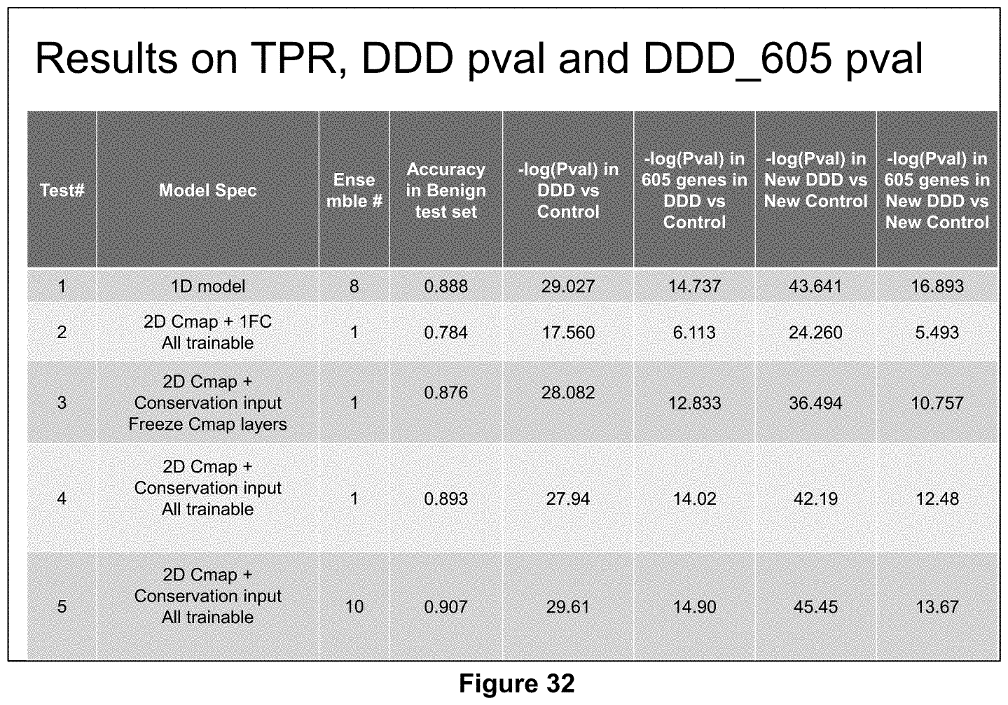

FIG. 32 shows performance results achieved by different implementations of the variant pathogenicity prediction network 190 on the task of variant pathogenicity prediction, as applied on different test data sets. The table in FIG. 32 shows performance evaluation of five models (rows) on five evaluation metrics (i.e., five test data sets) (columns)

The first model called “1D model” is a variant pathogenicity prediction network that uses only 1D convolutions and DOES NOT use 2D contact maps as part of its input. The 1D model can be considered the benchmark model for the purposes of this disclosure. Also note that, in FIG. 32, we are benchmarking with an ensemble of eight (8) 1D models.

The second model called “2D Cmap+1FC All trainable” is one implementation of the variant pathogenicity prediction network 190 with 2D convolutions and a fully connected (FC) neural network (e.g., the one depicted in FIG. 3 with the fully connected neural network 358 part of the pathogenicity scoring sub-network 144). “All trainable” refers to the notion that the entire variant pathogenicity prediction network 190, including the fully connected (FC) neural network, are retrained during the end-to-end retraining step of the transfer learning implementation (e.g., the transfer learning depicted in FIG. 1B).

The third model called “2D Cmap+Conservation Input Freeze Cmap Layers” is one implementation of the variant pathogenicity prediction network 190 with 2D convolutions and using as input conservation data (e.g., PSFMs, PSSMs, co-evolutionary features). “Freeze Cmap Layers” refers to the notion that those layers of the variant pathogenicity prediction network 190 that generate 2D contact maps as output (e.g., the protein contact map generation sub-network 112), are NOT retrained and kept frozen during the end-to-end retraining step of the transfer learning implementation (e.g., the transfer learning depicted in FIG. 1B). Note that the protein contact map generation sub-network 112 is trained at least once, as depicted in FIG. 1A, but in some implementations of transfer learning, not retrained in FIG. 1B as part of the variant pathogenicity prediction network 190. In other implementations of transfer learning, the protein contact map generation sub-network 112 can be retrained as part of the variant pathogenicity prediction network 190.

The fourth model called “2D Cmap+Conservation Input All trainable” is one implementation of the variant pathogenicity prediction network 190 with 2D convolutions and using as input conservation data (e.g., PSFMs, PSSMs, co-evolutionary features). “All trainable” refers to the notion that the entirety of the variant pathogenicity prediction network 190, including the variant encoding sub-network 128, the protein contact map generation sub-network 112, and the pathogenicity scoring sub-network 144, are retrained during the end-to-end retraining step of the transfer learning implementation (e.g., the transfer learning depicted in FIG. 1B).

The fifth model called “2D Cmap+Conservation Input All trainable” is one ENSEMBLE implementation of the variant pathogenicity prediction network 190 with 2D convolutions and using as input conservation data (e.g., PSFMs, PSSMs, co-evolutionary features). “Ensemble” refers to the notion that multiple instances of the variant pathogenicity prediction network 190 process the same input separately and produce respective outputs (e.g., respective pathogenicity predictions). A final output (e.g., a final pathogenicity prediction) is generated based on the respective outputs (e.g., by averaging the respective pathogenicity predictions, or by selecting a maximum one of the respective pathogenicity predictions). The multiple instances of the variant pathogenicity prediction network 190 have different coefficient/weight values but the same architecture. In the implementation illustrated in FIG. 32, the ensemble has ten (10) instances of the variant pathogenicity prediction network 190. “All trainable” refers to the notion that the entire variant pathogenicity prediction network 190 is retrained during the end-to-end retraining step of the transfer learning implementation (e.g., the transfer learning depicted in FIG. 1B).

Turning to the five evaluation metrics, the first evaluation metric “Accuracy in Benign test set” refers to the prediction accuracy of a given model on a data set of benign variants, for example, ten thousand (10,000) benign variants, which may include human benign variants and non-human primate benign variants (e.g., as discovered by PrimateAI).

The second evaluation metric “−log(Pval) in DDD vs Control” uses negative logarithm p-value (−log(Pval)) of a Wilcoxon rank-sum test to indicate the accuracy of a given model in identifying/separating pathogenic variants taken from individuals with developmental disabilities (DDD) like down syndrome as “pathogenic,” and identifying/separating benign variants taken from healthy individuals (Control) as “benign.”

The third evaluation metric “−log(Pval) in 605 genes in DDD vs Control” uses negative logarithm p-value (−log(Pval)) of a Wilcoxon rank-sum test to indicate the accuracy of a given model in identifying/separating pathogenic variants taken from individuals with developmental disabilities (DDD) like down syndrome and located on one of the “605 genes” clinically known to experience pathogenic variants as “pathogenic,” and identifying/separating benign variants taken from healthy individuals (Control) as “benign.”

The fourth evaluation metric “−log(Pval) in New DDD vs New Control” uses negative logarithm p-value (−log(Pval)) of a Wilcoxon rank-sum test to indicate the accuracy of a given model in identifying/separating pathogenic variants taken from new individuals with developmental disabilities (DDD) like down syndrome as “pathogenic,” and identifying/separating benign variants taken from new healthy individuals (Control) as “benign.”

The fifth evaluation metric “−log(Pval) in 605 genes in New DDD vs New Control” uses negative logarithm p-value (−log(Pval)) of a Wilcoxon rank-sum test to indicate the accuracy of a given model in identifying/separating pathogenic variants taken from new individuals with developmental disabilities (DDD) like down syndrome and located on one of the “605 genes” clinically known to experience pathogenic variants as “pathogenic,” and identifying/separating benign variants taken from new healthy individuals (Control) as “benign.”

Turning to the performance results of the five models on the five evaluation metrics (i.e., five test data sets), the fifth model, i.e., the “ENSEMBLE 2D Cmap+Conservation Input All trainable” model, outperforms all other models. This is demonstrated by 90.7% prediction accuracy of the fifth model in predicting benign variants in the 10,000 benign variant test data set as “benign,” and also by higher p-values. High p-values are indicative of a given model being better at separating/distinguishing pathogenic/disease-causing/deleterious DDD variants from the benign Control variants, thereby demonstrating better model performance.

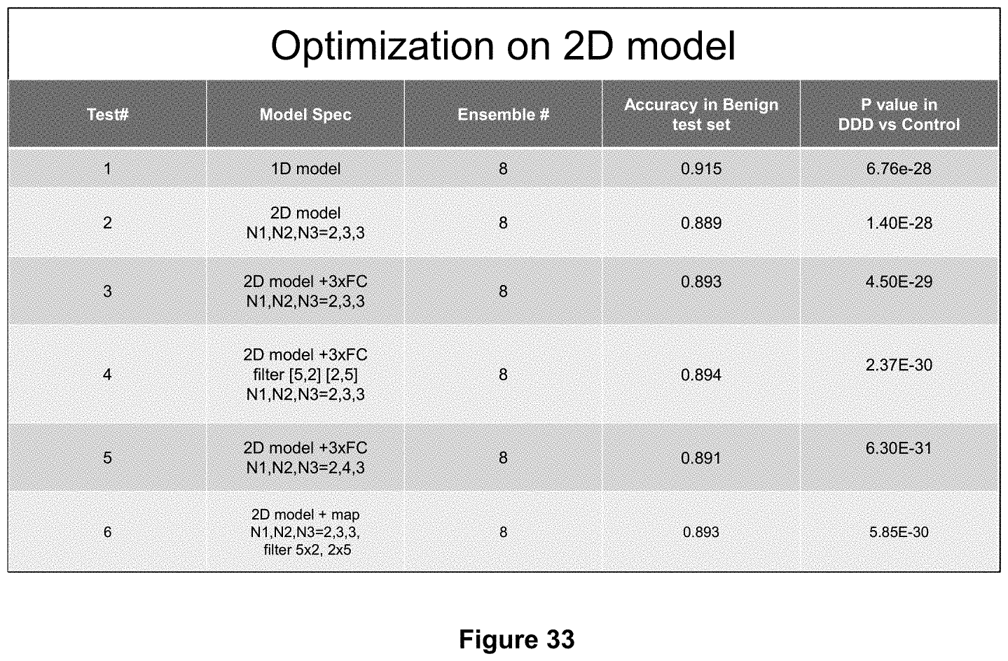

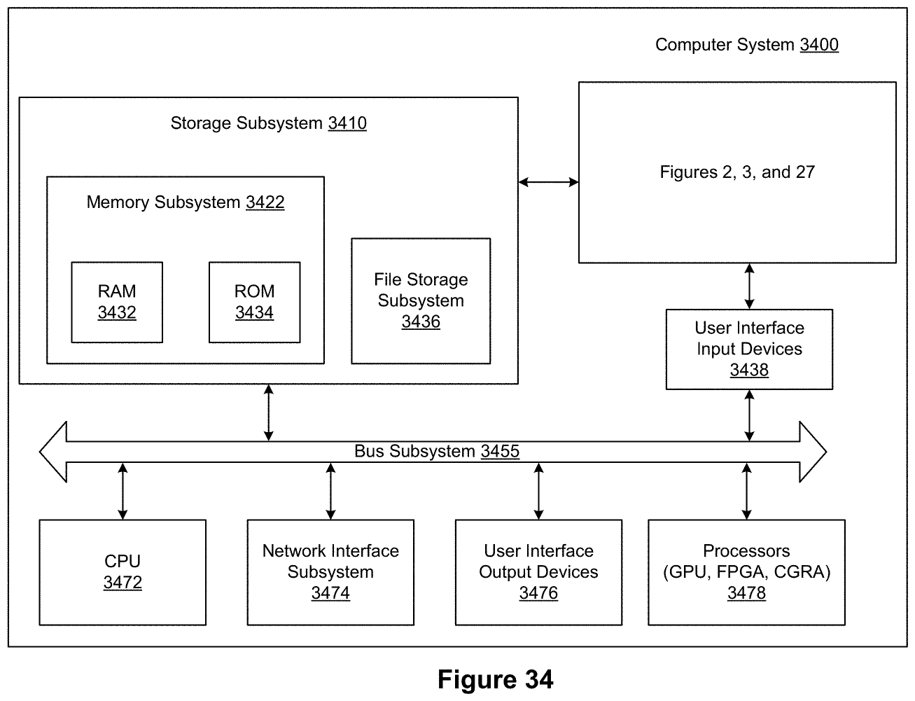

FIG. 33 shows performance results achieved by different implementations of the pathogenicity classifier on the task of variant pathogenicity classification, as applied on different test sets.

The table in FIG. 33 shows performance evaluation of six models (rows) on two evaluation metrics (i.e., two test data sets) (columns) Use of 2D contact maps (e.g., with the sixth model) is also evaluated against non-use with the 2D models.

The first test data set “Accuracy in Benign test set” is a data set of benign variants, for example, ten thousand (10,000) benign variants, which may include human benign variants and non-human primate benign variants (e.g., as discovered by PrimateAI). The second test data set “−log(Pval) in DDD vs Control” uses negative logarithm p-value (−log(Pval)) of a Wilcoxon rank-sum test to indicate the accuracy of a given model in identifying/separating pathogenic variants taken from individuals with developmental disabilities (DDD) like down syndrome as “pathogenic,” and identifying/separating benign variants taken from healthy individuals (Control) as “benign.” Also note that, in FIG. 33, each of the six models are implemented as an ensemble of eight (8) instances. In other implementations, different number of instances can be used.

The first model called “1D model” is a variant pathogenicity prediction network that uses only 1D convolutions and DOES NOT use 2D contact maps as part of its input. The 1D model can be considered the benchmark model for the purposes of this disclosure.

The five 2D models (rows 2 to 6), i.e., the five different implementations of the pathogenicity classifier 2812, differ in their respective architectures with different numbers of residual blocks in different residual block sets N1, N2, and N3, use of fully connected layers against non-use, and use of different filter sizes (e.g., 5×2 v/s 2×5).

As seen in FIG. 33, the pathogenicity classifier 2812 that uses the 2D contact maps as input features, i.e., the sixth model, has better performance on average.

### Computer System

FIG. 34 is an example computer system 3400 that can be used to implement the technology disclosed. Computer system 3400 includes at least one central processing unit (CPU) 3472 that communicates with a number of peripheral devices via bus subsystem 3455. These peripheral devices can include a storage subsystem 3410 including, for example, memory devices and a file storage subsystem 3436, user interface input devices 3438, user interface output devices 3476, and a network interface subsystem 3474. The input and output devices allow user interaction with computer system 3400. Network interface subsystem 3474 provides an interface to outside networks, including an interface to corresponding interface devices in other computer systems.

In one implementation, the pathogenicity classifier 2104 is communicably linked to the storage subsystem 3410 and the user interface input devices 3438.

User interface input devices 3438 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 3400.

User interface output devices 3476 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 3400 to the user or to another machine or computer system.

Storage subsystem 3410 stores programming and data constructs that provide the functionality of some or all of the modules and methods described herein. These software modules are generally executed by processors 3478.

Processors 3478 can be graphics processing units (GPUs), field-programmable gate arrays (FPGAs), application-specific integrated circuits (ASICs), and/or coarse-grained reconfigurable architectures (CGRAs). Processors 3478 can be hosted by a deep learning cloud platform such as Google Cloud Platform™, Xilinx™, and Cirrascale™. Examples of processors 3478 include Google's Tensor Processing Unit (TPU)™, rackmount solutions like GX4 Rackmount Series™, GX34 Rackmount Series™, NVIDIA DGX-1™, Microsoft' Stratix V FPGA™, Graphcore's Intelligent Processor Unit (IPU)™, Qualcomm's Zeroth Platform™ with Snapdragon processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 3422 used in the storage subsystem 3410 can include a number of memories including a main random access memory (RAM) 3432 for storage of instructions and data during program execution and a read only memory (ROM) 3434 in which fixed instructions are stored. A file storage subsystem 3436 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 3436 in the storage subsystem 3410, or in other machines accessible by the processor.

Bus subsystem 3455 provides a mechanism for letting the various components and subsystems of computer system 3400 communicate with each other as intended. Although bus subsystem 3455 is shown schematically as a single bus, alternative implementations of the bus subsystem can use multiple busses.

Computer system 3400 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 3400 depicted in FIG. 34 is intended only as a specific example for purposes of illustrating the preferred implementations of the present invention. Many other configurations of computer system 3400 are possible having more or less components than the computer system depicted in FIG. 34.

“Logic”, as used herein, can be implemented in the form of a computer product including a non-transitory computer readable storage medium with computer usable program code for performing the method steps described herein. The “logic” can be implemented in the form of an apparatus including a memory and at least one processor that is coupled to the memory and operative to perform exemplary method steps. The “logic” can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media). In one implementation, the logic implements a data processing function. The logic can be a general purpose, single core or multicore, processor with a computer program specifying the function, a digital signal processor with a computer program, configurable logic such as an FPGA with a configuration file, a special purpose circuit such as a state machine, or any combination of these. Also, a computer program product can embody the computer program and configuration file portions of the logic.

### Clauses

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed or elements thereof can be implemented in the form of a computer product, including a non-transitory computer readable storage medium with computer usable program code for performing the method steps indicated. Furthermore, one or more implementations and clauses of the technology disclosed or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:

### Clauses Set 1

1. A variant pathogenicity prediction network, comprising:
memory storing a reference amino acid sequence of a protein, and an alternative amino acid sequence of the protein that contains a variant amino acid caused by a variant nucleotide;
a variant encoding sub-network, having access to the memory, configured to process the alternative amino acid sequence, and generate a processed representation of the alternative amino acid sequence;
a protein contact map generation sub-network, in communication with the variant encoding sub-network, configured to process the reference amino acid sequence and the processed representation of the alternative amino acid sequence, and generate a protein contact map of the protein; and
a pathogenicity scoring sub-network, in communication with the protein contact map generation sub-network, configured to process the protein contact map, and generate a pathogenicity indication of the variant amino acid.
2. The variant pathogenicity prediction network of clause 1, wherein the memory further stores an amino acid-wise primate conservation profile of the protein, and
wherein the processed representation of the alternative amino acid sequence is generated by the variant encoding sub-network in response to processing the alternative amino acid sequence and the amino acid-wise primate conservation profile.
3. The variant pathogenicity prediction network of any of clauses 1-2, wherein the memory further stores an amino acid-wise mammal conservation profile of the protein, and
wherein the processed representation of the alternative amino acid sequence is generated by the variant encoding sub-network in response to processing the alternative amino acid sequence and the amino acid-wise mammal conservation profile.
4. The variant pathogenicity prediction network of any of clauses 1-3, wherein the memory further stores an amino acid-wise vertebrate conservation profile of the protein, and
wherein the processed representation of the alternative amino acid sequence is generated by the variant encoding sub-network in response to processing the alternative amino acid sequence and the amino acid-wise vertebrate conservation profile.
5. The variant pathogenicity prediction network of any of clauses 1-4, wherein the processed representation of the alternative amino acid sequence is generated by the variant encoding sub-network in response to processing the alternative amino acid sequence, the amino acid-wise primate conservation profile, the amino acid-wise mammal conservation profile, and the amino acid-wise vertebrate conservation profile.
6. The variant pathogenicity prediction network of any of clauses 1-5, wherein the processed representation of the alternative amino acid sequence is generated by the variant encoding sub-network in response to processing the alternative amino acid sequence, the amino acid-wise primate conservation profile, and the amino acid-wise mammal conservation profile.
7. The variant pathogenicity prediction network of any of clauses 1-6, wherein the processed representation of the alternative amino acid sequence is generated by the variant encoding sub-network in response to processing the alternative amino acid sequence, the amino acid-wise primate conservation profile, and the amino acid-wise vertebrate conservation profile.
8. The variant pathogenicity prediction network of any of clauses 1-7, wherein the processed representation of the alternative amino acid sequence is generated by the variant encoding sub-network in response to processing the alternative amino acid sequence, the amino acid-wise mammal conservation profile, and the amino acid-wise vertebrate conservation profile.
9. The variant pathogenicity prediction network of any of clauses 1-8, wherein the memory further stores an amino acid-wise secondary structure profile of the protein, and
wherein the protein contact map of the protein is generated by the protein contact map generation sub-network in response to processing the reference amino acid sequence and the amino acid-wise secondary structure profile.
10. The variant pathogenicity prediction network of any of clauses 1-9, wherein the memory further stores an amino acid-wise solvent accessibility profile of the protein, and
wherein the protein contact map of the protein is generated by the protein contact map generation sub-network in response to processing the reference amino acid sequence and the amino acid-wise solvent accessibility profile.
11. The variant pathogenicity prediction network of any of clauses 1-10, wherein the memory further stores an amino acid-wise position-specific frequency matrix of the protein, and
wherein the protein contact map of the protein is generated by the protein contact map generation sub-network in response to processing the reference amino acid sequence and the amino acid-wise position-specific frequency matrix.
12. The variant pathogenicity prediction network of any of clauses 1-11, wherein the memory further stores an amino acid-wise position-specific scoring matrix of the protein, and
wherein the protein contact map of the protein is generated by the protein contact map generation sub-network in response to processing the reference amino acid sequence and the amino acid-wise position-specific scoring matrix.
13. The variant pathogenicity prediction network of any of clauses 1-12, wherein the protein contact map of the protein is generated by the protein contact map generation sub-network in response to processing the reference amino acid sequence, the amino acid-wise secondary structure profile, the amino acid-wise solvent accessibility profile, the amino acid-wise position-specific frequency matrix, and the amino acid-wise position-specific scoring matrix.
14. The variant pathogenicity prediction network of any of clauses 1-13, wherein the protein contact map of the protein is generated by the protein contact map generation sub-network in response to processing the reference amino acid sequence, the amino acid-wise secondary structure profile, and the amino acid-wise solvent accessibility profile.
15. The variant pathogenicity prediction network of any of clauses 1-14, wherein the protein contact map of the protein is generated by the protein contact map generation sub-network in response to processing the reference amino acid sequence, the amino acid-wise secondary structure profile, and the amino acid-wise position-specific frequency matrix.
16. The variant pathogenicity prediction network of any of clauses 1-15, wherein the protein contact map of the protein is generated by the protein contact map generation sub-network in response to processing the reference amino acid sequence, the amino acid-wise secondary structure profile, and the amino acid-wise position-specific scoring matrix.
17. The variant pathogenicity prediction network of any of clauses 1-16, wherein the protein contact map of the protein is generated by the protein contact map generation sub-network in response to processing the reference amino acid sequence, the amino acid-wise solvent accessibility profile, and the amino acid-wise position-specific frequency matrix.
18. The variant pathogenicity prediction network of any of clauses 1-17, wherein the protein contact map of the protein is generated by the protein contact map generation sub-network in response to processing the reference amino acid sequence, the amino acid-wise solvent accessibility profile, and the amino acid-wise position-specific scoring matrix.
19. The variant pathogenicity prediction network of any of clauses 1-18, wherein the protein contact map of the protein is generated by the protein contact map generation sub-network in response to processing the reference amino acid sequence, the amino acid-wise position-specific frequency matrix, and the amino acid-wise position-specific scoring matrix.
20. The variant pathogenicity prediction network of any of clauses 1-19, wherein the protein contact map of the protein is generated by the protein contact map generation sub-network in response to processing the reference amino acid sequence, the amino acid-wise secondary structure profile, the amino acid-wise solvent accessibility profile, and the amino acid-wise position-specific frequency matrix.
21. The variant pathogenicity prediction network of any of clauses 1-20, wherein the protein contact map of the protein is generated by the protein contact map generation sub-network in response to processing the reference amino acid sequence, the amino acid-wise secondary structure profile, the amino acid-wise solvent accessibility profile, and the amino acid-wise position-specific scoring matrix.
22. The variant pathogenicity prediction network of any of clauses 1-21, wherein the processed representation of the alternative amino acid sequence is provided as input to a first layer of the protein contact map generation sub-network.
23. The variant pathogenicity prediction network of any of clauses 1-22, wherein the processed representation of the alternative amino acid sequence is provided as input to one or more intermediate layers of the protein contact map generation sub-network.
24. The variant pathogenicity prediction network of any of clauses 1-23, wherein the processed representation of the alternative amino acid sequence is provided as input to a final layer of the protein contact map generation sub-network.
25. The variant pathogenicity prediction network of any of clauses 1-24, wherein the processed representation of the alternative amino acid sequence is combined (e.g., concatenated, summed) with an input to the protein contact map generation sub-network.
26. The variant pathogenicity prediction network of any of clauses 1-25, wherein the processed representation of the alternative amino acid sequence is combined (e.g., concatenated, summed) with one or more intermediate outputs of the protein contact map generation sub-network.
27. The variant pathogenicity prediction network of any of clauses 1-26, wherein the processed representation of the alternative amino acid sequence is combined (e.g., concatenated, summed) with a final output of the protein contact map generation sub-network.
28. The variant pathogenicity prediction network of any of clauses 1-27, wherein the reference amino acid sequence has L amino acids.
29. The variant pathogenicity prediction network of any of clauses 1-28, wherein the reference amino acid sequence is characterized as a one-hot encoded matrix of size L by C, where C denotes twenty amino acid categories.
30. The variant pathogenicity prediction network of any of clauses 1-29, wherein the amino acid-wise primate conservation profile is of size L by C.
31. The variant pathogenicity prediction network of any of clauses 1-30, wherein the amino acid-wise mammal conservation profile is of size L by C.
32. The variant pathogenicity prediction network of any of clauses 1-31, wherein the amino acid-wise vertebrate conservation profile is of size L by C.
33. The variant pathogenicity prediction network of any of clauses 1-32, wherein the amino acid-wise secondary structure profile is characterized as a three-state encoded matrix of size L by S, where S denotes three secondary structure states.
34. The variant pathogenicity prediction network of any of clauses 1-33, wherein the amino acid-wise solvent accessibility profile is characterized as a three-state encoded matrix of size L by A, where A denotes three solvent accessibility states.
35. The variant pathogenicity prediction network of any of clauses 1-34, wherein the amino acid-wise position-specific scoring matrix is of size L by C.
36. The variant pathogenicity prediction network of any of clauses 1-35, wherein the amino acid-wise position-specific frequency matrix is of size L by C.
37. The variant pathogenicity prediction network of any of clauses 1-36, wherein the variant encoding sub-network is a first convolutional neural network.
38. The variant pathogenicity prediction network of any of clauses 1-37, wherein the first convolutional neural network comprises one or more one-dimensional (1D) convolution layers.
39. The variant pathogenicity prediction network of any of clauses 1-38, wherein the protein contact map generation sub-network is a second convolutional neural network.
40. The variant pathogenicity prediction network of any of clauses 1-39, wherein the second convolutional neural network comprises (i) one or more 1D convolution layers, followed by (ii) one or more residual blocks with 1D convolutions, followed by (iii) a spatial dimensionality augmentation layer, followed by (iv) one or more residual blocks with two-dimensional (2D) convolutions, and followed by (v) one or more 2D convolution layers.
41. The variant pathogenicity prediction network of any of clauses 1-40, wherein a spatial dimensionality (e.g., width×height) of an input processed by a first 1D convolution layer in the one or more 1D convolution layers of the second convolutional neural network is L by 1.
42. The variant pathogenicity prediction network of any of clauses 1-41, wherein a depth dimensionality of the input processed by the first 1D convolution layer is D (e.g., 66), where D=C+S+A+C+C.
43. The variant pathogenicity prediction network of any of clauses 1-42, wherein an output of a final residual block in the one or more residual blocks with 1D convolutions of the second convolutional neural network is processed by the spatial dimensionality augmentation layer to generate a spatially augmented output.
44. The variant pathogenicity prediction network of any of clauses 1-43, wherein a spatial dimensionality of the spatially augmented output is L by L.
45. The variant pathogenicity prediction network of any of clauses 1-44, wherein the spatial dimensionality augmentation layer is configured to apply an outer product on the output of the final residual block to generate the spatially augmented output.
46. The variant pathogenicity prediction network of any of clauses 1-45, wherein the spatially augmented output is processed by a first residual block in the one or more residual blocks with 2D convolutions of the second convolutional neural network.
47. The variant pathogenicity prediction network of any of clauses 1-46, wherein a total dimensionality of the protein contact map generated by a final 2D convolution layer in the one or more 2D convolution layers of the second convolutional neural network is L by L by 1.
48. The variant pathogenicity prediction network of any of clauses 1-47, wherein the protein contact map generation sub-network is pre-trained on reference amino acid sequences of bacteria proteins with known protein contact maps.
49. The variant pathogenicity prediction network of any of clauses 1-48, wherein the protein contact map generation sub-network is pre-trained using a mean squared error loss function that minimizes error between known protein contact maps and protein contact maps predicted by the protein contact map generation sub-network during the pre-training.
50. The variant pathogenicity prediction network of any of clauses 1-49, wherein the protein contact map generation sub-network is pre-trained using a mean absolute error loss function that minimizes error between the known protein contact maps and protein contact maps predicted by the protein contact map generation sub-network during the pre-training.
51. The variant pathogenicity prediction network of any of clauses 1-50, wherein the protein contact map generation sub-network is pre-trained to generate the protein contact map as output in response to processing the reference amino acid sequence and at least one of the amino acid-wise secondary structure profile, the amino acid-wise solvent accessibility profile, the amino acid-wise position-specific scoring matrix, and the amino acid-wise position-specific frequency matrix.
52. The variant pathogenicity prediction network of any of clauses 1-51, wherein the pathogenicity scoring sub-network is jointly trained end-to-end with the pre-trained protein contact map generation sub-network and the variant encoding sub-network to generate the pathogenicity indication of the variant amino acid as output in response to processing the protein contact map, and
wherein the protein contact map is generated by the pre-trained protein contact map generation sub-network in response to processing:
   the reference amino acid sequence and at least one of the amino acid-wise secondary structure profile, the amino acid-wise solvent accessibility profile, the amino acid-wise position-specific scoring matrix, and the amino acid-wise position-specific frequency matrix, and
   a processed representation generated by the variant encoding sub-network in response to processing the alternative amino acid sequence and at least one of the amino acid-wise primate conservation profile, the amino acid-wise mammal conservation profile, and the amino acid-wise vertebrate conservation profile.
53. The variant pathogenicity prediction network of any of clauses 1-52, wherein the pre-trained protein contact map generation sub-network is kept frozen and not retrained during training of the variant encoding sub-network and the pathogenicity scoring sub-network.
54. The variant pathogenicity prediction network of any of clauses 1-53, wherein the variant encoding sub-network, the protein contact map generation sub-network, and the pathogenicity scoring sub-network are arranged as a single neural network.
55. The variant pathogenicity prediction network of any of clauses 1-54, wherein multiple trained instances of the single neural network are used as an ensemble for variant pathogenicity prediction during inference.
56. The variant pathogenicity prediction network of any of clauses 1-55, wherein the pathogenicity scoring sub-network is a fully connected network.
57. The variant pathogenicity prediction network of any of clauses 1-56, wherein the pathogenicity scoring sub-network comprises a pathogenicity indication generation layer (e.g., sigmoid, softmax) that generates the pathogenicity indication.
58. A computer-implemented method of variant pathogenicity prediction, including:
storing a reference amino acid sequence of a protein, and an alternative amino acid sequence of the protein that contains a variant amino acid caused by a variant nucleotide;
processing the alternative amino acid sequence, and generating a processed representation of the alternative amino acid sequence;
processing the reference amino acid sequence and the processed representation of the alternative amino acid sequence, and generating a protein contact map of the protein; and
processing the protein contact map, and generating a pathogenicity indication of the variant amino acid.
59. The computer-implemented of clause 58, further including storing an amino acid-wise primate conservation profile of the protein, and
wherein the processed representation of the alternative amino acid sequence is generated in response to processing the alternative amino acid sequence and the amino acid-wise primate conservation profile.
60. The computer-implemented method of any of clauses 58-59, further including storing an amino acid-wise mammal conservation profile of the protein, and
wherein the processed representation of the alternative amino acid sequence is generated in response to processing the alternative amino acid sequence and the amino acid-wise mammal conservation profile.
61. The computer-implemented method of any of clauses 58-60, further including storing an amino acid-wise vertebrate conservation profile of the protein, and
wherein the processed representation of the alternative amino acid sequence is generated in response to processing the alternative amino acid sequence and the amino acid-wise vertebrate conservation profile.
62. The computer-implemented method of any of clauses 58-61, wherein the processed representation of the alternative amino acid sequence is generated in response to processing the alternative amino acid sequence, the amino acid-wise primate conservation profile, the amino acid-wise mammal conservation profile, and the amino acid-wise vertebrate conservation profile.
63. The computer-implemented method of any of clauses 58-62, wherein the processed representation of the alternative amino acid sequence is generated in response to processing the alternative amino acid sequence, the amino acid-wise primate conservation profile, and the amino acid-wise mammal conservation profile.
64. The computer-implemented method of any of clauses 58-63, wherein the processed representation of the alternative amino acid sequence is generated in response to processing the alternative amino acid sequence, the amino acid-wise primate conservation profile, and the amino acid-wise vertebrate conservation profile.
65. The computer-implemented method of any of clauses 58-64, wherein the processed representation of the alternative amino acid sequence is generated in response to processing the alternative amino acid sequence, the amino acid-wise mammal conservation profile, and the amino acid-wise vertebrate conservation profile.
66. The computer-implemented method of any of clauses 58-65, further including storing an amino acid-wise secondary structure profile of the protein, and
wherein the protein contact map of the protein is generated in response to processing the reference amino acid sequence and the amino acid-wise secondary structure profile.
67. The computer-implemented method of any of clauses 58-66, further including storing an amino acid-wise solvent accessibility profile of the protein, and
wherein the protein contact map of the protein is generated in response to processing the reference amino acid sequence and the amino acid-wise solvent accessibility profile.
68. The computer-implemented method of any of clauses 58-67, further including storing an amino acid-wise position-specific frequency matrix of the protein, and
wherein the protein contact map of the protein is generated in response to processing the reference amino acid sequence and the amino acid-wise position-specific frequency matrix.
69. The computer-implemented method of any of clauses 58-68, further including storing an amino acid-wise position-specific scoring matrix of the protein, and
wherein the protein contact map of the protein is generated in response to processing the reference amino acid sequence and the amino acid-wise position-specific scoring matrix.
70. The computer-implemented method of any of clauses 58-69, wherein the protein contact map of the protein is generated in response to processing the reference amino acid sequence, the amino acid-wise secondary structure profile, the amino acid-wise solvent accessibility profile, the amino acid-wise position-specific frequency matrix, and the amino acid-wise position-specific scoring matrix.
71. The computer-implemented method of any of clauses 58-70, wherein the protein contact map of the protein is generated in response to processing the reference amino acid sequence, the amino acid-wise secondary structure profile, and the amino acid-wise solvent accessibility profile.
72. The computer-implemented method of any of clauses 58-71, wherein the protein contact map of the protein is generated in response to processing the reference amino acid sequence, the amino acid-wise secondary structure profile, and the amino acid-wise position-specific frequency matrix.
73. The computer-implemented method of any of clauses 58-72, wherein the protein contact map of the protein is generated in response to processing the reference amino acid sequence, the amino acid-wise secondary structure profile, and the amino acid-wise position-specific scoring matrix.
74. The computer-implemented method of any of clauses 58-73, wherein the protein contact map of the protein is generated in response to processing the reference amino acid sequence, the amino acid-wise solvent accessibility profile, and the amino acid-wise position-specific frequency matrix.
75. The computer-implemented method of any of clauses 58-74, wherein the protein contact map of the protein is generated in response to processing the reference amino acid sequence, the amino acid-wise solvent accessibility profile, and the amino acid-wise position-specific scoring matrix.
76. The computer-implemented method of any of clauses 58-75, wherein the protein contact map of the protein is generated in response to processing the reference amino acid sequence, the amino acid-wise position-specific frequency matrix, and the amino acid-wise position-specific scoring matrix.
77. The computer-implemented method of any of clauses 58-76, wherein the protein contact map of the protein is generated in response to processing the reference amino acid sequence, the amino acid-wise secondary structure profile, the amino acid-wise solvent accessibility profile, and the amino acid-wise position-specific frequency matrix.
78. The computer-implemented method of any of clauses 58-77, wherein the protein contact map of the protein is generated in response to processing the reference amino acid sequence, the amino acid-wise secondary structure profile, the amino acid-wise solvent accessibility profile, and the amino acid-wise position-specific scoring matrix.
79. A non-transitory computer readable storage medium impressed with computer program instructions to predict pathogenicity of variants, the instructions, when executed on a processor, implement a method comprising:
storing a reference amino acid sequence of a protein, and an alternative amino acid sequence of the protein that contains a variant amino acid caused by a variant nucleotide;
processing the alternative amino acid sequence, and generating a processed representation of the alternative amino acid sequence;
processing the reference amino acid sequence and the processed representation of the alternative amino acid sequence, and generating a protein contact map of the protein; and
processing the protein contact map, and generating a pathogenicity indication of the variant amino acid.
80. The non-transitory computer readable storage medium of clause 79, implementing the method further comprising storing an amino acid-wise primate conservation profile of the protein, and
wherein the processed representation of the alternative amino acid sequence is generated in response to processing the alternative amino acid sequence and the amino acid-wise primate conservation profile.
81. The non-transitory computer readable storage medium of any of clauses 79-80, implementing the method further comprising storing an amino acid-wise mammal conservation profile of the protein, and
wherein the processed representation of the alternative amino acid sequence is generated in response to processing the alternative amino acid sequence and the amino acid-wise mammal conservation profile.
82. The non-transitory computer readable storage medium of any of clauses 79-81, implementing the method further comprising storing an amino acid-wise vertebrate conservation profile of the protein, and
wherein the processed representation of the alternative amino acid sequence is generated in response to processing the alternative amino acid sequence and the amino acid-wise vertebrate conservation profile.
83. The non-transitory computer readable storage medium of any of clauses 79-82, wherein the processed representation of the alternative amino acid sequence is generated in response to processing the alternative amino acid sequence, the amino acid-wise primate conservation profile, the amino acid-wise mammal conservation profile, and the amino acid-wise vertebrate conservation profile.
84. The non-transitory computer readable storage medium of any of clauses 79-83, wherein the processed representation of the alternative amino acid sequence is generated in response to processing the alternative amino acid sequence, the amino acid-wise primate conservation profile, and the amino acid-wise mammal conservation profile.
85. The non-transitory computer readable storage medium of any of clauses 79-84, wherein the processed representation of the alternative amino acid sequence is generated in response to processing the alternative amino acid sequence, the amino acid-wise primate conservation profile, and the amino acid-wise vertebrate conservation profile.
86. The non-transitory computer readable storage medium of any of clauses 79-85, wherein the processed representation of the alternative amino acid sequence is generated in response to processing the alternative amino acid sequence, the amino acid-wise mammal conservation profile, and the amino acid-wise vertebrate conservation profile.
87. The non-transitory computer readable storage medium of any of clauses 79-86, implementing the method further comprising storing an amino acid-wise secondary structure profile of the protein, and
wherein the protein contact map of the protein is generated in response to processing the reference amino acid sequence and the amino acid-wise secondary structure profile.
88. The non-transitory computer readable storage medium of any of clauses 79-87, implementing the method further comprising storing an amino acid-wise solvent accessibility profile of the protein, and
wherein the protein contact map of the protein is generated in response to processing the reference amino acid sequence and the amino acid-wise solvent accessibility profile.
89. The non-transitory computer readable storage medium of any of clauses 79-88, implementing the method further comprising storing an amino acid-wise position-specific frequency matrix of the protein, and
wherein the protein contact map of the protein is generated in response to processing the reference amino acid sequence and the amino acid-wise position-specific frequency matrix.
90. The non-transitory computer readable storage medium of any of clauses 79-89, implementing the method further comprising storing an amino acid-wise position-specific scoring matrix of the protein, and
wherein the protein contact map of the protein is generated in response to processing the reference amino acid sequence and the amino acid-wise position-specific scoring matrix.
91. The non-transitory computer readable storage medium of any of clauses 79-90, wherein the protein contact map of the protein is generated in response to processing the reference amino acid sequence, the amino acid-wise secondary structure profile, the amino acid-wise solvent accessibility profile, the amino acid-wise position-specific frequency matrix, and the amino acid-wise position-specific scoring matrix.
92. The non-transitory computer readable storage medium of any of clauses 79-91, wherein the protein contact map of the protein is generated in response to processing the reference amino acid sequence, the amino acid-wise secondary structure profile, and the amino acid-wise solvent accessibility profile.
93. The non-transitory computer readable storage medium of any of clauses 79-92, wherein the protein contact map of the protein is generated in response to processing the reference amino acid sequence, the amino acid-wise secondary structure profile, and the amino acid-wise position-specific frequency matrix.
94. The non-transitory computer readable storage medium of any of clauses 79-93, wherein the protein contact map of the protein is generated in response to processing the reference amino acid sequence, the amino acid-wise secondary structure profile, and the amino acid-wise position-specific scoring matrix.
95. The non-transitory computer readable storage medium of any of clauses 79-94, wherein the protein contact map of the protein is generated in response to processing the reference amino acid sequence, the amino acid-wise solvent accessibility profile, and the amino acid-wise position-specific frequency matrix.
96. The non-transitory computer readable storage medium of any of clauses 79-95, wherein the protein contact map of the protein is generated in response to processing the reference amino acid sequence, the amino acid-wise solvent accessibility profile, and the amino acid-wise position-specific scoring matrix.
97. The non-transitory computer readable storage medium of any of clauses 79-96, wherein the protein contact map of the protein is generated in response to processing the reference amino acid sequence, the amino acid-wise position-specific frequency matrix, and the amino acid-wise position-specific scoring matrix.
98. The non-transitory computer readable storage medium of any of clauses 79-97, wherein the protein contact map of the protein is generated in response to processing the reference amino acid sequence, the amino acid-wise secondary structure profile, the amino acid-wise solvent accessibility profile, and the amino acid-wise position-specific frequency matrix.
99. The non-transitory computer readable storage medium of any of clauses 79-98, wherein the protein contact map of the protein is generated in response to processing the reference amino acid sequence, the amino acid-wise secondary structure profile, the amino acid-wise solvent accessibility profile, and the amino acid-wise position-specific scoring matrix.
100. A system, comprising:
a variant pathogenicity determiner configured to determine pathogenicity of variants that cause amino acid variants in proteins based on processing protein contact maps of the proteins.
101. A computer-implemented method, including:
determining pathogenicity of variants that cause amino acid variants in proteins based on processing protein contact maps of the proteins.
102. A non-transitory computer readable storage medium impressed with computer program instructions to predict pathogenicity of variants, the instructions, when executed on a processor, implement a method comprising: determining pathogenicity of variants that cause amino acid variants in proteins based on processing protein contact maps of the proteins.

### Clauses Set 2

1. A variant pathogenicity classifier, comprising:
memory storing (i) a reference amino acid sequence of a protein, (ii) an alternative amino acid sequence of the protein that contains a variant amino acid caused by a variant nucleotide, and (iii) a protein contact map of the protein; and
runtime logic, having access to the memory, configured to provide (i) the reference amino acid sequence, (ii) the alternative amino acid sequence, and (iii) the protein contact map as input to a first neural network, and to cause the first neural network to generate a pathogenicity indication of the variant amino acid as output in response to processing (i) the reference amino acid sequence, (ii) the alternative amino acid sequence, and (iii) the protein contact map.
2. The variant pathogenicity classifier of clause 1, wherein the memory stores an amino acid-wise primate conservation profile of the protein, an amino acid-wise mammal conservation profile of the protein, and an amino acid-wise vertebrate conservation profile of the protein, and
wherein the runtime logic further configured to provide (i) the reference amino acid sequence, (ii) the alternative amino acid sequence, (iii) the protein contact map, (iv) the amino acid-wise primate conservation profile, (v) the amino acid-wise mammal conservation profile, and (vi) the amino acid-wise vertebrate conservation profile as input to the first neural network, and to cause the first neural network to generate the pathogenicity indication of the variant amino acid as output in response to processing (i) the reference amino acid sequence, (ii) the alternative amino acid sequence, (iii) the protein contact map, (iv) the amino acid-wise primate conservation profile, (v) the amino acid-wise mammal conservation profile, and (vi) the amino acid-wise vertebrate conservation profile.
3. The variant pathogenicity classifier of any of clauses 1-2, wherein the reference amino acid sequence has L amino acids, wherein the alternative amino acid sequence has L amino acids.
4. The variant pathogenicity classifier of any of clauses 1-3, wherein the reference amino acid sequence is characterized as a reference one-hot encoded matrix of size L by C, where C denotes twenty amino acid categories, wherein the alternative amino acid sequence is characterized as an alternative one-hot encoded matrix of size L by C.
5. The variant pathogenicity classifier of any of clauses 1-4, wherein the amino acid-wise primate conservation profile is of size L by C, wherein the amino acid-wise mammal conservation profile is of size L by C, and wherein the amino acid-wise vertebrate conservation profile is of size L by C.
6. The variant pathogenicity classifier of any of clauses 1-5, wherein the first neural network is a first convolutional neural network.
7. The variant pathogenicity classifier of any of clauses 1-6, wherein the first convolutional neural network comprises (i) one or more one-dimensional (1D) convolution layers, followed by (ii) a first set of residual blocks with 1D convolutions, followed by (iii) a second set of residual blocks with 1D convolutions, followed by (iv) a spatial dimensionality augmentation layer, followed by (v) a first set of residual blocks with two-dimensional (2D) convolutions, followed by (vi) one or more 2D convolution layers, followed by (vii) one or more fully connected layers, and followed by (viii) a pathogenicity indication generation layer.
8. The variant pathogenicity classifier of any of clauses 1-7, wherein a spatial dimensionality (e.g., width×height) of an input processed by a first 1D convolution layer in the one or more 1D convolution layers is L by 1.
9. The variant pathogenicity classifier of any of clauses 1-8, wherein a depth dimensionality of the input processed by the first 1D convolution is D (e.g., 100), where D=C+C+C+C+C.
10. The variant pathogenicity classifier of any of clauses 1-9, wherein the first set of residual blocks with 1D convolutions has N1 residual blocks (e.g., N1=2, 3, 4, 5), the second set of residual blocks with 1D convolutions has N2 residual blocks (e.g., N2=2, 3, 4, 5), and the first set of residual blocks with 2D convolutions has N3 residual blocks (e.g., N3=2, 3, 4, 5).
11. The variant pathogenicity classifier of any of clauses 1-10, wherein an output of a final residual block in the second set of residual blocks with 1D convolutions is processed by the spatial dimensionality augmentation layer to generate a spatially augmented output.
12. The variant pathogenicity classifier of any of clauses 1-11, wherein the spatial dimensionality augmentation layer is configured to apply an outer product on the output of the final residual block to generate the spatially augmented output.
13. The variant pathogenicity classifier of any of clauses 1-12, wherein a spatial dimensionality of the spatially augmented output is L by L.
14. The variant pathogenicity classifier of any of clauses 1-13, wherein the spatially augmented output is combined (e.g., concatenated, summed) with the protein contact map to generate an intermediate combined output.
15. The variant pathogenicity classifier of any of clauses 1-14, wherein the intermediate combined output is processed by a first residual block in the first set of residual blocks with 2D convolutions.
16. The variant pathogenicity classifier of any of clauses 1-15, wherein the protein contact map is provided as input to a first layer of the first neural network.
17. The variant pathogenicity classifier of any of clauses 1-16, wherein the protein contact map is provided as input to one or more intermediate layers of the first neural network.
18. The variant pathogenicity classifier of any of clauses 1-17, wherein the protein contact map is provided as input to a final layer of the first neural network.
19. The variant pathogenicity classifier of any of clauses 1-18, wherein the protein contact map is combined (e.g., concatenated, summed) with an input to the first neural network.
20. The variant pathogenicity classifier of any of clauses 1-19, wherein the protein contact map is combined (e.g., concatenated, summed) with one or more intermediate outputs of the first neural network.
21. The variant pathogenicity classifier of any of clauses 1-20, wherein the protein contact map is combined (e.g., concatenated, summed) with a final output of the first neural network.
22. The variant pathogenicity classifier of any of clauses 1-21, wherein the protein contact map is generated by a second neural network in response to processing (i) the reference amino acid sequence and at least one of (ii) the amino acid-wise protein secondary structure profile, (iii) the amino acid-wise solvent accessibility profile, (iv) the amino acid-wise position-specific scoring matrix, and (v) the amino acid-wise position-specific frequency matrix.
23. The variant pathogenicity classifier of any of clauses 1-22, wherein the protein contact map has a total dimensionality of L by L by K (e.g., K=10, 15, 20, 25).
24. The variant pathogenicity classifier of any of clauses 1-23, wherein the second neural network is a second convolutional neural network.
25. The variant pathogenicity classifier of any of clauses 1-24, wherein the second convolutional neural network comprises (i) one or more 1D convolution layers, followed by (ii) one or more residual blocks with 1D convolutions, followed by (iii) a spatial dimensionality augmentation layer, followed by (iv) one or more residual blocks with 2D convolutions, and followed by (v) one or more 2D convolution layers.
26. The variant pathogenicity classifier of any of clauses 1-25, wherein the first convolutional neural network uses convolution filters of different filter sizes (e.g., 5×2, 2×5).
27. The variant pathogenicity classifier of any of clauses 1-26, wherein the first convolutional neural network does not include the one or more fully connected layers.
28. The variant pathogenicity classifier of any of clauses 1-27, wherein multiple trained instances of the first neural network are used as an ensemble for variant pathogenicity prediction during inference.
29. The variant pathogenicity classifier of any of clauses 1-28, wherein the first and second sets of residual blocks with 1D convolutions execute a series of 1D convolutional transformations of 1D sequential features in (i) the reference amino acid sequence, (ii) the alternative amino acid sequence, and at least one of (iii) the amino acid-wise primate conservation profile, (iv) the amino acid-wise mammal conservation profile, and (v) the amino acid-wise vertebrate conservation profile.
30. The variant pathogenicity classifier of any of clauses 1-29, wherein the first set of residual blocks with 2D convolutions execute a series of 2D convolutional transformations of 2D spatial features in (i) the protein contact map and (ii) the intermediate combined output.
31. The variant pathogenicity classifier of any of clauses 1-30, wherein the first set of residual blocks with 2D convolutions extract spatial interactions from the protein contact map about pathogenicity association between those amino acids of the protein that are more proximate in the three-dimensional (3D) structure of the protein than in the reference and alternative amino acid sequences.
32. A computer-implemented method of variant pathogenicity classification, including:
storing (i) a reference amino acid sequence of a protein, (ii) an alternative amino acid sequence of the protein that contains a variant amino acid caused by a variant nucleotide, and (iii) a protein contact map of the protein; and
providing (i) the reference amino acid sequence, (ii) the alternative amino acid sequence, and (iii) the protein contact map as input to a first neural network, and causing the first neural network to generate a pathogenicity indication of the variant amino acid as output in response to processing (i) the reference amino acid sequence, (ii) the alternative amino acid sequence, and (iii) the protein contact map.
33. The computer-implemented method of clause 32, further including storing an amino acid-wise primate conservation profile of the protein, an amino acid-wise mammal conservation profile of the protein, and an amino acid-wise vertebrate conservation profile of the protein, and
providing (i) the reference amino acid sequence, (ii) the alternative amino acid sequence, (iii) the protein contact map, (iv) the amino acid-wise primate conservation profile, (v) the amino acid-wise mammal conservation profile, and (vi) the amino acid-wise vertebrate conservation profile as input to the first neural network, and causing the first neural network to generate the pathogenicity indication of the variant amino acid as output in response to processing (i) the reference amino acid sequence, (ii) the alternative amino acid sequence, (iii) the protein contact map, (iv) the amino acid-wise primate conservation profile, (v) the amino acid-wise mammal conservation profile, and (vi) the amino acid-wise vertebrate conservation profile.
34. The computer-implemented method of any of clauses 32-33, wherein the reference amino acid sequence has L amino acids, wherein the alternative amino acid sequence has L amino acids.
35. The computer-implemented method of any of clauses 32-34, wherein the reference amino acid sequence is characterized as a reference one-hot encoded matrix of size L by C, where C denotes twenty amino acid categories, wherein the alternative amino acid sequence is characterized as an alternative one-hot encoded matrix of size L by C.
36. The computer-implemented method of any of clauses 32-35, wherein the amino acid-wise primate conservation profile is of size L by C, wherein the amino acid-wise mammal conservation profile is of size L by C, and wherein the amino acid-wise vertebrate conservation profile is of size L by C.
37. The computer-implemented method of any of clauses 32-36, wherein the first neural network is a first convolutional neural network.
38. The computer-implemented method of any of clauses 32-37, wherein the first convolutional neural network comprises (i) one or more one-dimensional (1D) convolution layers, followed by (ii) a first set of residual blocks with 1D convolutions, followed by (iii) a second set of residual blocks with 1D convolutions, followed by (iv) a spatial dimensionality augmentation layer, followed by (v) a first set of residual blocks with two-dimensional (2D) convolutions, followed by (vi) one or more 2D convolution layers, followed by (vii) one or more fully connected layers, and followed by (viii) a pathogenicity indication generation layer.
39. The computer-implemented method of any of clauses 32-38, wherein a spatial dimensionality (e.g., width×height) of an input processed by a first 1D convolution layer in the one or more 1D convolution layers is L by 1.
40. The computer-implemented method of any of clauses 32-39, wherein a depth dimensionality of the input processed by the first 1D convolution is D (e.g., 100), where D=C+C+C+C+C.
41. The computer-implemented method of any of clauses 32-40, wherein the first set of residual blocks with 1D convolutions has N1 residual blocks (e.g., N1=2, 3, 4, 5), the second set of residual blocks with 1D convolutions has N2 residual blocks (e.g., N2=2, 3, 4, 5), and the first set of residual blocks with 2D convolutions has N3 residual blocks (e.g., N3=2, 3, 4, 5).
42. The computer-implemented method of any of clauses 32-41, wherein an output of a final residual block in the second set of residual blocks with 1D convolutions is processed by the spatial dimensionality augmentation layer to generate a spatially augmented output.
43. The computer-implemented method of any of clauses 32-42, wherein the spatial dimensionality augmentation layer is configured to apply an outer product on the output of the final residual block to generate the spatially augmented output.
44. The computer-implemented method of any of clauses 32-43, wherein a spatial dimensionality of the spatially augmented output is L by L.
45. The computer-implemented method of any of clauses 32-44, wherein the spatially augmented output is combined (e.g., concatenated, summed) with the protein contact map to generate an intermediate combined output.
46. The computer-implemented method of any of clauses 32-45, wherein the intermediate combined output is processed by a first residual block in the first set of residual blocks with 2D convolutions.
47. The computer-implemented method of any of clauses 32-46, wherein the protein contact map is provided as input to a first layer of the first neural network.
48. The computer-implemented method of any of clauses 32-47, wherein the protein contact map is provided as input to one or more intermediate layers of the first neural network.
49. The computer-implemented method of any of clauses 32-48, wherein the protein contact map is provided as input to a final layer of the first neural network.
50. The computer-implemented method of any of clauses 32-49, wherein the protein contact map is combined (e.g., concatenated, summed) with an input to the first neural network.
51. The computer-implemented method of any of clauses 32-50, wherein the protein contact map is combined (e.g., concatenated, summed) with one or more intermediate outputs of the first neural network.
52. The computer-implemented method of any of clauses 32-51, wherein the protein contact map is combined (e.g., concatenated, summed) with a final output of the first neural network.
53. The computer-implemented method of any of clauses 32-52, wherein the protein contact map is generated by a second neural network in response to processing (i) the reference amino acid sequence and at least one of (ii) the amino acid-wise protein secondary structure profile, (iii) the amino acid-wise solvent accessibility profile, (iv) the amino acid-wise position-specific scoring matrix, and (v) the amino acid-wise position-specific frequency matrix.
54. The computer-implemented method of any of clauses 32-53, wherein the protein contact map has a total dimensionality of L by L by K (e.g., K=10, 15, 20, 25).
55. The computer-implemented method of any of clauses 32-54, wherein the second neural network is a second convolutional neural network.
56. The computer-implemented method of any of clauses 32-55, wherein the second convolutional neural network comprises (i) one or more 1D convolution layers, followed by (ii) one or more residual blocks with 1D convolutions, followed by (iii) a spatial dimensionality augmentation layer, followed by (iv) one or more residual blocks with 2D convolutions, and followed by (v) one or more 2D convolution layers.
57. The computer-implemented method of any of clauses 32-56, wherein the first convolutional neural network uses convolution filters of different filter sizes (e.g., 5×2, 2×5).
58. The computer-implemented method of any of clauses 32-57, wherein the first convolutional neural network does not include the one or more fully connected layers.
59. The computer-implemented method of any of clauses 32-58, wherein multiple trained instances of the first neural network are used as an ensemble for variant pathogenicity prediction during inference.
60. The computer-implemented method of any of clauses 32-59, wherein the first and second sets of residual blocks with 1D convolutions execute a series of 1D convolutional transformations of 1D sequential features in (i) the reference amino acid sequence, (ii) the alternative amino acid sequence, and at least one of (iii) the amino acid-wise primate conservation profile, (iv) the amino acid-wise mammal conservation profile, and (v) the amino acid-wise vertebrate conservation profile.
61. The computer-implemented method of any of clauses 32-60, wherein the first set of residual blocks with 2D convolutions execute a series of 2D convolutional transformations of 2D spatial features in (i) the protein contact map and (ii) the intermediate combined output.
62. The computer-implemented method of any of clauses 32-61, wherein the first set of residual blocks with 2D convolutions extract spatial interactions from the protein contact map about pathogenicity association between those amino acids of the protein that are more proximate in the three-dimensional (3D) structure of the protein than in the reference and alternative amino acid sequences.
63. A non-transitory computer readable storage medium impressed with computer program instructions to classify pathogenicity of variants, the instructions, when executed on a processor, implement a method comprising:
storing (i) a reference amino acid sequence of a protein, (ii) an alternative amino acid sequence of the protein that contains a variant amino acid caused by a variant nucleotide, and (iii) a protein contact map of the protein; and
providing (i) the reference amino acid sequence, (ii) the alternative amino acid sequence, and (iii) the protein contact map as input to a first neural network, and causing the first neural network to generate a pathogenicity indication of the variant amino acid as output in response to processing (i) the reference amino acid sequence, (ii) the alternative amino acid sequence, and (iii) the protein contact map.
64. The non-transitory computer readable storage medium of clause 63, implementing the method further comprising storing an amino acid-wise primate conservation profile of the protein, an amino acid-wise mammal conservation profile of the protein, and an amino acid-wise vertebrate conservation profile of the protein, and
providing (i) the reference amino acid sequence, (ii) the alternative amino acid sequence, (iii) the protein contact map, (iv) the amino acid-wise primate conservation profile, (v) the amino acid-wise mammal conservation profile, and (vi) the amino acid-wise vertebrate conservation profile as input to the first neural network, and causing the first neural network to generate the pathogenicity indication of the variant amino acid as output in response to processing (i) the reference amino acid sequence, (ii) the alternative amino acid sequence, (iii) the protein contact map, (iv) the amino acid-wise primate conservation profile, (v) the amino acid-wise mammal conservation profile, and (vi) the amino acid-wise vertebrate conservation profile.
65. The non-transitory computer readable storage medium of any of clauses 63-64, wherein the reference amino acid sequence has L amino acids, wherein the alternative amino acid sequence has L amino acids.
66. The non-transitory computer readable storage medium of any of clauses 63-65, wherein the reference amino acid sequence is characterized as a reference one-hot encoded matrix of size L by C, where C denotes twenty amino acid categories, wherein the alternative amino acid sequence is characterized as an alternative one-hot encoded matrix of size L by C.
67. The non-transitory computer readable storage medium of any of clauses 63-66, wherein the amino acid-wise primate conservation profile is of size L by C, wherein the amino acid-wise mammal conservation profile is of size L by C, and wherein the amino acid-wise vertebrate conservation profile is of size L by C.
68. The non-transitory computer readable storage medium of any of clauses 63-67, wherein the first neural network is a first convolutional neural network.
69. The non-transitory computer readable storage medium of any of clauses 63-68, wherein the first convolutional neural network comprises (i) one or more one-dimensional (1D) convolution layers, followed by (ii) a first set of residual blocks with 1D convolutions, followed by (iii) a second set of residual blocks with 1D convolutions, followed by (iv) a spatial dimensionality augmentation layer, followed by (v) a first set of residual blocks with two-dimensional (2D) convolutions, followed by (vi) one or more 2D convolution layers, followed by (vii) one or more fully connected layers, and followed by (viii) a pathogenicity indication generation layer.
70. The non-transitory computer readable storage medium of any of clauses 63-69, wherein a spatial dimensionality (e.g., width×height) of an input processed by a first 1D convolution layer in the one or more 1D convolution layers is L by 1.
71. The non-transitory computer readable storage medium of any of clauses 63-70, wherein a depth dimensionality of the input processed by the first 1D convolution is D (e.g., 100), where D=C+C+C+C+C.
72. The non-transitory computer readable storage medium of any of clauses 63-71, wherein the first set of residual blocks with 1D convolutions has N1 residual blocks (e.g., N1=2, 3, 4, 5), the second set of residual blocks with 1D convolutions has N2 residual blocks (e.g., N2=2, 3, 4, 5), and the first set of residual blocks with 2D convolutions has N3 residual blocks (e.g., N3=2, 3, 4, 5).
73. The non-transitory computer readable storage medium of any of clauses 63-72, wherein an output of a final residual block in the second set of residual blocks with 1D convolutions is processed by the spatial dimensionality augmentation layer to generate a spatially augmented output.
74. The non-transitory computer readable storage medium of any of clauses 63-73, wherein the spatial dimensionality augmentation layer is configured to apply an outer product on the output of the final residual block to generate the spatially augmented output.
75. The non-transitory computer readable storage medium of any of clauses 63-74, wherein a spatial dimensionality of the spatially augmented output is L by L.
76. The non-transitory computer readable storage medium of any of clauses 63-75, wherein the spatially augmented output is combined (e.g., concatenated, summed) with the protein contact map to generate an intermediate combined output.
77. The non-transitory computer readable storage medium of any of clauses 63-76, wherein the intermediate combined output is processed by a first residual block in the first set of residual blocks with 2D convolutions.
78. The non-transitory computer readable storage medium of any of clauses 63-77, wherein the protein contact map is provided as input to a first layer of the first neural network.
79. The non-transitory computer readable storage medium of any of clauses 63-78, wherein the protein contact map is provided as input to one or more intermediate layers of the first neural network.
80. The non-transitory computer readable storage medium of any of clauses 63-79, wherein the protein contact map is provided as input to a final layer of the first neural network.
81. The non-transitory computer readable storage medium of any of clauses 63-80, wherein the protein contact map is combined (e.g., concatenated, summed) with an input to the first neural network.
82. The non-transitory computer readable storage medium of any of clauses 63-81, wherein the protein contact map is combined (e.g., concatenated, summed) with one or more intermediate outputs of the first neural network.
83. The non-transitory computer readable storage medium of any of clauses 63-82, wherein the protein contact map is combined (e.g., concatenated, summed) with a final output of the first neural network.
84. The non-transitory computer readable storage medium of any of clauses 63-83, wherein the protein contact map is generated by a second neural network in response to processing (i) the reference amino acid sequence and at least one of (ii) the amino acid-wise protein secondary structure profile, (iii) the amino acid-wise solvent accessibility profile, (iv) the amino acid-wise position-specific scoring matrix, and (v) the amino acid-wise position-specific frequency matrix.
85. The non-transitory computer readable storage medium of any of clauses 63-84, wherein the protein contact map has a total dimensionality of L by L by K (e.g., K=10, 15, 20, 25).
86. The non-transitory computer readable storage medium of any of clauses 63-85, wherein the second neural network is a second convolutional neural network.
87. The non-transitory computer readable storage medium of any of clauses 63-86, wherein the second convolutional neural network comprises (i) one or more 1D convolution layers, followed by (ii) one or more residual blocks with 1D convolutions, followed by (iii) a spatial dimensionality augmentation layer, followed by (iv) one or more residual blocks with 2D convolutions, and followed by (v) one or more 2D convolution layers.
88. The non-transitory computer readable storage medium of any of clauses 63-87, wherein the first convolutional neural network uses convolution filters of different filter sizes (e.g., 5×2, 2×5).
89. The non-transitory computer readable storage medium of any of clauses 63-88, wherein the first convolutional neural network does not include the one or more fully connected layers.
90. The non-transitory computer readable storage medium of any of clauses 63-89, wherein multiple trained instances of the first neural network are used as an ensemble for variant pathogenicity prediction during inference.
91. The non-transitory computer readable storage medium of any of clauses 63-90, wherein the first and second sets of residual blocks with 1D convolutions execute a series of 1D convolutional transformations of 1D sequential features in (i) the reference amino acid sequence, (ii) the alternative amino acid sequence, and at least one of (iii) the amino acid-wise primate conservation profile, (iv) the amino acid-wise mammal conservation profile, and (v) the amino acid-wise vertebrate conservation profile.
92. The non-transitory computer readable storage medium of any of clauses 63-91, wherein the first set of residual blocks with 2D convolutions execute a series of 2D convolutional transformations of 2D spatial features in (i) the protein contact map and (ii) the intermediate combined output.
93. The non-transitory computer readable storage medium of any of clauses 63-92, wherein the first set of residual blocks with 2D convolutions extract spatial interactions from the protein contact map about pathogenicity association between those amino acids of the protein that are more proximate in the three-dimensional (3D) structure of the protein than in the reference and alternative amino acid sequences.

While the present invention is disclosed by reference to the preferred implementations and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims

What is claimed is:

1. A variant pathogenicity prediction network, comprising:
memory storing a reference amino acid sequence of a protein, and an alternative amino acid sequence of the protein that contains a variant amino acid caused by a variant nucleotide;
a variant encoding sub-network, having access to the memory, configured to process the alternative amino acid sequence, and generate a processed representation of the alternative amino acid sequence;
a protein contact map generation sub-network, in communication with the variant encoding sub-network, configured to process the reference amino acid sequence and the processed representation of the alternative amino acid sequence, and generate a protein contact map of the protein; and
a pathogenicity scoring sub-network, in communication with the protein contact map generation sub-network, configured to process the protein contact map, and generate a pathogenicity indication of the variant amino acid.

2. The variant pathogenicity prediction network of claim 1, wherein the memory further stores an amino acid-wise primate conservation profile of the protein, and
wherein the processed representation of the alternative amino acid sequence is generated by the variant encoding sub-network in response to processing the alternative amino acid sequence and the amino acid-wise primate conservation profile.

3. The variant pathogenicity prediction network of claim 2, wherein the memory further stores an amino acid-wise mammal conservation profile of the protein, and
wherein the processed representation of the alternative amino acid sequence is generated by the variant encoding sub-network in response to processing the alternative amino acid sequence and the amino acid-wise mammal conservation profile.

4. The variant pathogenicity prediction network of claim 3, wherein the memory further stores an amino acid-wise vertebrate conservation profile of the protein, and
wherein the processed representation of the alternative amino acid sequence is generated by the variant encoding sub-network in response to processing the alternative amino acid sequence and the amino acid-wise vertebrate conservation profile.

5. The variant pathogenicity prediction network of claim 4, wherein the processed representation of the alternative amino acid sequence is generated by the variant encoding sub-network in response to processing the alternative amino acid sequence, the amino acid-wise primate conservation profile, the amino acid-wise mammal conservation profile, and the amino acid-wise vertebrate conservation profile.

6. The variant pathogenicity prediction network of claim 4, wherein the processed representation of the alternative amino acid sequence is generated by the variant encoding sub-network in response to processing the alternative amino acid sequence, the amino acid-wise primate conservation profile, and the amino acid-wise mammal conservation profile.

7. The variant pathogenicity prediction network of claim 4, wherein the processed representation of the alternative amino acid sequence is generated by the variant encoding sub-network in response to processing the alternative amino acid sequence, the amino acid-wise primate conservation profile, and the amino acid-wise vertebrate conservation profile.

8. The variant pathogenicity prediction network of claim 4, wherein the processed representation of the alternative amino acid sequence is generated by the variant encoding sub-network in response to processing the alternative amino acid sequence, the amino acid-wise mammal conservation profile, and the amino acid-wise vertebrate conservation profile.

9. The variant pathogenicity prediction network of claim 1, wherein the memory further stores an amino acid-wise secondary structure profile of the protein, and
wherein the protein contact map of the protein is generated by the protein contact map generation sub-network in response to processing the reference amino acid sequence and the amino acid-wise secondary structure profile.

10. The variant pathogenicity prediction network of claim 9, wherein the memory further stores an amino acid-wise solvent accessibility profile of the protein, and
wherein the protein contact map of the protein is generated by the protein contact map generation sub-network in response to processing the reference amino acid sequence and the amino acid-wise solvent accessibility profile.

11. The variant pathogenicity prediction network of claim 10, wherein the protein contact map of the protein is generated by the protein contact map generation sub-network in response to processing the reference amino acid sequence, the amino acid-wise secondary structure profile, and the amino acid-wise solvent accessibility profile.

12. The variant pathogenicity prediction network of claim 10, wherein the memory further stores an amino acid-wise position-specific frequency matrix of the protein, and
wherein the protein contact map of the protein is generated by the protein contact map generation sub-network in response to processing the reference amino acid sequence and the amino acid-wise position-specific frequency matrix.

13. The variant pathogenicity prediction network of claim 12, wherein the protein contact map of the protein is generated by the protein contact map generation sub-network in response to processing the reference amino acid sequence, the amino acid-wise secondary structure profile, and the amino acid-wise position-specific frequency matrix.

14. The variant pathogenicity prediction network of claim 12, wherein the protein contact map of the protein is generated by the protein contact map generation sub-network in response to processing the reference amino acid sequence, the amino acid-wise solvent accessibility profile, and the amino acid-wise position-specific frequency matrix.

15. The variant pathogenicity prediction network of claim 12, wherein the protein contact map of the protein is generated by the protein contact map generation sub-network in response to processing the reference amino acid sequence, the amino acid-wise secondary structure profile, the amino acid-wise solvent accessibility profile, and the amino acid-wise position-specific frequency matrix.

16. The variant pathogenicity prediction network of claim 12, wherein the memory further stores an amino acid-wise position-specific scoring matrix of the protein, and
wherein the protein contact map of the protein is generated by the protein contact map generation sub-network in response to processing the reference amino acid sequence and the amino acid-wise position-specific scoring matrix.

17. The variant pathogenicity prediction network of claim 12, wherein the protein contact map of the protein is generated by the protein contact map generation sub-network in response to processing the reference amino acid sequence, the amino acid-wise secondary structure profile, the amino acid-wise solvent accessibility profile, the amino acid-wise position-specific frequency matrix, and the amino acid-wise position-specific scoring matrix.

18. The variant pathogenicity prediction network of claim 12, wherein the protein contact map of the protein is generated by the protein contact map generation sub-network in response to processing the reference amino acid sequence, the amino acid-wise secondary structure profile, and the amino acid-wise position-specific scoring matrix.

19. The variant pathogenicity prediction network of claim 12, wherein the protein contact map of the protein is generated by the protein contact map generation sub-network in response to processing the reference amino acid sequence, the amino acid-wise solvent accessibility profile, and the amino acid-wise position-specific scoring matrix.

20. The variant pathogenicity prediction network of claim 12, wherein the protein contact map of the protein is generated by the protein contact map generation sub-network in response to processing the reference amino acid sequence, the amino acid-wise position-specific frequency matrix, and the amino acid-wise position-specific scoring matrix.

21. The variant pathogenicity prediction network of claim 12, wherein the protein contact map of the protein is generated by the protein contact map generation sub-network in response to processing the reference amino acid sequence, the amino acid-wise secondary structure profile, the amino acid-wise solvent accessibility profile, and the amino acid-wise position-specific scoring matrix.

22. The variant pathogenicity prediction network of claim 1, wherein the processed representation of the alternative amino acid sequence is provided as input to a first layer of the protein contact map generation sub-network.

23. The variant pathogenicity prediction network of claim 22, wherein the processed representation of the alternative amino acid sequence is provided as input to one or more intermediate layers of the protein contact map generation sub-network.

24. The variant pathogenicity prediction network of claim 23, wherein the processed representation of the alternative amino acid sequence is provided as input to a final layer of the protein contact map generation sub-network.

25. A computer-implemented method of variant pathogenicity prediction, including:
storing a reference amino acid sequence of a protein, and an alternative amino acid sequence of the protein that contains a variant amino acid caused by a variant nucleotide;
processing the alternative amino acid sequence, and generating a processed representation of the alternative amino acid sequence;
processing the reference amino acid sequence and the processed representation of the alternative amino acid sequence, and generating a protein contact map of the protein; and
processing the protein contact map, and generating a pathogenicity indication of the variant amino acid.

26. The computer-implemented of claim 25, further including storing an amino acid-wise primate conservation profile of the protein, and
wherein the processed representation of the alternative amino acid sequence is generated in response to processing the alternative amino acid sequence and the amino acid-wise primate conservation profile.

27. The computer-implemented method of claim 26, further including storing an amino acid-wise mammal conservation profile of the protein, and
wherein the processed representation of the alternative amino acid sequence is generated in response to processing the alternative amino acid sequence and the amino acid-wise mammal conservation profile.

28. The computer-implemented method of claim 27, further including storing an amino acid-wise vertebrate conservation profile of the protein, and
wherein the processed representation of the alternative amino acid sequence is generated in response to processing the alternative amino acid sequence and the amino acid-wise vertebrate conservation profile.

29. The computer-implemented method of claim 28, wherein the processed representation of the alternative amino acid sequence is generated in response to processing the alternative amino acid sequence, the amino acid-wise primate conservation profile, the amino acid-wise mammal conservation profile, and the amino acid-wise vertebrate conservation profile.

30. A non-transitory computer readable storage medium impressed with computer program instructions to predict pathogenicity of variants, the instructions, when executed on a processor, implement a method comprising:
storing a reference amino acid sequence of a protein, and an alternative amino acid sequence of the protein that contains a variant amino acid caused by a variant nucleotide;
processing the alternative amino acid sequence, and generating a processed representation of the alternative amino acid sequence;
processing the reference amino acid sequence and the processed representation of the alternative amino acid sequence, and generating a protein contact map of the protein; and
processing the protein contact map, and generating a pathogenicity indication of the variant amino acid.
